(19) United States
(12) Patent Application Publication
(10) Pub. No.: US 2022/0146502 A1
(43) Pub. Date: May 12, 2022

(54) HIGH-SENSITIVITY ASSAY

(71) Applicant: SENTILUS HOLDCO, LLC, Norcross, GA (US)

(72) Inventors: Reiner BABIEL, Seehausen a. Staffelsee (DE); Tom FRAME, Norcross, GA (US); Sukanta BANERJEE, Warren, NJ (US); Tiruneh HAILEMARIAM, Secaucus, NJ (US); Hui HUANG, Warren, NJ (US); Efstathia MARINAKOS, Norcross, GA (US); Angus HUCKNALL, Durham, NC (US); Vinalia TJONG, Painted Post, NY (US)

(21) Appl. No.: 17/427,799

(22) PCT Filed: Jan. 31, 2020
(86) PCT No.: PCT/US20/16212

§ 371 Date: Aug. 2, 2021

Related U.S. Application Data
(60) Provisional application No. 62/799,779, filed on Feb. 1, 2019.

(51) Int. Cl. G01N 33/545; G01N 33/569; G01N 33/80; G01N 33/543

(52) U.S. Cl. CPC G01N 33/545; G01N 33/54366; G01N 33/80; G01N 33/56911

(57) ABSTRACT

Disclosed herein are biological assays, screening formats, detection devices, and related methods of use. More specifically, disclosed herein are assay formats, microarrays, devices, methods of making the same, and methods of screening, detecting a target analyte, and methods of diagnosing an individual with a disease or condition when a target analyte associated with the disease or condition is detected.

### BACKGROUND

Sensitive detection of antibodies to infectious disease agents and other human antigens in blood donors is crucial for a safe transfusion of collected blood and components. The screening of the absence for these antibodies must be highly specific as donors tested positive may be excluded from further donations.

Currently, there are assays in use that allow the detection of human antibodies, such as anti-Treponema pallidum (TP) or anti-CMV assays. However, full screening in a blood donor setting requires additional testing of antibodies, including HIV, HBV, HCV, and HTLV, etc., and such testing typically detects antibodies directed against one of the targets in each assay. In addition, these assays may detect only one subset of antibodies, i.e., IgG or IgM. A sensitive and lasting detection of Infectious Disease (ID) antibodies requires the detection of both human IgG and IgM. Unfortunately, non-specific reactivity is typically seen with anti-human IgG/IgM formats.

In addition, current methods of screening, e.g., in the ID area, suffer from several shortcomings including high cost, complexity of the assay, time required to perform the assay, and sensitivity of the assay. For example, enzyme-linked immunosorbent assay (ELISA) can be used to test for infectious disease markers. Multiplex ELISA can test an array of four infectious agents at a cost of about 14 USD and requires on average about 4.5 hours of technician time. However, these tests require the use of expensive laboratory equipment and a sizable volume of serum or plasma. Conventional testing in many instances also requires transport of the biological sample because, typically, blood is being taken from a patient at the point-of-care (POC) and is then transferred to a centralized laboratory for analyses.

Thus, there is a need to develop a highly specific detection format that allows the simultaneous and individual detection of multiple antibodies with a single incubation in one reaction with reduced background signal, and an assay that can significantly increase the efficiency and time to report a diagnosis to a donor/patient by avoiding reflexive, sequential testing procedures.

Further, during antibody detection for antigen A and antigen B with anti-human IgG and anti-human IgM in a surface-based assay, some plasmas cause a high background signal, making detection of the antigen difficult. The high background signal has been observed on POEGMA-based substrates, Immucor's in-house developed substrates that contain PEG, and commercial substrates including AMI Silane 1, AMI Silane 2, AMI Polymer 1, AMI polymer 2, AMI Polymer 3 (Applied Microarrays (Tempe, Ariz.)), NEXTERION® Aldehydesilane (AL), NEXTERION® epoxysilane, NEXTERION® 3-D Hydrogel (Schott, Ky., USA), and Nitrocellulose (Grace Bio-Labs, Bend, Oreg.). Blocking formulations typically consist of blocking the surface prior to applying the plasma in an immunoassay, which by itself, is ineffective on the substrates tested when detecting antigen A and antigen B with anti-human IgG and anti-human IgM. Thus, a method to prevent undesired binding of the plasma constituents to the substrate surface will be advantageous.

In addition, prior to a transfusion, the blood group of the donor must be determined in order to properly match the blood with the patient that is receiving the blood. One of the tests used to make this determination is ABO reverse typing of the donor plasma, which detects anti-A and anti-B antibodies and confirms the ABO forward typing of the donor red blood cell antigens. Typically, reverse typing is done with a hemagglutination assay in a tube or a plate, requiring 2 tubes or 2 wells per sample for the test. Thus, a testing for anti-A and anti-B in one tube or sample well, requiring one tube or one well per sample, is highly desirable. It is even more desirable if both the IgM and IgG forms of these antibodies can be detected in one assay with satisfactory sensitivity. This is particularly advantageous for donors who have very low anti-A or anti-B that is IgM in nature.

### BRIEF SUMMARY

The inventive embodiments provided in this Brief Summary are meant to be illustrative only and to provide an overview of selective embodiments disclosed herein. The Brief Summary, being illustrative and selective, does not limit the scope of any claim, does not provide the entire scope of inventive embodiments disclosed or contemplated herein, and should not be construed as limiting or constraining the scope of this disclosure or any claimed inventive embodiment.

In certain embodiment, the disclosure encompasses a composition comprising a biological sample and an ethylene glycol (EG) based polymer having an average molecular weight of less than about 2000 dalton when dissolved in the biological sample. In certain embodiment, the EG based polymer has an average molecular weight of less than about 1000 dalton. In certain embodiment, the EG based polymer has an average molecular weight of less than about 800 dalton. In certain embodiment, the EG based polymer has an average molecular weight of less than about 600 dalton. In certain embodiment, the EG based polymer has an average molecular weight average of less than about 400 dalton. In certain embodiment, the EG based polymer is selected from the group consisting of a polyethylene glycol (PEG), tetraethylene glycol, a triethylene glycol, a diethylene glycol, an ethylene glycol monomer, and a mixture of any of the forgoing. In certain embodiment, the EG based polymer has one or more end groups selected from the group consisting of dimethyl ether, diglycidyl ether (diepoxy), and methyl ether. In certain embodiment, the EG based polymer is selected from the group consisting of tetraethylene glycol dimethyl ether, PEG dimethyl ether, PEG diglycidyl ether (diepoxy), PEG methyl ether, and a mixture of any of the forgoing.

In some embodiments, the biological sample of the composition comprises blood, serum, plasma, lymph fluid, bile fluid, urine, saliva, mucus, sputum, tears, cerebrospinal fluid (CSF), bronchioalveolar lavage, nasopharyngeal lavage, rectal lavage, vaginal lavage, colonic lavage, nasal lavage, throat lavage, synovial fluid, semen, ascites fluid, pus, maternal milk, ear fluid, sweat, and amniotic fluid.

In some embodiments, the composition of the present disclosure further comprising one or more solvents. In certain embodiment, the one or more solvent is water or PBS.

In certain embodiment, the EG based polymer of the composition has a concentration in the range of about 0.5 mg/ml to about 20 mg/ml. In certain embodiment, the EG based polymer has a concentration in the range of about 1.0 mg/ml to about 10 mg/ml.

In certain embodiment, the disclosure encompasses a non-fouling polymer layer comprising a brush polymer comprising a polymeric stem and a multitude of molecular bristles projecting from said polymeric stem, wherein the brush polymer comprises a co-polymer of an oligo ethylene glycol methacrylate (OEGMA) monomer and a methacrylate monomer (MAM) comprising a linking moiety and an electrophilic head group, wherein said co-polymer comprises a MAM to OEGMA v/v ratio from about 1:3 to about 1:8. In certain embodiment, the MAM to OEGMA v/v ratio is about 1:4. In certain embodiment, the OEGMA comprises poly(ethylene glycol) methacrylate (PEGMA) and poly(ethylene glycol) methyl ether methacrylate (PEGMEM). In certain embodiment, the electrophilic head group is an epoxide group or an epoxy-ketone group. In certain embodiment, the MAM is glycidyl methacrylate (GMA).

In certain embodiment, the co-polymer is epoxy-co-POEGMA. In certain embodiment, the co-polymer comprises GMA and PEGMEM, and wherein the GMA to PEGMEM ratio is about 1:4.

In certain embodiment, the disclosure encompasses a device comprising: (a) a substrate comprising a surface; (b) the non-fouling polymer layer of the present disclosure on the surface; and (c) one or more capture regions on the non-fouling polymer layer, comprising at least one capture agent.

In some embodiments, the device comprises a plurality of capture regions, wherein each capture region comprises at least one capture agent. In some embodiments, the plurality of capture regions comprise at least two, three, or four different capture agents. In some embodiments, each of the plurality of capture regions comprises a different capture agent. In some embodiments, the capture agent comprises a cell, a small molecule ligand, a lipid, a carbohydrate, a polynucleotide, a peptide, a protein, an antigen, or an antibody. In some embodiments, the origin of capture agent is human, humanized, murine, chimeric, or synthetic. In some embodiments, the substrate is glass, silicon, a metal oxide, or a polymer. In some embodiments, the device comprises one or more compartments. In some embodiments, wherein the device comprises a plurality of compartments.

In another embodiment, the disclosure encompasses a detector comprising: a body configured to accept the device of present disclosure; a lid which, in combination with the body, substantially surrounds the chip when the device is disposed in the body; a light source that is positioned to emit a light of a first wavelength such that the light contacts the non-fouling polymer layer; a filter that is positioned to filter light of a second wavelength emitted from the non-fouling polymer layer; a lens that is positioned to magnify a light of the second wavelength that passes through the filter; and a power source that provides power for the light source.

In some embodiments, the detector is a microarray detector or a nanoarray detector. In some embodiments, the detector has a volume of approximately 20-30 cm³. In some embodiments, the detector has a volume of about 25 cm³. In some embodiments, the detector is self-contained. In some embodiments, the detector is disposable.

In another embodiment, the disclosure encompasses a method of manufacturing a device, comprising: (a) providing a substrate comprising a surface; and (b) forming on the surface the non-fouling polymer layer of present disclosure. In some embodiments, the method further comprises printing at least one capture agent onto the non-fouling polymer layer. In some embodiments, the method further comprises printing a plurality of capture agents onto the non-fouling polymer layer. In some embodiments, the substrate used in the method is glass, silicon, a metal oxide, or a polymer.

In yet another embodiment, the disclosure encompasses a method for analyzing a biological sample comprising: (a) contacting the biological sample with an ethylene glycol (EG) based polymer having an average molecular weight of less than about 2000 dalton when dissolved in the biological sample, and (b) contacting the biological sample with a non-fouling polymer layer. In some embodiments, the EG based polymer of this method has an average molecular weight of less than about 1000 dalton. In some embodiments, the EG based polymer of this method has an average molecular weight of less than about 800 dalton. In some embodiments, the EG based polymer of this method has an average molecular weight of less than about 600 dalton. In some embodiments, the EG based polymer of this method an average molecular weight average of less than about 400 dalton. In some embodiments, the EG based polymer of this method is selected from the group consisting of a polyethylene glycol (PEG), tetraethylene glycol, a triethylene glycol, a diethylene glycol, an ethylene glycol monomer, and a mixture of any of the forgoing. In some embodiments, the EG based polymer of this method has one or more end groups selected from the group consisting of dimethyl ether, diglycidyl ether (diepoxy), and methyl ether. In some embodiments, the EG based polymer of this method is selected from the group consisting of tetraethylene glycol dimethyl ether, PEG dimethyl ether, PEG diglycidyl ether (diepoxy), PEG methyl ether, and a mixture of any of the forgoing. In some embodiments, the biological sample of this method comprises blood, serum, plasma, lymph fluid, bile fluid, urine, saliva, mucus, sputum, tears, cerebrospinal fluid (CSF), bronchioalveolar lavage, nasopharyngeal lavage, rectal lavage, vaginal lavage, colonic lavage, nasal lavage, throat lavage, synovial fluid, semen, ascites fluid, pus, maternal milk, ear fluid, sweat, and amniotic fluid. In some embodiments, the method further comprises one or more solvents. In some embodiments, the one or more solvent is water or PBS.

In some embodiments, the EG based polymer of this method has a concentration in the range of about 0.5 mg/ml to about 20 mg/ml. In some embodiments, the EG based polymer of this method has a concentration in the range of about 1.0 mg/ml to about 10 mg/ml. In some embodiments, the non-fouling polymer layer of this method comprises a brush polymer comprising a polymeric stem and a multitude of molecular bristles projecting from said polymeric stem, wherein the brush polymer comprises a co-polymer of an oligo ethylene glycol methacrylate (OEGMA) monomer and a methacrylate monomer (MAM) comprising a linking moiety and an electrophilic head group, wherein said co-polymer comprises a MAM to OEGMA v/v ratio from about 1:3 to about 1:8. In some embodiments, the MAM to OEGMA v/v ratio is about 1:4. In some embodiments, the OEGMA comprises poly(ethylene glycol) methacrylate (PEGMA) and poly(ethylene glycol) methyl ether methacrylate (PEGMEM). In some embodiments, the electrophilic head group is an epoxide group or an epoxy-ketone group.

In some embodiments, the MAM is glycidyl methacrylate (GMA). In some embodiments, the co-polymer is epoxy-co-POEGMA. In some embodiments, the co-polymer comprises GMA and PEGMEM, and wherein the GMA to PEGMEM ratio is about 1:4. In some embodiments, the non-fouling polymer layer further comprises one or more capture regions printed on the non-fouling polymer layer, comprising at least one capture agent. In some embodiments, the non-fouling polymer layer comprises a plurality of capture regions, wherein each capture region comprises at least one capture agent. In some embodiments, wherein the plurality of capture regions comprise at least two, three, or four different capture agents. In some embodiments, each of the plurality of capture regions comprises a different capture agent.

In some embodiments, the capture agent is selected from a cell, a small molecule ligand, a lipid, a carbohydrate, a polynucleotide, a peptide, a protein, an antigen, an antibody, and a combination thereof. In some embodiments, the origin of capture agent is human, humanized, murine, chimeric, or synthetic. In some embodiments, the antigen is selected from at least one blood type antigen, at least one platelet antigen, at least one infectious disease antigen, at least one human leukocyte antigen (HLA), and any combination thereof. In some embodiments, the at least one infectious disease antigen is selected from a human immune deficiency virus (HIV) antigen, a hepatitis B virus (HBV) antigen, a hepatitis C virus (HCV) antigen, a human T-lymphotropic virus (HTLV) antigen, a Treponema pallidum (TP) antigen, and any combination thereof.

In some embodiments, the at least one blood type antigen is selected from human A blood type antigen, a human B blood type antigen, a human AB blood type antigen, a human 0 blood type antigen, a human Rh factor antigen, a human MNS blood type antigen, a human P blood type antigen, a human P1PK blood type antigen, a human Lutheran blood type antigen, a human Kell blood type antigen, a human Lewis blood type antigen, a human Duffy blood type antigen, a human Kidd blood type antigen, a human Diego blood type antigen, a human Yt or Cartwright blood type antigen, a human Xg blood type antigen, a human Scianna blood type antigen, a human Dombrock blood type antigen, a human Colton blood type antigen, a human Landsteiner-Wiener blood type antigen, a human Chido/Rodgers blood type antigen, a human H blood type antigen, a human Hh/Bombay blood type antigen, a human Kx blood type antigen, a human Gerbich blood type antigen, a human Cromer blood type antigen, a human Knops blood type antigen, a human Indian blood type antigen, a human Ok blood type antigen, a human Raph blood type antigen, a human John Milton Hagen blood type antigen, a human I blood type antigen, a human li blood type antigen, a human Globoside blood type antigen, a human Gill blood type antigen, a human Rh-associated glycoprotein blood type antigen, a human Forssman blood type antigen, a human Langereis blood type antigen, a human Junior blood type antigen, and any combination thereof. In some embodiments, the antibody is selected from IgG, IgM, IgA, IgD, IgE, and any combination thereof.

In some embodiments, contacting the biological sample with an ethylene glycol (EG) based polymer and contacting the biological sample with a non-fouling polymer layer of the method occur sequentially or essentially simultaneously. In some embodiments, contacting the biological sample with an ethylene glycol (EG) based polymer occurs prior to contacting the biological sample with a non-fouling polymer layer. In some embodiments, contacting the biological sample with an ethylene glycol (EG) based polymer occurs post to contacting the biological sample with a non-fouling polymer layer. In some embodiments, the method further comprises contacting the biological sample with one or more detection agents. In some embodiments, the one or more detection agents comprise a first and a second detection agent. In some embodiments, the one or more detection agents comprise one or more detection moieties selected from a chromophore, a fluorophore, a biotin, a radiolabel, a polynucleotide, a small molecule, an enzyme, a nanoparticle, a microparticle, a quantum dot, or an upconverter.

In certain embodiment, the disclosure encompasses a kit comprising the composition of the present disclosure, a set of buffers and/or reagents, and instructions for use.

In another embodiment, the disclosure encompasses a kit comprising the device of the present disclosure, a set of buffers and/or reagents, and instructions for use.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls. The patents and patent applications incorporated herein by reference include at least U.S. Pat. Nos. 7,713,689, 8,367,314, US 20060057180, US 20090247424, US 20130157889, and US 20130143771.

### BRIEF DESCRIPTION OF THE DRAWINGS

FIG. 1 illustrates POEGMA synthesis scheme.

FIG. 2 illustrates exemplary non-reactive POEGMA.

FIG. 3 illustrates exemplary reactive surface: Epoxy-co-POEGMA.

FIG. 4 illustrates results from immuno-staining assay. Array of Mouse anti-Human IgM (anti-A and anti-B) was printed on each type of the e-POEGMA substrate plus a regular POEGMA as reference. Subsequently, the antibody arrays were stained with cy3-labeled Goat anti-Mouse IgM.

FIG. 5A and FIG. 5B illustrate IL-6 antigen titration results in duplicate from model assay conditions (error bar: +/−3σ). For each assay, dash-dotted lines are drawn at the intensity of the negative control (NC)+3 standard deviation. The lowest IL-6 antigen concentration that results intensity above NC+3 standard deviations is considered as the limit of detection (LoD). The dotted line indicates background for the blank.

FIG. 6A illustrates the IL-6 assay in human plasma and FIG. 6B illustrates the IL-6 assay in human serum (error bar: +/−3σ). The dotted lines are background intensity for the blank. The dash-dotted lines are drawn at the intensity of the blank (NC)+3 standard deviation. The lowest IL-6 antigen concentration that results intensity above NC+3 standard deviations is considered as the limit of detection (LoD).

FIG. 7A illustrates an exemplary double antigen bridge assay. FIG. 7B illustrates double antigen bridge assay for samples from Syphilis Accuset performance panel (0820-0214). Error bar: +/−σ.

FIG. 8A and FIG. 8B illustrate increased antibody retention in Epoxy-co-POEGMA. Different antibodies (anti-RBC 33F1, anti-A 120, anti-B 110, anti-D 401 and anti-D F8D8 tetramers) were spotted on epoxy versus regular POEGMA slides (with equivalent thickness). After 20 days of storage at RT, a subset of the arrays were treated with mock typing assays using PBS (no cells) on the BioChip Development Platform (BDP) 1.0, and an immunostaining protocol with fluorescently labeled secondary antibodies was performed to interrogate the presence (“retention”) of the printed antibodies. FIG. 8A shows the mean fluorescence intensity (MFI) analysis for arrays stained with anti-human IgM-Alexa Fluor 647 (AF647) (for detection of anti-D 401) and anti-mouse IgM-AF647 (for anti-A 120 and anti-B 110). PBS was used as a negative control for the staining procedure. The boxes highlight the spots with relevant expected signals. FIG. 8B shows the MFI analysis for arrays stained with anti-human IgG-AF555 (for detection of anti-D F8D8) and anti-mouse IgG-AF555 (for anti-RBC 33F1). PBS was used as a negative control for the staining procedure. The boxes highlight the spots with relevant expected signals. Anti-D F8D8 was printed as a tetramer containing mouse IgG anti-human IgG.

FIG. 9 illustrates that Epoxy-co-POEGMA and traditional (“regular”) POEGMA produce similar RBC typing assay results for the same donor cells. Different antibodies (anti-RBC 33F1, anti-A 120, anti-B 110, anti-D 401 and anti-D F8D8 tetramers) were spotted on epoxy versus regular POEGMA slides (all Abs printed at 0.5 mg/mL). RBC typing assays were performed using 3D printed 5.5 mm-wide coffin adapters on the BDP 1.0 using a ramp up wash step (from 30 to 150 μL/sec). Representative images are shown for 4 different in-house donors with different expected phenotypes.

FIG. 10 illustrates the comparison of fluorescent signal intensity across three donors' plasma detected using four different polyclonal anti hIgGs.

FIG. 11 illustrates the comparison of fluorescent signal intensity of two donors' plasma (X0137 and X0157) detected using polyclonal and monoclonal anti hIgG as well as anti hIgM.

FIG. 12 illustrates the direct immobilization of red cells on Epoxy-co-POEGMA and regular POEGMA through physical adsorption shows a more uniform spots on Epoxy-co-POEGMA. Cells spots are imaged after exposing the cells to plasma and detected with Alexa 647 labeled detection anti hIgG.

FIG. 13A illustrates the magnified images of indirect cells monolayer immobilized on the spot of anti RBC tetramer. Top: bright field images of immobilized red cells, bottom: cells are exposed to plasma and then labeled with Alexa 647 labeled anti hIgG. FIG. 13B illustrate the macrospot of red cells (0.5 μl) on microspot of anti RBC tetramer on the cooled substrate to the dew point (˜9° C.). Cells macrospot spread more on regular POEGMA compared to Epoxy-co-POEGMA.

FIG. 14 illustrates the comparison of anti D specificity (Max/Min signal or Max-Min/Max STD) determined based on the lowest concentration at which the signal intensity from the positive cells can be differentiated from the negative cells. The dotted line (LOD) represents equal signal intensity of the positive and negative cells/spots.

FIG. 15 illustrates the comparison of ABO reverse typing specificity with cells immobilized on Epoxy-co-POEGMA vs. regular POEGMA.

FIG. 16A illustrates the images of immobilized FSL-antigen on POEGMA substrate after ABO reverse typing shows significant difference in the adsorption of the FSL antigen on Epoxy vs. regular POEGMA. FIG. 16B illustrates that the more efficient adsorption of the FSL antigen on Epoxy-co-POEGMA resulted in a better retention and higher signal intensity of ABO reverse typing which leads to higher specificity (Positive cells RFU/Negative cells RFU) of the assay shown in FIG. 16C.

FIG. 17A illustrates the T7 probe intensity (error bar: +/−σ). FIG. 17B illustrates the T7 hybridization assay intensity.

FIG. 18A illustrates the comparison of IL-6 immunoassay results with commercial substrates (error bar: +/−3σ). Dotted line at blank (NC)+3 standard deviations indicates limit of detection cut-off. FIG. 18B illustrates the zoom-in of FIG. 18A at lower concentration range (0-10 pg/mL).

FIG. 19 illustrates the comparison of binding capacity (vertical axis in logarithmic scale).

FIG. 20 illustrates the nine varieties of PEG used in the experiment.

FIG. 21 illustrates the experimental layout for slide #1: Dosing different PEGs into the same plasma X0044.

FIG. 22 illustrates the fluorescence image of slide #1: Effect of dosing X0044 plasma with different PEGs.

FIG. 23 illustrates the plot of the mean fluorescence intensity measured for each well: background signal observed after pre-incubation of X0044 plasma with each of the PEGs listed.

FIG. 24 illustrates the experimental layout for slide #2: Different plasmas treated with tetraethylene glycol dimethyl ether in order to reduce the background signal.

FIG. 25 illustrates the fluorescence image of slide #2: Reduction of background signal from six plasmas by pre-incubating with tetraethylene glycol dimethyl ether.

FIG. 26 illustrates the X0172 plasma treated with different small molecular weight PEGs.

FIG. 27 illustrates the plot of the background signal, FSL-A signal, FSL-B signal, and specificity.

FIG. 28 illustrates the print layout for slide WRD125-7.

FIG. 29A illustrates the experimental layout for ePOEGMA slide 125-7. FIG. 29B illustrates the slide assembled with the FlexWell adapter, sealed with tape after the assay, turned on its side to allow the unbound cells to settle with gravity, leaving the bound cells on the array.

FIG. 30A illustrates darkfield microscopy images of wells that were exposed to one high titer A donor and one low titer A donor. FIG. 30B illustrates darkfield microscopy images of wells that were exposed to one medium titer B donor and one low titer B donor. FIG. 30C illustrates darkfield microscopy images of wells that were exposed to two AB donors. FIG. 30D illustrates darkfield microscopy images of wells that were exposed to one high titer O donor and one low hIgM titer O donor.

FIG. 31 illustrates the schematic of the double antigen cell bridge assay.

FIG. 32A illustrates photo of one version of the scattering imaging system. A light source is directed through a slit towards the slide, which is held in an upright position, avoiding re-dispersion of the unbound cells. A camera is positioned on the other side of the slide and captures the scattering exhibited by the cells bound to the array. FIG. 32B illustrates an additional version of the scattering imaging system, in which two beams are used. The light beams are directed through slits toward the slide, which is held upright in a slide holder. A diffuser with a mask is placed between the light source and the slide. The camera is positioned on the other side of the slide and captures the scattering from the cells bound to the array.

FIG. 33 illustrates the print layout for all of the slides used in this experiment. Scattering images and bright-field microscopy images were taken from different perspectives, but the 33F1 marker indicates the orientation of the FSL-antigen array.

FIG. 34 illustrates layout of the array in the wells. The array was printed on the left side of each well, so that it would be located in the upper half of the well once the slide was turned on its side. The last two wells in each slide, located over the barcode, were not used in this assay.

FIG. 35 illustrates a representative scattering image of wells 1-8 of slide WRD261-11.

FIG. 36 illustrates background-subtracted mean and median scattering intensity of 50 pM FSL-antigens exposed to Type A plasma donors. The mean and median values were very similar. The mean scattering intensity of the expected positive spot, FSL-B, was greater than or equal to 5000 for all of the donors. The signal for the expected negative spot, FSL-A, was less than 2500 for all donors, and less than 1000 for 17 out of 18 samples. A reference line at 2500 is shown in red.

FIG. 37 illustrates mean background-subtracted scattering intensity of 50 pM FSL-antigens exposed to Type B donors. The signal for the expected positive spot, FSL-A, was greater than 5000 for all of the donors. The signal for the expected negative spot, FSL-B, was less than 2500 for all donors, and less than 1000 for 14 out of 15 samples. A reference line at 2500 is shown in red.

FIG. 38 illustrates mean scattering intensity of 50 pM FSL-antigens exposed to Type AB plasma donors. The average signal for all of the spots was below 1000.

FIG. 39 illustrates mean scattering intensity of 50 pM FSL-antigens exposed to Type O plasma donors. While most of the spots exhibited signals greater than 5000, a few of the spots had signals close to or below 2500. A reference line at 2500 is shown in red.

FIG. 40 illustrates plot of the mean scattering intensity for all of the donors separated based on blood type. Data for both the 15 pM and the 50 pM FSL-antigen spots are included. Reference lines at 0, 1000, and 2500 are shown in black, red, and green, respectively.

FIG. 41 illustrates scattering images of the four wells exposed to donor X0044, a high titer Type A donor. The print layout is shown on the left. Although there was no obvious non-specific binding to the FSL-A spots, a streak was observed down the column of the 15 pM FSL-A spots on slide WRD261-1. Some differences in intensity from slide to slide were also observed for both the positive spots and the background.

FIG. 42 illustrates quantified data for wells exposed to donor X0044 on four different slides for the 15 pM and the 50 pM FSL-antigen spots. The scattering signal from the negative spots was very low with the exception of the 15 pM FSL-A column on slide WRD261-1 where the streak was observed in the scattering image. The intensity values for the FSL-B positive spots were similar from slide to slide with the exception of WRD261-11, which was approximately ⅔ of the intensity of the rest.

FIG. 43 illustrates bright-field microscopy images of the first row of wells exposed to Type A donor X0044 on four different slides. The binding to FSL-B spots did not appear significantly different between slides. The smear seen in the scattering image for slide WRD261-1 was also visible in the microscope image.

FIG. 44 illustrates plots of the mean scattering intensity values for the 15 pM vs the 50 pM printed FSL-antigen spots for Type A and Type B donors. There was no correlation between the two concentrations for the negative spots, but there was some correlation between the two concentrations for the positive spots.

FIG. 45 illustrates plots of the mean scattering intensity values for the 15 pM vs the 50 pM printed FSL-antigen spots for Type AB and Type O donors. There was no correlation between the two concentrations for the negative spots, but there was some correlation between the two concentrations for the positive spots.

FIG. 46 illustrates linear regression analysis for the lowest intensity spots suggests that the relationship between 15 pM and 50 pM FSL-antigen spots is different than when the samples are taken as a whole, particularly for FSL-B.

FIG. 47 illustrates scattering and bright-field microscopy images of the array exposed to donor R0298 (B). Although the 50 pM FSL-B spots had a scattering intensity value above 1000, no cells were bound to the spots, suggesting that the high scattering may be a result of the imaging technique or the data analysis method.

FIG. 48 illustrates scattering and bright-field microscopy images of the well exposed to Type A donor R0121, showing sparse but localized binding of cells to the negative FSL-A spots.

FIG. 49 illustrates ABO reverse typing of donor R0121, showing no reaction with A1 or A2 cells and a strong reaction with B cells.

FIG. 50 illustrates scattering images of the well exposed to donor R0121 (A), where the FSL-A spots are expected to be negative, and the well exposed to donor W0145 (O), where the FSL-A spots are expected to be positive. The FSL-A spots cannot be distinguished from each other, leading to the mistyping of one of these donors, depending on what cutoff value is selected.

FIGS. 51A and 51B illustrate scatter er plot of FSL-A vs FSL-B (51A) and log(FSL-A) vs log(FSL-B) (51B) for the 15 pM FSL-antigens spots, showing four distinct clusters corresponding to the four blood groups. Reference lines at 1000 and 2500 are shown in red and blue, respectively.

FIGS. 52A and 52B illustrate scatter plot of FSL-A vs FSL-B (52A) and log(FSL-A) vs log(FSL-B) (52B) for the 50 pM FSL-antigens spots, showing four distinct clusters corresponding to the four blood groups. Reference lines at 1000 and 2500 are shown in red and blue, respectively

FIG. 53 illustrates contingency tables for 15 pM and 50 pM FSL-A and FSL-B generated using a cutoff value of 1000, and the percent agreement at the lower bound of the 95% confidence interval calculated from the tables.

FIG. 54 illustrates contingency tables for 15 pM and 50 pM FSL-A and FSL-B generated using a cutoff value of 2500, and the percent agreement at the lower bound of the 95% confidence interval calculated from the tables.

FIG. 55 illustrates contingency tables for 15 pM and 50 pM FSL-A and FSL-B generated using visual interpretation of the microscope images, and the percent agreement at the lower bound of the 95% confidence interval calculated from the tables. Both print concentrations generated the same tables.

FIG. 56 illustrates best point estimate for each scenario using the LaPlace method.

FIG. 57 illustrates boxplot of mean scattering intensity vs IgM titer for all samples expected to be positive for anti-A (Type B and Type O donors). In general, there was an increase in intensity with increased anti-A titer.

FIG. 58 illustrates boxplot of mean scattering intensity vs IgM titer for all samples expected to be positive for anti-B (Type A and Type O donors). In general, there was an increase in intensity with increased anti-B titer but there was a slight drop at the high end. An outlier, donor X0044 run on slide WRD261-11, was identified by Minitab (see discussion on X0044 reproducibility).

FIG. 59A and FIG. 59B show the flowcharts of steps used in a two-step and one-step labeling assays, respectively. FIG. 59C illustrates the principle of the antigen bridge assay.

FIG. 60A shows the samples used in the study (indicated by arrows), selected from SeraCare AccuSet™ Syphilis Performance panel (SeraCare Life Sciences, Gaithersburg, Md.).

FIG. 60B shows an exemplary array layout.

FIGS. 61A and 61B show the scan result and the quantitative results of Tp One-step Antigen-bridge Assay for samples Tp02, Tp04, Tp05, and Tp13.

FIG. 62 shows a comparison of biotin labeled Ag (biotin-Ag, 0.5 μg/ml) versus AF647-Ag (1 μg/ml). The three bars from left to right are R01549-AF647DOL12, R01549-AF647DOL8, and R01549-Biotin, respectively.

FIG. 63A and FIG. 63B show the scan result and the quantitative results of Tp One-step Antigen-bridge Assay for samples 12 and 18 at concentrations of 5, 1, 0.5, and 0.1 μg/mg for 10 or 30 minutes incubation time.

FIG. 64 shows an exemplary result for non-specific signals from CMV spots for each titration.

FIG. 65A and FIG. 65B show the scan result and the quantitative results of Tp One-step Antigen-bridge Assay for samples 02, 04, 05, and 12 and R01549-AF647DOL8 with holding time of 0, 5, 10, and 15 minutes.

FIGS. 66A-66C show the results of seroconversion panel test.

### DETAILED DESCRIPTION

### I. Definitions

Unless otherwise defined, all technical terms used herein have the same meaning as commonly understood by one of ordinary skill in the art to which this disclosure belongs.

As used herein, unless otherwise indicated, open terms such as “contain,” “containing,” “include,” “including,” and the like mean “comprising.”

“OEGMA” as used herein refers to oligo(ethylene glycol)methyl methacrylate.

The term “polymer,” as used herein, is given its ordinary meaning as used in the art, i.e., a molecular structure featuring one or more repeat units (monomers), connected by covalent bonds. The repeat units can all be identical, or in some cases, there can be more than one type of repeat unit present within the polymer. The term polymer is intended to encompass any type of polymer, including homopolymers, copolymers (e.g., random copolymers, block copolymers, graft copolymers, etc.), and blends, combinations and mixtures thereof. Polymers can be linear, branched, star-shaped, etc.

“Analyte” as used herein can be any second member of a specific binding pair. Typically the analyte is a constituent of, or found in, a sample such as a biological fluid. The analyte can be a biomarker as described below. In some of the embodiments described herein, an analyte is or comprises an antigen. In some of the embodiments described herein, an analyte is or comprises a human A blood type antigen, a human B blood type antigen, a human AB blood type antigen, a human 0 blood type antigen, a human Rh factor antigen, a human MNS blood type antigen, a human P blood type antigen, a human P1PK blood type antigen, a human Lutheran blood type antigen, a human Kell blood type antigen, a human Lewis blood type antigen, a human Duffy blood type antigen, a human Kidd blood type antigen, a human Diego blood type antigen, a human Yt or Cartwright blood type antigen, a human Xg blood type antigen, a human Scianna blood type antigen, a human Dombrock blood type antigen, a human Colton blood type antigen, a human Landsteiner-Wiener blood type antigen, a human Chido/Rodgers blood type antigen, a human H blood type antigen, a human Hh/Bombay blood type antigen, a human Kx blood type antigen, a human Gerbich blood type antigen, a human Cromer blood type antigen, a human Knops blood type antigen, a human Indian blood type antigen, a human Ok blood type antigen, a human Raph blood type antigen, a human John Milton Hagen blood type antigen, a human I blood type antigen, a human li blood type antigen, a human Globoside blood type antigen, a human Gill blood type antigen, a human Rh-associated glycoprotein blood type antigen, a human Forssman blood type antigen, a human Langereis blood type antigen, a human Junior blood type antigen, or any combination thereof.

As used herein, the term “sample” or “biological sample” relates to any material that is taken from its native or natural state, so as to facilitate any desirable manipulation or further processing and/or modification. A sample or a biological sample can comprise a cell, a tissue, a fluid (e.g., a biological fluid), a protein (e.g., antibody, enzyme, soluble protein, insoluble protein), a polynucleotide (e.g., RNA, DNA), a membrane preparation, and the like, that can optionally be further isolated and/or purified from its native or natural state.

As used herein, the term “biological fluid” refers to any a fluid originating from a biological organism. Exemplary biological fluids can include, but are not limited to, blood, serum, plasma, lymph fluid, bile fluid, urine, saliva, mucus, sputum, tears, cerebrospinal fluid (CSF), bronchioalveolar lavage, nasopharyngeal lavage, rectal lavage, vaginal lavage, colonic lavage, nasal lavage, throat lavage, synovial fluid, semen, ascites fluid, pus, maternal milk, ear fluid, sweat, and amniotic fluid. A biological fluid can be in its natural state or in a modified state by the addition of components such as reagents, or removal of one or more natural constituents (e.g., blood plasma). A sample or biological sample can be, for example, blood, plasma, lymph, viral, bacterial, a human sample, a diseased human sample, an animal sample, a disease animal sample, saliva, mucus, cerebral spinal fluid, synovial fluid, stomach fluid, intestinal fluid, cytoplasmic fluid, or other type of sample.

As used herein, the term “infectious disease” (herein abbreviated as ID) refers to those diseases that are caused by infectious agents including, but not limited to, microbes such as viruses, bacteria, archaea, planaria, amoeba, and fungi.

As used herein, the term “region” refers to a defined area on the surface of a material. A region can be identified and bounded by a distinct interface between two materials having different compositions.

“Specific binding pair” as used herein refers to two molecules that exhibit specific binding to one another, or increased binding to one another relative to other molecules. A specific binding pair can exhibit functional binding activity such as a receptor and a ligand (such as a drug, protein, or carbohydrate), an antibody and an antigen, etc.; or structural binding activity such as protein/peptide and protein/peptide; protein/peptide and nucleic acid; and nucleotide and nucleotide etc. Typically, one member of the binding pair can serve as a capture agent in the devices described herein, and the capture agent can bind to the second member of the binding pair, which can be present as an analyte in a sample such as a biological fluid.

The term “polypeptide” as used herein are art-recognized terms and are understood to refer to any polymer comprising a linear chain of amino acids, or amino acid analogs, regardless of its size or function. The term “polypeptide” as used herein refers to an oligopeptide, dipeptide, tripeptide, peptide, and/or polypeptide.

The term “protein” as used herein are art-recognized terms and are understood to refer to biomolecules consisting of one or more polypeptide molecules, regardless of its size or function. As used herein, the terms “protein,” “polypeptide,” and “peptide” are used interchangeably. In some embodiments, a protein is an antibody.

The terms “antibody” or “antibodies” as used herein are art-recognized terms and are understood to refer to molecules or active fragments of molecules that bind to known antigens, particularly to immunoglobulin molecules and to immunologically active portions of immunoglobulin molecules, i.e., molecules that contain a binding site that specifically binds an antigen. An immunoglobulin is a protein comprising one or more polypeptides substantially encoded by the immunoglobulin kappa and lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Also subclasses of the heavy chain are known. For example, IgG heavy chains in humans can be any of IgG1, IgG2, IgG3, and IgG4 subclass. The immunoglobulin according to the disclosure can be of any class (IgG, IgM, IgD, IgE, IgA, and IgY) or subclass (IgG1, IgG2, IgG3, IgG4, IgA1, and IgA2) of immunoglobulin molecule.

The term “capture agent” as used herein refers to a composition that comprises one or more target-binding moieties and which specifically binds to a target protein, peptide, or nucleotide (such as DNA or RNA), etc., via those target-binding moieties. Each target-binding moiety exhibits binding affinity for the target protein, either individually or in combination with other target-binding moieties. In certain embodiments, each target-binding moiety binds to the target protein via one or more non-covalent interactions, including for example hydrogen bonds, hydrophobic interactions, and van der Waals interactions. A capture agent may comprise one or more organic molecules, including, for example, polypeptides, proteins, antibodies, antigens, peptides, polynucleotides, and other non-polymeric molecules.

As used herein, the term “subject” and “patient” are used interchangeably and refer to both human and nonhuman animals. The term “nonhuman animals” can include all vertebrates, e.g., mammals and non-mammals, including but not limited to nonhuman primates, sheep, dog, cat, horse, cow, chickens, amphibians, reptiles, and the like. In certain embodiments, the subject is a human patient.

The term “detecting” as used herein refers to a method, step, or process of verifying the presence or absence of a given molecule. For example, detecting an antibody in a biological fluid can mean detecting the presence of an antibody in the biological fluid or detecting its absence. The detection may also be quantitative, i.e. include correlating the detected signal with the amount of analyte. The detection includes in vitro as well as in vivo detection.

As used herein, unless otherwise indicated, some embodiments herein contemplate numerical ranges. When a numerical range is provided, the range includes the range endpoints. Numerical ranges include all values and subranges therein as if explicitly written out.

The term “about” can refer to plus or minus 10% of a referenced numeric indication.

Articles “a,” “an,” and “the” are used herein to refer to one or to more than one (i.e. at least one) of the grammatical object of the article.

The term “and/or” as used herein should be understood to include any single element recited within the relevant phrase, as well as any combination of two or more elements, including all elements.

The term “essentially simultaneously” as used herein refers to two or more events or steps happening, existing, or done at the same time or with short intermission in between. In some embodiments, essentially simultaneously means at the same time. In some embodiments, essentially simultaneously means adding in short succession. In some embodiments, essentially simultaneously means immediately after. In some embodiments, essentially simultaneously means performing steps without washing in between, or first removing a biological fluid sample.

As used herein, a disposable detector means a detector adapted for a one-time use and disposed of after use. In certain embodiment, one or more of surface parts of detector is disposable and other parts of the detector may be re-used.

As used herein, a BioChip Development Platform (BDP) is a custom test system designed for the purpose of evaluating assay bioprocessing steps including wash and protein binding protocols in various BioChip configurations under repeatable and controlled conditions.

The details of one or more inventive embodiments are set forth in the accompanying drawings, the claims, and the description herein. Other features, objects, and advantages of the inventive embodiments disclosed and contemplated herein can be combined with any other embodiment unless explicitly excluded.

### II. Compositions

The present disclosure involves preventing background binding to the surface of a substrate by the addition of an inhibitor to the plasma, such that the binding sites of the plasma constituents that would bind to the surface are saturated, and thus unavailable for interaction with the surface. Higher molecular weight PEGs are typically used to precipitate immunoglobulins or other proteins prior to an assay, but that technique is unfavorable when the analyte of interest may precipitate as well. This disclosure does not block the surface, nor does it cause any precipitation. It instead acts as an inhibitor to unexpected binding of plasma to a surface, when added to plasma.

The present disclosure provides that dosing small molecular weight poly(ethylene glycol) molecules (molecular weight <1000) into plasma inhibits unexpected binding of plasma constituents to the substrate surface. In one embodiment, the process involves mixing a commercially available product (small molecular weight PEG with molecular weight <1000) with plasma. In one embodiment, the present disclosure provides a composition comprising a biological sample (e.g., a biological fluid) and an ethylene glycol (EG) based polymer having an average molecular weight of less than about 6000, 2000, 1000, 800, 600, or 400 dalton when dissolved in the biological sample.

In one embodiment, several small molecular weight PEG molecules such as tetraethylene glycol dimethyl ether, poly(ethylene glycol) methyl ether, and poly(ethylene glycol) diglycidyl ether can be added to several (e.g., six) different donor plasmas at a concentration of about 10 mg/ml, and the mixtures can be used in an immunoassay to detect IgM antibody binding to the substrate. When compared with the same plasmas that are not dosed, there can be up to a 90-fold reduction in background signal on our in-house epoxy POEGMA substrate and a 2-5-fold reduction in background signal on a commercially available nitrocellulose substrate.

### III. Substrates

In one embodiment, the present disclosure provides an optimized surface chemistry to allow optimum protein microarray adhesion and assay detection for immunoassay development. In another embodiment, the present disclosure provides a surface that can retain proteins that are spotted to form a microarray and repel non-specific protein adhesion during assay. In one embodiment, the ePOEGMA surface of the present disclosure has this property due to its nano-roughness.

The present disclosure can be utilized to form surfaces on a variety of different types of substrates.

In some embodiments, the article is a label-free optical or mass detector (e.g., a surface plasmon resonance energy detector, an optical wave guide, an ellipsometry detector, etc.) and the surface is a sensing surface (e.g., a surface portion that would be in contact with a biological fluid). Examples of such articles include but are not limited to those described in U.S. Pat. Nos. 6,579,721; 6,573,107; 6,570,657; 6,423,055; 5,991,048; 5,822,073; 5,815,278; 5,625,455; 5,485,277; 5,415,842; 4,844,613; and 4,822,135.

In other embodiments, the article is a biosensor, an assay plate, or the like. For example, the present disclosure may be utilized with optical biosensors such as described in U.S. Pat. No. 5,313,264 to Ulf et al., U.S. Pat. No. 5,846,842 to Herron et al., U.S. Pat. No. 5,496,701 to Pollard-Knight et al., etc. The present disclosure may be utilized with potentiometric or electrochemical biosensors, such as described in U.S. Pat. No. 5,413,690 to Kost, or PCT Application WO98/35232 to Fowlkes and Thorp. The present disclosure may be utilized with a diamond film biosensor, such as described in U.S. Pat. No. 5,777,372. Thus, the solid support may be organic or inorganic; may be metal (e.g., copper or silver) or non-metal; may be a polymer or nonpolymer; may be conducting, semiconducting or nonconducting (insulating); may be reflecting or nonreflecting; may be porous or nonporous; etc. For example, the solid support may be comprised of polyethylene, polytetrafluoroethylene, polystyrene, polyethylene terephthalate, polycarbonate, gold, silicon, silicon oxide, silicon oxynitride, indium, tantalum oxide, niobium oxide, titanium, titanium oxide, platinum, iridium, indium tin oxide, diamond or diamond-like film, etc.

The present disclosure may be utilized with substrates for “chip-based” and “pin-based” combinatorial chemistry techniques. All can be prepared in accordance with known techniques. See. e.g., U.S. Pat. No. 5,445,934 to Fodor et al., U.S. Pat. No. 5,288,514 to Ellman, and U.S. Pat. No. 5,624,711 to Sundberg et al., the disclosures of which are incorporated by reference herein in their entirety.

Substrates as described above can be formed of any suitable material, including but not limited to a material selected from the group consisting of metals, metal oxides, semiconductors, polymers (particularly organic polymers in any suitable form including woven, nonwoven, molded, extruded, cast, etc.), silicon, silicon oxide, and composites thereof.

Polymers used to form substrates as described herein may be any suitable polymer, including but not limited to: poly(ethylene) (PE), poly(propylene) (PP), cis and trans isomers of poly(butadiene) (PB), cis and trans isomers of poly(ispoprene), poly(ethylene terephthalate) (PET), polystyrene (PS), polycarbonate (PC), poly(epsilon-caprolactone) (PECL or PCL), poly(methyl methacrylate) (PMMA) and its homologs, poly(methyl acrylate) and its homologs, poly(lactic acid) (PLA), poly(glycolic acid), polyorthoesters, poly(anhydrides), nylon, polyimides, polydimethylsiloxane (PDMS), polybutadiene (PB), polyvinyl alcohol (PVA), polyacrylamide and its homologs such as poly(N-isopropyl acrylamide), fluorinated polyacrylate (PFOA), poly(ethylene-butylene) (PEB), poly(styrene-acrylonitrile) (SAN), polytetrafluoroethylene (PTFE) and its derivatives, polyolefin plastomers, and combinations and copolymers thereof, etc.

If desired or necessary, the substrate may have an additional layer such as a gold or an oxide layer formed on the relevant surface portion to facilitate the deposition of the linking layer.

In certain embodiments, the POEGMA substrate is poly(ethylene glycol) methyl ether methacrylate (PEGMEM, e.g., with an average molecular weight of about 300). In certain embodiments, the POEGMA substrate is poly(ethylene glycol) methacrylate (PEGMA, e.g., with an average molecular weight of about 360). In certain embodiments, the e-POEGMA substrate is a mixture of PEGMEM and glycidyl methacrylate (GMA, with an average molecular weight of about 142).

### IV. Devices

Substrates for use in the present disclosure can be in the form of a chip or an array, such as a microarray. Typically, a chip of the disclosure will define a channel that extends at least partially into the interior of the chip. The channel may have one or more non-fouling polymer layers disposed on one or more of the channel surfaces. The channel may be open at one or both ends and is generally covered so as to form a tube that extends through the chip. In some embodiments, a chip of the disclosure may rely upon capillary action to draw a sample (e.g., a biological fluid such as blood) into the channel. Thus, a channel may have dimensions that support capillary action. In other embodiments, a chip of the disclosure may define an open well. In other embodiments, the device may have closed well. In certain embodiment, the closed well device can have improved bound/free separation by creating a flow across the device, e.g., a chip or an array. In other embodiments, the device may be quasi-microfluidic. In certain embodiment, the quasi-microfluidic device can have reduced variability of flow across the device, e.g., a chip or an array. In one embodiment, the chip is designed with a channel to accept blood from a fingerstick via capillary action. In such embodiments, the channel may be designed to hold volumes of a few microliters, for example, from about 0.5 microliter to about 300 microliters, from about 0.5 microliter to about 250 microliters, from about 0.5 microliter to about 200 microliters, from about 0.5 microliter to about 150 microliters, from about 0.5 microliter to about 100 microliters, from about 0.5 microliter to about 75 microliters, from about 0.5 microliter to about 50 microliters, from about 0.5 microliter to about 25 microliters, from about 0.5 microliter to about 10 microliter, from about 0.5 microliter to about 5 microliters, from about 1 microliter to about 100 microliters, from about 1 microliter to about 75 microliters, from about 1 microliter to about 50 microliters, from about 1 microliter to about 25 microliters, from about 1 microliter to about 10 microliter, from about 1 microliter to about 5 microliters, from about 2.5 microliters to about 100 microliters, from about 2.5 microliters to about 75 microliters, from about 2.5 microliters to about 50 microliters, from about 2.5 microliters to about 25 microliters, from about 2.5 microliters to about 10 microliter, or from about 2.5 microliters to about 5 microliters. In one embodiment, a chip of the disclosure may define a channel that has dimensions of approximately 4 mm wide, by 9 mm long, by 0.1 mm high (3.6 microliters). Typically, one or more surface of the channel will comprise one or more micro- or nano-spots. The spots may comprise one or more reagents that will be used in performance of assays of the disclosure. In one embodiment, spots may be used that are about 100 microns in diameter.

In one embodiment, a chip of the disclosure defines a channel having the dimensions recited above. On the bottom surface of the channel 100 micron diameter spots are disposed. The spots may have a center to center spacing of 200 microns. In embodiments of this type, a 4 mm×9 mm channel could hold roughly 900 of the 100 micron diameter spots. The channel dimensions, spot size, and/or spot spacing can be adjusted so as to accommodate a desired number of spots. A suitable number of spots may be from about 100 to about 10000 spots, from about 100 to about 7500 spots, from about 100 to about 5000 spots, from about 100 to about 2500 spots, from about 100 to about 1000 spots, 500 to about 10000 spots, from about 500 to about 7500 spots, from about 500 to about 5000 spots, from about 500 to about 2500 spots, or from about 500 to about 1000 spots. Each spot may be a different material, although duplicate spots are generally desirable for reproducibility.

In some embodiments, a chip of the disclosure may comprise one or more dams. Dams may be provided to separate one or more spots from one or more other spots. Dams may be water soluble and made out of any material known to those skilled in the art. Dams may be disposed on the chip between the capture agent and a detection agent. Dams may comprises a water-soluble salt, water-soluble sugar, a water-soluble polymer, or any combination thereof. Suitable examples of materials from which a dam may be constructed include, but are not limited to, a phosphate salt, a citrate salt, trehalose, polyvinyl alcohol, polyethylene glycol, or any combination thereof.

A dam may be disposed at any position on the channel of a chip. For example, a dam may be placed at the fluid entrance of the channel, at a point within the channel or at the end of the channel opposite the fluid entrance of the channel. A dam may be disposed across all or a portion of the width of the channel. In some embodiments, a chip may define a channel comprising a plurality of spots and also comprising a dam across the width of the channel.

A chip of the disclosure may be made using two glass coverslips separated by double sided tape to make a space between the chips thereby defining a channel. In assays of the disclosure that employ optical detection methods, e.g., fluorescence detection, any optically clear material could be used as substrate, including plastics.

In some embodiments, the device (e.g., a chip or an array) of the disclosure may comprise a chamber (e.g., a reaction chamber). In some embodiments, the chamber has an elongated shape. In some embodiments, the chamber has two opening, for example, an inlet and an outlet. In some embodiment, the two openings are located on the two distal ends. In some embodiments, the chamber has a height of about 1 mm to 100 μm, about 800 μm, about 600 μm, about 500 μm, about 400 μm, about 300 μm, about 200 μm, about 170 μm, about 150 μm, about 100 μm, or about 50 μm. In some embodiments, the chamber has a width of about 1-5 mm, about 4 mm, about 3 mm, about 2.5 mm, about 2.3 mm, about 2 mm, or about 1 mm. In some embodiments, the chamber has a length of about 5 mm to about 15 mm, about 10 mm, about 9 mm, about 8 mm, or about 7 mm. In some embodiments, the chamber has a square print array. In some embodiments, the chamber has a rectangular print array. In some embodiments, the chamber has one print array. In some embodiments, the chamber has more than one print array.

### V. Linking Layer

Depending on the choice of substrate and polymer, a linking layer can optionally be included between the substrate and the polymer layer. For example, a linking layer can be formed from a compound comprising an anchor group coupled (e.g., covalently coupled) to an initiator (e.g., directly coupled or coupled through an intermediate linking group). The choice of anchor group can depend upon the substrate on which the linking layer is formed, and the choice of initiator can depend upon the particular reaction used to form the non-fouling polymer as discussed in greater detail below.

The anchoring group can covalently or non-covalently couple the compound or linking layer to the surface of the substrate. Non-covalent coupling can be by any suitable secondary interaction, including but not limited to hydrophobic interactions, hydrogen bonding, van der Waals forces, ionic bonds, metal-ligand interactions, etc.

Examples of substrate materials and corresponding anchoring groups can include, for example, gold, silver, copper, cadmium, zinc, palladium, platinum, mercury, lead, iron, chromium, manganese, tungsten, and any alloys thereof with sulfur-containing functional groups such as thiols, sulfides, disulfides (e.g., —SR or —SSR where R is H, alkyl such as lower alkyl, or aryl), and the like; doped or undoped silicon with silanes and chlorosilanes (e.g., —SiR₂Cl wherein R is H, alkyl such as lower alkyl, or aryl); metal oxides such as silica, alumina, quartz, glass, and the like with carboxylic acids as anchoring groups; platinum and palladium with nitrites and isonitriles; and copper with hydroxamic acids. Additional suitable functional groups suitable as the anchoring group can include benzophenones, acid chlorides, anhydrides, epoxides, sulfonyl groups, phosphoryl groups, hydroxyl groups, phosphonates, phosphonic acids, amino acid groups, amides, and the like. See, e.g., U.S. Pat. No. 6,413,587.

Any suitable initiator can be incorporated into the anchoring group by introduction of a covalent bond at a location non-critical for the activity of the initiator. Examples of such initiators can include, but are not limited to, bromoisobutyrate, polymethyl methacrylate-Cl, polystyrene-Cl, AIBN, 2-bromoisobutyrate, chlorobenzene, hexabromomethyl benzene, hexachloromethyl benzene, dibromoxylene, methyl bromoproprionate. Additional examples of initiators can include those initiators described in U.S. Pat. No. 6,413,587 (e.g., at columns 10-11 thereof) and those initiators described in U.S. Pat. No. 6,541,580.

As noted above, a linking group or “spacer” can be inserted between the anchoring group and initiator. The linker can be polar, nonpolar, positively charged, negatively charged or uncharged, and can be, for example, saturated or unsaturated, linear or branched alkylene, heteroalkylene, aralkylene, alkarylene, or other hydrocarbylene, such as halogenated

hydrocarbylene, particularly fluorinated hydrocarbylene. Suitable linkers can be saturated alkylene groups of 3 to 20 carbon atoms, i.e., —(CH₂)ₙ—, where n is an integer of 3 to 20 inclusive. See, e.g., U.S. Pat. No. 6,413,587. Another suitable embodiment of the linker is an oligoethyleneglycol of 3 to 20 units, i.e., —(CH₂CH₂O)ₙ— where n is an integer of 3 to 20 inclusive.

The anchoring layer can be deposited by any suitable technique. It can be deposited as a self-assembled monolayer. It can be created by modification of the substrate by chemical reaction (see, e.g., U.S. Pat. No. 6,444,254) or by reactive plasma etching or corona discharge treatment. It can be deposited by a plasma deposition process. It can be deposited by spin coating or dip coating. It can be deposited by spray painting. It can also be deposited by deposition, printing, stamping, etc. It can be deposited as a continuous layer or as a discontinuous (e.g., patterned) layer.

In some of the embodiments described herein, the substrate can be glass (such as slide, plate or wafer, or lase scribed pre-scored glass), silicon oxide or other inorganic or semiconductor material (e.g., silicon oxide, silicon nitride) or compound semiconductors (e.g., gallium arsenide, and indium gallium arsenide) commonly used for microarray production. In some of the embodiments described herein, the substrate can be a microtiter (microwell) plate.

In some of the embodiments described herein, the anchoring group can be a silane or chlorosilane (e.g., —SiR₂Cl wherein R is H, alkyl such as lower alkyl, or aryl).

In some of the embodiments described herein, the linking layer is formed on the substrate in two separate steps. For example, in a first step, an anchoring layer of alkyl silane or alkanethiol can be deposited on a surface such as silicon dioxide or glass or gold, and presents a terminal reactive functional group (e.g., amine) Subsequently, a bifunctional molecule, which comprises a first functional group reactive towards the terminal group presented by the first linking layer can be reacted with the first linking layer deposited in the first step. The second functional group of the bifunctional molecule contains a moiety group that acts as an initiator for the polymerization of the polymer layer, such as an ATRP initiator.

### VI. Polymer Layer

The polymer layers of the devices described herein exhibit non-fouling properties. Non-fouling, as used herein with respect to the polymer layer, relates to the inhibition (e.g., reduction or prevention) of growth of an organism as well as to non-specific or adventitious binding interactions between the polymer and an organism or biomolecule (e.g., cell, protein, nucleotide, etc.). The non-fouling property of the polymer can be introduced by any suitable method such as incorporation of a non-fouling (or alternatively, antifouling) agent or by the structure/architecture of the polymer itself Non-fouling agents are known in the art and can be selected by one of skill depending on the particular use of device, or on the availability of the non-fouling agent. Non-limiting examples can include organic and inorganic compounds having biocidal activity, as well as compounds that can be incorporated with or bound to the polymer layer that reduce or inhibit non-specific binding interaction of a biomolecule (e.g., cell, protein, nucleotide, carbohydrate/lipid) with the polymer upon contact.

Some embodiments provide a polymer layer having a structure or architecture that provides a non-fouling property. In some of the embodiments described herein, the polymer can suitably include brush polymers, which are, in general, formed by the polymerization of monomelic core groups having one or more groups that function to inhibit binding of a biomolecule (e.g., cell, protein, nucleotide, carbohydrate/lipid) coupled thereto. Suitably, the monomelic core group can be coupled to a protein-resistant head group.

Polymer layers can suitably be formed using radical polymerization techniques, such as catalytic chain transfer polymerization, iniferter mediated polymerization (e.g., photoiniferter mediated polymerization), free radical polymerization, stable free radical mediated polymerization (SFRP), atom transfer radical polymerization (ATRP), and reversible addition-fragmentation chain transfer (RAFT) polymerization. For example, free radical polymerization of monomers to form brush polymers can be carried out in accordance with known techniques, such as described in U.S. Pat. Nos. 6,423,465, 6,413,587 and 6,649,138, U.S. Patent Application No. 2003/0108879, and variations thereof which will be apparent to those skilled in the art.

Atom transfer radical polymerization of monomers to form brush polymers can also be carried out in accordance with known techniques, such as described in U.S. Pat. Nos. 6,541,580 and 6,512,060, U.S. Patent Application No. 2003/0185741, and variations thereof which will be apparent to those skilled in the art.

Any suitable core vinyl monomer polymerizable by the processes discussed above can be used, including but not limited to styrenes, acrylonitriles, acetates, acrylates, methacrylates, acrylamides, methacrylamides, vinyl alcohols, vinyl acids, and combinations thereof.

In some of the embodiments described herein, the polymer layer can be formed by surface-initiated ATRP (SI-ATRP) of oligo(ethylene glycol)methyl methacrylate (OEGMA) to form a poly(OEGMA) (POEGMA) film. In an embodiment, the polymer layer is a functionalized POEGMA film prepared by copolymerization of a methacrylate and methoxy terminated OEGMA. Suitably, the POEGMA polymer can be formed in a single step.

In general, the brush molecules formed by the processes described herein (or other processes either known in the art or which will be apparent to those skilled in the art), can be from 2 or 5 up to 100 or 200 nanometers in length, or more, and can be deposited on the surface portion at a density of from 10, 20 or 40 to up to 100, 200 or 500 milligrams per meter, or more.

Protein resistant groups can be hydrophilic head groups or kosmotropes. Examples can include but are not limited to oligosaccharides, tri(propyl sulfoxide), hydroxyl, glycerol, phosphorylcholine, tri(sarcosine) (Sarc), N-acetylpiperazine, betaine, carboxybetaine, sulfobetaine, permethylated sorbitol, hexamethylphosphoramide, an intramolecular zwitterion (for example, —CH₂N⁺(CH₃)₂CH₂CiI₂CH₂SO₃″) (ZW), and mannitol.

Additional examples of kosmotrope protein resistant head groups can include, but are not limited to:
—(OCH₂CH₂)₆OH;
—O(Mannitol);
—C(O)N(CH₃)CH₂(CH(OCH₃))4CH₂OCH₃;
—N(CH₃)₃⁺C17-SO₃″Na⁺(1:1);
—N(CH₃)₂⁺CH₂CH₂SO₃
—C(O)Pip(NAc) (Pip=piperazinyl)
—N(CH₃)₂⁺CH₂CO₂″;
—O([Glc-a(1,4)-Glc-P(1)1); —C(O)(N(CH₃)CH₂C(O))3N(CH₃)2;
—N(CH₃)₂⁺CH₂CH₂CH₂SO₃″;
—C(O)N(CH₃)CH₂CH₂N(CH₃)P(O)(N(CH₃)₂)₂ and
—(S(O)CH₂CH₂CH₂)₃S(O)CH₃.

In some of the embodiments described herein, a suitable protein resistant head group can comprise poly(ethylene glycol) (PEG), for example PEG of from 3 to 20 monomelic units.

Prior to deposition of further components onto the polymer layer, the substrate with the optional linking layer and polymer layer can be dry or at least macroscopically dry (that is, dry to the touch or dry to visual inspection, but retaining bound water or water of hydration in the polymer layer). For example, to enhance immobilization of a capture agent, the polymer layer can suitably retain bound water or water of hydration, but not bulk surface water. If the substrate with the linking layer and polymer layer has been stored in desiccated form, bound water or water of hydration can be reintroduced by quickly exposing the polymer layer to water (e.g., by dipping in to water) and subsequently blow-drying the surface (e.g., with a nitrogen or argon jet). Alternatively, bound water or water of hydration can be reintroduced by exposing the polymer layer to ambient air for a time sufficient for atmospheric water to bind to the polymer layer.

### VII. Capture Region

The device comprises at least one capture region comprising at least one capture agent, which can be non-covalently bound to the polymer layer. The number of capture regions can vary widely and can depend on several factors including the size and shape of the substrate, the intended use of the device (e.g., a point-of-care diagnostic, a panel array (e.g., microarrays for screening DNA, MM Chips (microRNAs), protein, tissue, cellular, chemical compounds, antibody, carbohydrate, etc.), and the like. The capture agent comprising a capture region is generally one member of a specific binding pair. Examples of suitable capture agents can include, but are not limited to, antigens, antibodies, peptides, proteins, nucleic acids, nucleic acid or peptide aptamers, ligands, receptors, and the like. Embodiments relate to a device comprising a plurality of capture regions that can comprise a plurality of different capture agents such as a diagnostic panel array.

In some embodiments, the capture agent can comprise a biomarker associated with any disease, disorder, or biological state of interest. Accordingly, the selection of the capture agent can be driven by the intended use or application of the device and methods described herein and can include any molecule known to be associated with a disease, disorder, or biological state of interest, or any molecule suspected of being associated with a disease, disorder, or biological state of interest. Thus, the selection of a capture agent is within the ability of one skilled in the art, based on the available knowledge in the art.

In some of the embodiments described herein, the capture agent can comprise a biomarker associated with any microbial infection of interest, examples of which can include but are not limited to: Anthrax, Avian influenza, Botulism, Buffalopox, Chikungunya, Cholera, Coccidioidomycosis, Creutzfeldt-Jakob disease, Crimean-Congo haemorrhagic fever, Dengue fever, Dengue haemorrhagic fever, Diphtheria, Ebola haemorrhagic fever, Ehec (E. coli 0157), Encephalitis, Saint-Louis, Enterohaemorrhagic Escherichia coli infection Enterovirus, Foodborne disease, Haemorrhagic fever with renal syndrome, Hantavirus pulmonary syndrome, Hepatitis, Human Immunodeficiency Virus (HIV), Influenza, Japanese encephalitis, Lassa fever, Legionellosis, Leishmaniasis, Leptospirosis, Listeriosis, Louseborne typhus, Malaria, Marburg haemorrhagic fever, Measles, Meningococcal disease, Monkeypox, Myocarditis Nipah virus, O'Nyong-Nyong fever, Pertussis, Plague, Poliomyelitis, Rabies, Relapsing fever, Rift Valley fever, Severe acute respiratory syndrome (SARS), Shigellosis, Smallpox vaccine—accidental exposure, Staphylococcal food intoxication, Syphilis, Tularaemia, Typhoid fever, West Nile virus, Yellow fever, etc.

The capture agent can be deposited on the polymer layer by any suitable technique such as microprinting or microstamping, including piezoelectric or other forms of non-contact printing and direct contact quill printing. When the capture agent is printed on to the polymer layer, it can suitably be absorbed into the polymer layer such that it remains bound when the device is exposed to a fluid, such as a biological fluid. The brush polymer can also provide a protective environment, such that the capture agent remains stable when the device is stored. For example, in embodiments in which the capture agent is a peptide or protein, such as an antigenic protein or an antibody, a brush polymer layer can protect the capture agent against degradation, allowing the device to be stored under ambient conditions.

When an array is formed by the deposition of multiple capture agents at discrete locations on the polymer layer, probe densities of 1, 3, 5, 10, 100 or up to 1000 probe locations per cm² can be made. Modern non-contact arrayers can be used in the deposition step to produce arrays having up to 1,000,000 probe locations per cm². For example, using dip-pen nanolithography, arrays with up to 1 billion discrete probe locations per cm² can be prepared. It will be appreciated that the specific molecular species at each capture spot can be different, or some can be the same (e.g., to provide some redundancy or control), depending upon the particular application, as described herein.

The capture agent can be printed onto the polymer layer to form the capture region. The capture region(s) can be arranged in any particular manner and can comprise any desirable shape or pattern such as spots (e.g., of any general geometric shape), lines, or other suitable patterns that allow for identification of the capture region on the surface of the polymer and substrate. In embodiments, a plurality of capture agents can be arranged in a predetermined pattern such that the identity of the capture agent is associated with a specific location on the substrate.

### VIII. Detection Agent

The embodiments of the present disclosure may optionally comprise at least one detection agent and/or an excipient. In some of the embodiments described herein, a capture agent can remain non-covalently bound to the polymer layer (e.g., polymer brush) upon contact with a fluid such as a biological fluid, buffer, or aqueous solvent, while the excipient present in the labile region can absorb in to the polymer brush and block absorption of the detection agent. Accordingly, when exposed to an aqueous fluid such as a sample comprising a biological fluid, the detection agent can be solubilized and release in to the fluid, and can bind to an analyte of interest. The excipient can also further stabilize the detection agent during storage.

In some of the embodiments described herein, the detection agent can comprise a compound capable of binding to a second member of a specific binding pair. When solubilized and released in to the sample (e.g., a biological fluid), if the second member of the specific binding pair is present in the fluid, it can bind to the detection agent. The second member can then bind to the capture agent in the capture region of the device. Alternatively, the detection agent can encounter the second member of a specific binding pair when already bound to the capture agent. For example, if the capture agent is an antigenic protein and the analyte is a patient-generated antibody that can specifically bind the antigenic protein, the detection agent can comprise an anti-human antibody.

In some of the embodiments described herein, the labile region can comprise two different agents to form a “sandwich” type assay. In such embodiments, a first agent can bind to the analyte while the other agent binds to the first agent to form a “sandwich” which can then bind to the capture agent. For example, the detection agent can comprise biotin, which can bind avidin or streptavidin that is functionalized with a detection moiety.

The detection agent further comprises a detectable moiety that, directly or indirectly, provides a detectable signal. Exemplary detection moieties can include, but are not limited to, fluorophores, chromophores, radiolabels, polynucleotides, small molecules, enzymes, nanoparticles, and upconverters. In some of the embodiments described herein, the detection moiety can be a fluorophore such as a cyanine (e.g., CyDyes such as Cy3 or Cy5), a fluorescein, a rhodamine, a coumarin, a fluorescent protein or functional fragment thereof, or it can comprise a small molecule such as biotin, or it can comprise gold, silver, or latex particles.

In some of the embodiments described herein, the excipient can be a molecule or a combination of molecules that is selected as to allow for a stable, but non-permanent, association between the detection agent and the polymer. In embodiments the excipient can be partially soluble, substantially soluble or soluble in an aqueous solution (e.g., buffer, water, sample, biological fluid, etc.). In such embodiments, the excipient can be selected from the non-limiting examples of salts, carbohydrates (e.g., sugars, such as glucose, fucose, fructose, maltose and trehalose), polyols (e.g., mannitol, glycerol, ethylene glycol), emulsifiers, water-soluble polymers, and any combination thereof. Such excipients are well known in the art and can be selected based on the interaction between the excipient and detection agent, the excipient and the polymer, the solubility of the excipient in a particular medium, and any combination of such factors. In some of the embodiments described herein, the excipient can comprise PEG.

The detection agent and the excipient can be deposited on the polymer layer by any suitable technique such as microprinting or microstamping, including piezoelectric or other forms of non-contact printing and direct contact quill printing. A mixture of the detection agent and the excipient can be deposited simultaneously, or the excipient can be deposited prior to the detection agent.

When an array is formed by the deposition of multiple detection agents at discrete locations on the polymer layer, probe densities of 1, 3, 5, 10, 100 or up to 1000 probe locations per cm² can be made. Modern non-contact arrayers can be used in the deposition step to produce arrays having up to 1,000,000 probe locations per cm². For example, using dip-pen nanolithography, arrays with up to 1 billion discrete probe locations per cm² can be prepared. It will be appreciated that the specific molecular species at each capture spot can be different, or some can be the same (e.g., to provide some redundancy or control), depending upon the particular application.

### IX. Other Elements

Some of the embodiments described herein can further comprise an agent to demarcate a patterned region on the polymer layer, such that a fluid (e.g., a biological fluid) will remain confined to a specified region on the polymer layer such that it contacts the capture region and the labile region. Such an agent can be, for example, a hydrophobic ink printed on the polymer layer prior to the deposition of the capture agent and the components of the labile region. Alternatively, the agent can be a wax. In other embodiments, the sample can be contained or directed on the device through selection of an appropriate geometry and/or architecture for the substrate, for example, a geometry that allows the sample to diffuse to the regions comprising the capture agent and the components of the labile spot. In some of the embodiments described herein, the substrate can comprise a well, or a series of interconnected wells.

Some of the embodiments described herein can comprise an anticoagulant to prevent the blood from clotting. Exemplary anticoagulants can include but are not limited to vitamin K antagonists such as Coumadin, heparins, and low molecular weight heparins.

Some of the embodiments described herein can further comprise regions printed with control agents. For example, when the detection agent comprises an anti-human antibody, control capture regions of human IgG can be printed alongside the capture regions to verify the activity of the anti-human detection antibody and to normalize the signal from the detection moiety, such as fluorescence intensities.

### X. Device Storage

After deposition of the capture agent, detection agent, excipient and other optional components, the device is optionally dried, e.g., by mild desiccation, blow drying, lyophilization, or exposure to ambient air at ambient temperature, for a time sufficient for the article to be dry or at least macroscopically dry as described above. Once the device is dry or at least macroscopically dry, it can be sealed in a container (e.g., such as an impermeable or semipermeable polymeric container) in which it can be stored and shipped to a user. Once sealed in a container, the device can have, In some of the embodiments described herein, a shelf life of at least 2 to 4 months, or up to 6 months or more, when stored at a temperature of 25° C. (e.g., without loss of more than 20, 30 or 50 percent of binding activity). In some of the embodiments described herein, a shelf life of at least 2 to 4 months, or up to 6 months or more, when stored at a temperature of 2-8° C. (e.g., without loss of more than 20, 30 or 50 percent of binding activity). In some of the embodiments described herein, a shelf life of at least 2 to 4 months, or up to 6 months or more, when stored at a temperature of −20° C. (e.g., without loss of more than 20, 30 or 50 percent of binding activity).

### XI. Detection

Following exposure of a device described herein to a biological sample (e.g., a biological fluid), a signal from the detection agent can be detected using any suitable method known in the art. Exemplary methods can include, but are not limited to, visual detection, fluorescence detection (e.g., fluorescence microscopy), scintillation counting, surface plasmon resonance, ellipsometry, atomic force microscopy, surface acoustic wave device detection, autoradiography, and chemiluminescence. As one of skill in the art will appreciate, the choice of detection method will depend on the specific detection agent employed.

### XII. Detector

In some embodiments, the present disclosure provides a detector for use in the methods of the disclosure. A detector is typically configured to hold a chip and is equipped with one or more light sources. The light sources are configured to illuminate the chip. The light soured may illuminate the chip such that light passes through the chip. The light source may illuminate the chip such that light contacts a surface of the chip at an angle from about 0 degrees to about 90 degrees. In one embodiment, a light source may be positioned such that the light illuminates one surface of the chip and a non-fouling polymer layer disposed in a channel on the other side of the surface. In embodiments of this type the light may pass through on surface of the chip but not through the entire chip.

The light sources may be of any type, for example, may be LED lights. One suitable example of a light source for use in the present disclosure is a LED370F produced by Thor Laboratories. In some embodiments, a detector may comprise more than one light source, for example, may comprise 2, 3, 4, 5, 6, 7, 8, 9, 10, or more light sources. The light sources may be the same or different. Each light source may produce light of the same or different wavelength as light produced by another light source. Each light source may produce light of the same power. In one embodiment, 4 light sources are used each with a forward optical power of 2 mW. A detector of the disclosure will also typically comprise a lens. The lens is configured to collect light, e.g. light emitted by a fluorophone, and direct it to a camera of a smart phone.

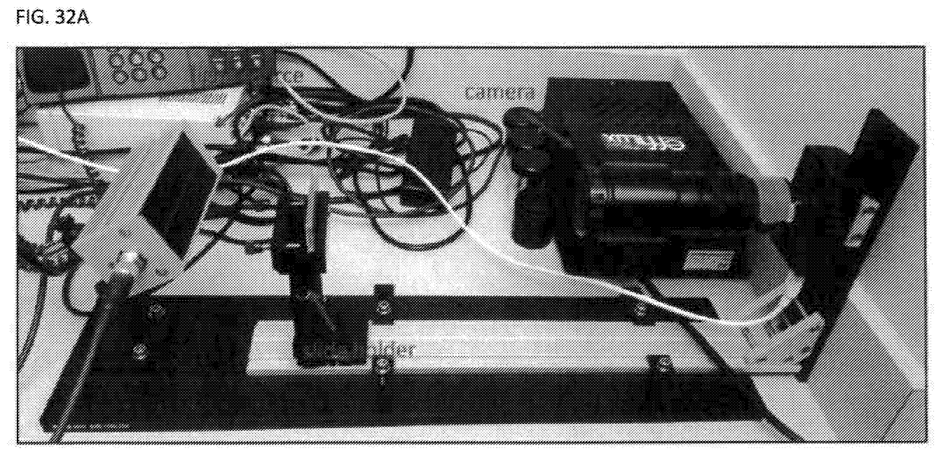

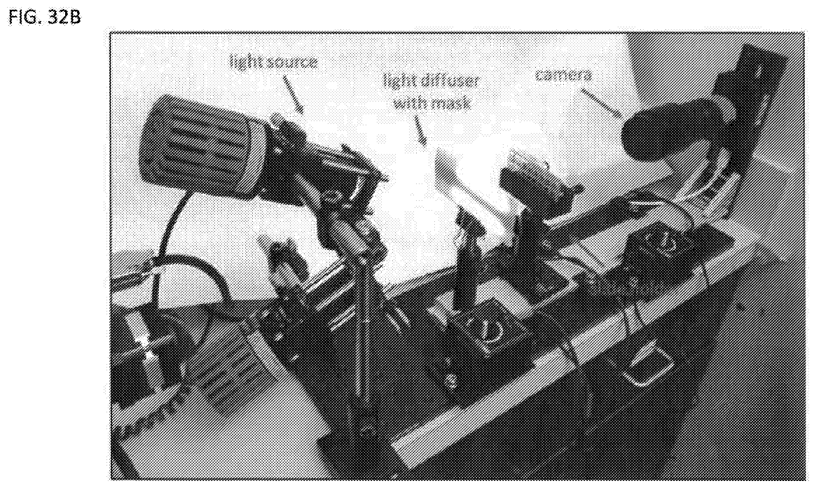

One exemplary detector is provided in FIG. 32A. In one embodiment, the detector comprises a camera, a light source, and a slide holder. In one embodiment, the light source is directed through a slit towards the slide. In one embodiment, the slide is held in an upright position. In one embodiment, a camera is positioned on the other side of the slide and captures the scattering exhibited by the cells bound to the array. Another exemplary detector is provided in FIG. 32B. In one embodiment, two beams are used in the detector. In one embodiment, the light beams are directed through slits toward the slide. In one embodiment, the slide is held in an upright position. In one embodiment, a diffuser with a mask is placed between the light source and the slide. In one embodiment, a camera is positioned on the other side of the slide and captures the scattering exhibited by the cells bound to the array. In one embodiment, depending on the light source, a diffuser with a mask can also be used.

A detector of the disclosure may comprise a magnetic portion designed to magnetically attach the detector to a cell phone.

### XIII. Kits

Inventive embodiments herein can include kits. Kits can comprise the supports, solid supports, and medical devices herein. Kits can include instructions, for example written instructions, on how to use the material(s) therein. Material(s) can be, for example, any substance, composition, polynucleotide, solution, etc, herein or in any patent, patent application publication, reference, or article that is incorporated by reference.

A kit can include a composition or a device as described herein, and optionally additional components such as buffers, reagents, and instructions for carrying out the methods described herein. The choice of buffers and reagents will depend on the particular application, e.g., setting of the assay (point-of-care, research, clinical), analyte(s) to be assayed, the detection moiety used, etc. For example, if the capture agent is a polynucleotide and the analyte of interest is a complementary polynucleotide, the kit can include a lysis buffer to be added to the sample of biological fluid, to make the polynucleotide from the sample available for binding.

The kit can also include informational material, which can be descriptive, instructional, marketing or other material that relates to the methods described herein and/or the use of the devices for the methods described herein. In embodiments, the informational material can include information about production of the device, physical properties of the device, date of expiration, batch or production site information, and so forth.

### XIV. Assays

In certain embodiments provided by the present disclosure, testing for anti-A and anti-B is done in the same well, requiring only one well per sample. In another embodiment, both the IgM and IgG forms of these antibodies are detected, as opposed to IgM only (or mostly IgM, which is more likely to cause the agglutination than IgG), which could result in better sensitivity, especially for those donors that have very low anti-A or anti-B that is IgM in nature.

In one embodiment, the assay detects expected antibodies anti-A and anti-B from donor plasma in a microarray format on a POEGMA or epoxy POEGMA substrate using a printed array of antigens A and B as the capture reagents and antigen positive red blood cells as the detection reagents. Once the reaction has taken place, the slide is turned on its side and unbound cells are removed from the printed spots by gravity, while cells bound to spots containing captured anti-A or anti-B remain bound. Due to the specific nature of the interactions between the antibodies and both the capture and detection reagents, detection of non-specific binding to the negative spots, as well as the background, are almost entirely eliminated.

In another embodiment, the format of the present disclosure can also be used for ABO forward typing, in which the printed array contains Anti-A and Anti-B as the capture reagents and the donor red blood cells are introduced to the array as the sample. After the reaction takes place, the slide is turned on its side and unbound cells are removed from the printed spots by gravity, while cells bound to printed spots containing anti-A or anti-B reagents remain bound. In one embodiment, in the microarray format, 16 samples can be tested on one slide for either forward or reverse typing. In another embodiment, 8 samples can be tested on one slide for both forward and reverse typing.

In one embodiment, the slide is imaged with the slide still turned on its side with a scattering imaging system. In one embodiment, the imaging system consists of a light source directed at the slide held in place by a holder, with a camera on the opposite side of the slide to capture the scattering from the bound cells. In one embodiment, the light source consists of either one or two beams directed through a slit. In one embodiment, depending on the light source, a diffuser with a mask can also be used.

In one embodiment, the detection reagent consists of antigen positive cells with a naturally occurring confirmation of surface antigens that can detect both the IgM and IgG forms of anti-A and anti-B, making the technique sensitive. In another embodiment, the method is tested with adapters that form wells with volumes as low as 40 pico liter (pl), a reduced volume compared to testing in tubes or wells.

In comparison to other microarray immunoassay methods, for reverse typing, both the capture and detection steps are specific in nature, almost entirely eliminating detection of any nonspecific or undesired binding to the negative spots or the substrate background.

Imaging the microarray flat would introduce complications such as re-dispersion of the unbound detection cells, but this scattering imaging system allows for analysis of the results while the slide is still upright.

In one embodiment, the present disclosure provides an ABO Reverse Typing Assay. In one embodiment, antigens A and B are printed in a 16-field microarray format on a POEGMA or epoxy POEGMA substrate. In one embodiment, the slide is fitted with a 16-well adapter. In one embodiment, the wells are exposed to donor plasma, then washed. In one embodiment, the antigen positive detection cells are added to the wells. In one embodiment, the wells are sealed with transparent tape, and the slide is turned on its side to allow the unbound detection cells to settle to the bottom of the wells.

In another embodiment, the present disclosure provides an ABO Forward Typing Assay. In one embodiment, Anti-A and Anti-B antibodies are printed in a 16-field microarray format on a POEGMA or epoxy POEGMA substrate. In one embodiment, the slide is fitted with a 16-well adapter. In one embodiment, the wells are exposed to donor red blood cells. In one embodiment, the wells are sealed with transparent tape, and the slide is turned on its side to allow the unbound cells to settle to the bottom of the wells.

In another embodiment, the present disclosure provides an imaging system. In one embodiment, the slide is held in place in the same orientation with the slide holder. In one embodiment, the light source is aimed at the middle of the slide between the wells (if one light beam) or at the top and bottom of the slide above and below the wells (if two light beams). In one embodiment, a camera on the other side of the slide captures the scattering image.

In one embodiment, the light scattering image collected by the camera captures not only the scattering from the cells that are bound to the array, but also scattering from the solution contained in the wells, scattering from any bubbles in the wells, scattering from the sealing tape, and scattering from the substrate. This limitation can be overcome by careful omission of bubbles during sealing, avoidance of smudges and scratches on the tape and substrate, and/or using an adapter with ports that can be sealed with a sealant such as clear nail polish instead of tape. Extraneous scattering can also be reduced by adjusting the parameters of the imaging system.

In a study in which 53 plasma donors were tested (15 Type A, 15 Type B, 8 Type AB, and 15 Type O), a best point estimate of 96.6% was achieved for both anti-A and anti-B in donor plasma. The study included many samples known to be challenging due to the low titer anti-A or anti-B present in the sample, so the agreement is expected to increase with the number of samples.

In one embodiment, the assay and imaging system can be incorporated into a blood typing instrument. The microarray-based assay is specific and sensitive, and can type 16 plasma donors on a single slide, with the possibility of increasing this number by using an adapter that forms 24 or 32 wells. Analysis of the results of the assay by scattering imaging would be more cost effective than the lasers required to analyze fluorescence-based microarray assays.

ABO blood group testing descriptions can be found in various sources, such as The Blood Bank and the Technique and Therapeutics of Transfusions, by Robert A. Kilduffe. St. Louis, The C.V. Mosby Company, 1942.

In one embodiment, the scattering imaging system was developed to replicate the type of image captured with a cell phone camera while holding the slide up to the light, but with the following enhancements: the slide would be in a fixed position, better resolution and sensitivity, and ability to easily control parameters such as the angle and intensity of the light.

In other embodiments, assays detecting multiple human antibodies (i.e. IgG, IgM) (i.e. directed against red blood cell epitopes, HLA, Infectious Disease agents) simultaneously in a specific microarray design using a “double antigen sandwich format” (antigen bridge assay) are also provided.

In some examples, multiple antibodies are detected simultaneously and individually within a single incubation step of the assay components and the specimen.

In some examples, the antigen bridge assay is particularly adapted/suited for blood donor screening, e.g., by identifying multiple antibodies (against red cell antigens, HLA, infectious agents). The assay format is fast, highly sensitive, and provides superior specificity when compared with traditional antibody detection assays using secondary anti-human IgG and/or IgM.

Assays for the detection of human antibodies are known. These assays typically detect antibodies directed against a single infectious agent at a time, such as antibodies against HIV, HBV, HCV, HTLV, TP, and CMV. Screening in a blood donor setting requires multiple tests involving anti-human IgG or anti-human IgM antibodies. The current state of the art tests for these antibodies in a variety of assays on multiple instrument systems with different assay formats (partly anti-human IgG/IgM, partly double antigen sandwich).

In addition, known assay formats may detect only one subset of antibodies (IgG or IgM) at a time. However, a sensitive and comprehensive detection of infectious disease antibodies requires the detection of both human IgG and IgM. The double antigen sandwich format is highly specific and avoids non-specific reactivity typically seen with anti-human IgG/IgM formats. The high specificity of the double antigen format overcomes current limitations of assay specificity, such as false positive reactions for TP, CMV, HBV core antibodies, HCV, and the like.

The described format allows the simultaneous and individual detection of multiple antibodies with a single incubation in one reaction (one step design or one-step incubation).

The described assay format significantly increases the efficiency and reduces the time to report a diagnosis to a donor/patient by avoiding reflexive, sequential testing procedures. Avoids or reduces greatly the need for reflex testing, where initially reactive samples are re-tested to confirm reactivity.

Multiple antibodies, directed against human epitopes (Red cell, HLA, platelets) and infectious agents impacting the decision of suitability for transfusion can be specifically detected in one reaction and in parallel.

The exhaustive detection of antibodies could provide greater sensitivity to the assays and allow earlier diagnosis of an infection. The sensitive detection of antibodies to infectious disease agents and the specific detection of other human antibodies is crucial in blood donors to allow a safe transfusion of collected blood and components. The screening of the absence for these antibodies also has to be highly specific, since donors tested positive may be dismissed from further donations.

In one embodiment, the design provided by the present disclosure combines microarray technology with a reaction and detection format. In some examples, the technology involves the creation of a tertiary complex of an antigen bound to a solid surface (e.g., a non-fouling surface), a liquid phase labeled antigen and human immunoglobulin specifically binding to at least one solid phase antigen and one labelled antigen. The design allows specifically the simultaneous detection of antibodies against different entities like infectious disease agents, red cell antigens, platelet antigens, HLA antibodies, etc.

An exemplary assay involving the detection of antibodies; e.g., antibodies to TP is shown in Example 13.

In some examples according to this embodiment, the assay is a one-step-assay, in which the bound (solid phase) antigen, the liquid phase labeled antigen, and the specimen containing the antibodies are incubated at the same time in one reaction. In an array format, after one separation step, all antibodies can be detected on the different areas/spots of the microarray respectively.

In other examples, the assay format is a two-step assay, where in a first step bound antigen, a soluble antigen with a generic label (e.g., biotin), and the sample are incubated to form a tertiary complex (e.g., bound antigen-antibody from sample-labeled soluble antigen). After a washing step, streptavidin with any kind of detection label (e.g., enzymatic or fluorescent) is attached to the complex and subsequently detected.

In some embodiments labeled antigens and non-labeled antigens are deposited onto the solid surface. In some examples, the non-labelled antigen is bound covalently and/or through adsorption to the surface and will remain bound, even when incubated with the biological sample (e.g., containing at least one antibody). The labeled antigen is releasably bound to the surface and is released from the surface upon incubation with the sample containing the antibodies. In this design, upon incubation with the sample, tertiary complexes can form in the reaction. This design is particularly suitable for point of care applications, where a minimum of reagents (ideally only the biological sample) is added to the microarray.

In some embodiments, the capture antigen is bound to a substrate containing a POEGMA coating. In some examples, the POEGMA coating is an epoxy-POEGMA coating, or another modified POEGMA, which allows for the covalent attachment of the capture antigen to the POEGMA surface.

### EXAMPLES

The following non-limiting Examples are intended to be purely illustrative, and show specific experiments that were carried out in accordance with the disclosure.

### Example 1: Synthesis of e-POEGMA

POEGMA coating technology of the present disclosure was modified by adding reactive functional groups (e.g., epoxide groups) to the POEGMA composition during the polymerization process. The functional groups provide covalent chemical bonding for the immobilization of biomolecules without compromising the non-fouling characteristics of the POEGMA surface. Epoxy-co-POEGMA (e-POEGMA, or “e-PO”) was synthesized by adopting procedures known for the synthesis of non-functionalized POEGMA.

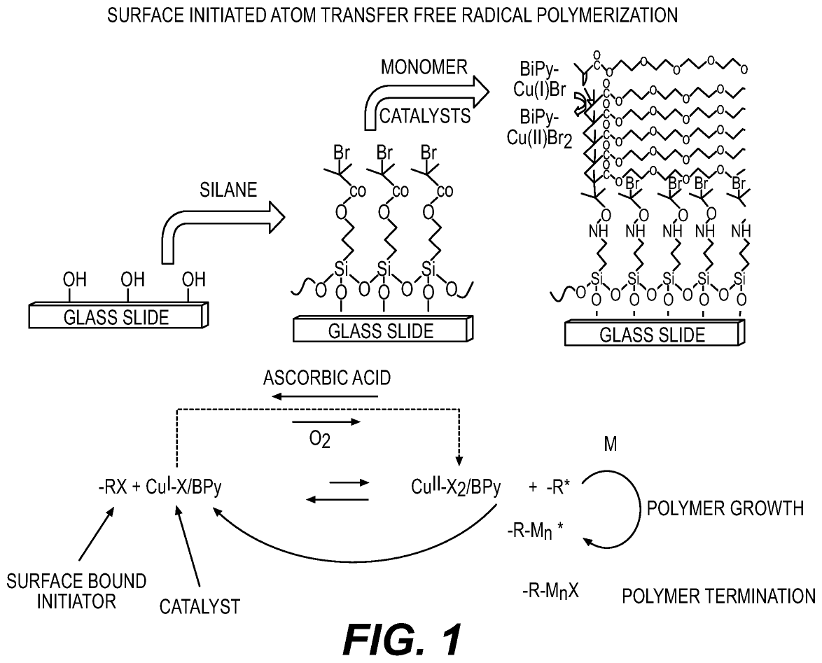

POEGMA synthesis includes two main steps: (1) initiator coating and (2) polymer growth. The polymer growth step determines the chemical content of the POEGMA coating. As shown in FIG. 1, step 1 coats the substrate with an initiator, e.g., 3-(trimethoxysilyl) propyl-2-bromo-2-methylpropionate, e.g., via molecular vapor deposition. In step 2, POEGMA is grown on the substrate by surface initiated atom transfer radical polymerization (SI-ATRP). Step 1 can be done using commercial equipment MVD300 (Applied Microstructure, San Jose, Calif.). Step 2 can be performed in an oxygen free environment (e.g., in a glovebox).

I. Traditional POEGMA: Non-Reactive Surface

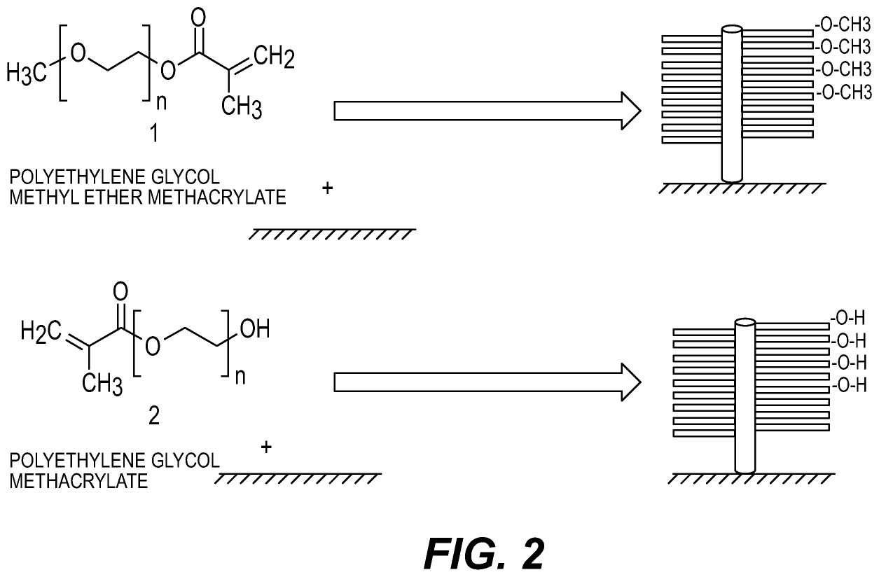

Traditional POEGMA is synthesized from PEG-based monomers without reactive functional groups that would provide chemical binding capabilities. Exemplary POEGMA substrates include those made from poly(ethylene glycol) methyl ether methacrylate (PEGMEM, e.g., with an average molecular weight of about 300) and poly(ethylene glycol) methacrylate (PEGMA, e.g., with an average molecular weight of about 360) (FIG. 2). PEGMEM provides methyl ether (—OCH3) terminated POEGMA side chains (brushes) and PEGMA provides hydroxyl terminated POEGMA side chains. These two types of POEGMA behaved similarly as non-fouling surfaces that also provide passive physical adsorption when biomolecules were deposited onto them.

II. Reactive Surface: Epoxy-Co-POEGMA (e-POEGMA)

In microarray printing, biomolecular probes are immobilized on substrates and secured via various mechanisms. If physical adsorption is not sufficient to anchor the probes, covalent chemical bonds provide stronger binding. A number of reactive chemical groups can be used for immobilizing a biomolecule to a polymer surface. An exemplary reactive group is an epoxide group, which can react with a variety of functional groups from biomolecules, such as amines, thiol groups, and carboxyl groups. In addition, the reactions with epoxide groups are spontaneous but slow at ambient temperature.

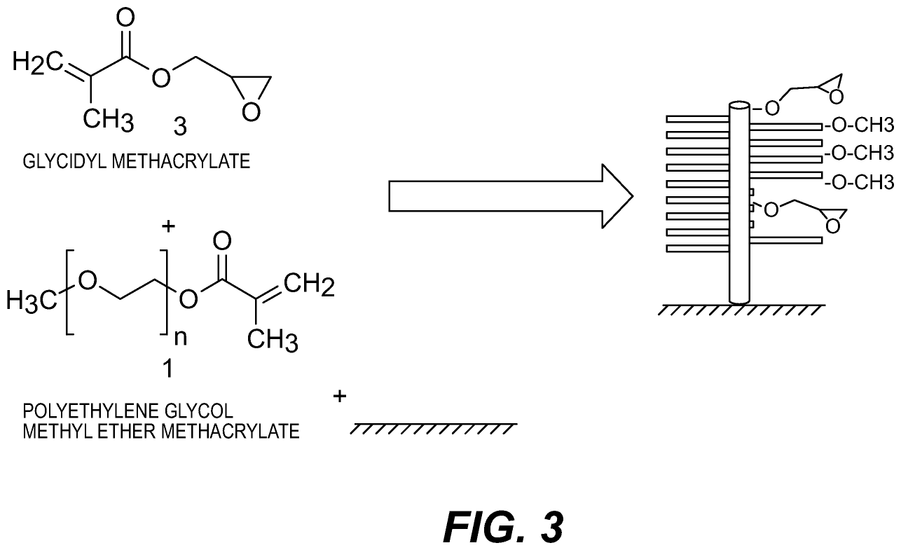

The main difference between POEGMA and e-POEGMA synthesis is the starting monomers. The regular POEGMA synthesis starts from pure PEG based monomers such as PEGMEM or PEGMA. Since both PEGMEM and PEGMA are reasonably soluble in water, pure water can be used as solvent. An exemplary e-POEGMA was synthesized from a mixture of PEGMEM and glycidyl methacrylate (GMA, MW 142) (FIG. 3). In the copolymer, PEGMEM contributes non-fouling properties while GMA contributes chemical bonding capabilities. Since GMA is not soluble in pure water, certain amount of ethanol is added to form homogeneous mixture. Typically, 10-20% ethanol is sufficient, depending on the concentrations of monomers in the polymerization mixture.

III. Effect of GMA to PEGMEM Ratio

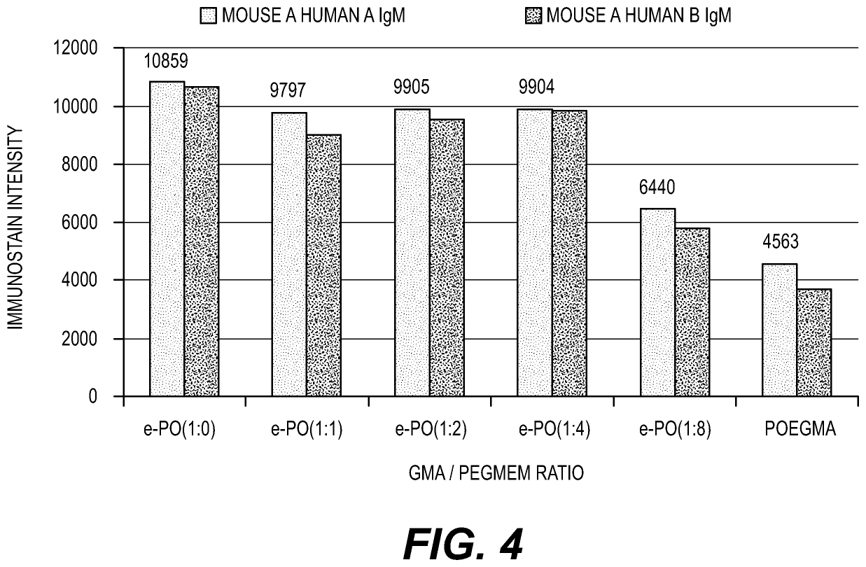

e-POEGMA surfaces were synthesized using GMA/PEGMEM volume ratios of 1:0, 1:1, 1:2, 1:4 v/v, and 1:8 v/v on glass slides. Immunostaining assays were performed on these slides following the procedure described in Example 4 (Antibody Staining Assay). Intensities measured in the immune-staining assay are summarized in FIG. 4. An increase in assay intensities was observed for substrates with GMA/PEGMEM ratios of 1:8 to 1:4. Interestingly, a further increase of the GMA/PEGMEM ratio (e.g., 1:2) did not have a notable effect on assay intensities. There is 2-fold intensity increase from regular POEGMA to e-POEGMA with a GMA/PEGMEM ratio of 1:4 v/v. When a GMA/PEGMEM ratio is above 1:4, the reaction solution became cloudy and the resulting e-POEGMA coating exhibited increased roughness. The e-PEOGMA prepared using a GMA/PEGMEM ratio of 1:4, is referred to herein as e-PO4. The growth rate for e-PO4 is 2-4 times faster than for POEGMA.

### Example 2: IL-6 Immunoassay on e-POEGMA

In protein microarray immunoassay, detection sensitivity is one of the most important criteria to judge different systems. To compare POEGMA with e-POEGMA, a model immunoassay that detects human interleukin-6 (IL-6) was implemented. For immunoassays, such as IL-6 cytokine detection assays and infectious disease screening assays, microarrays based on e-POEGMA exhibited significantly higher detection sensitivities than those based on traditional POEGMA. IL-6 antibodies and antigens are commercially available. An exemplary assay procedure is described herein below.

IL-6 antigen detection were studied under (1) model conditions (PBS) and real life conditions (human serum or plasma). In the model conditions, samples were prepared by spiking IL-6 antigen into PBS containing 10% fetal bovine serum (FBS). Samples were placed onto the microarray surface and incubated for 4 hours for more complete binding. In the assays resembling real life conditions, samples were prepared by spiking IL-6 into human plasmas or sera from individual donors. Samples were placed onto the microarray surface and incubated for 1 hour to simulate typical immunoassay conditions.

I. Model Assay Conditions—PBS Solution

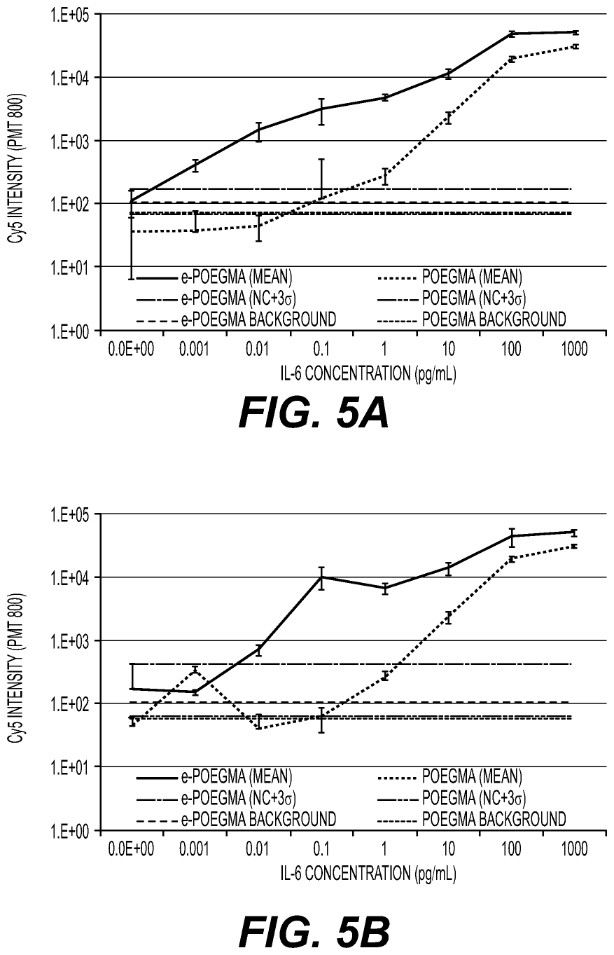

FIG. 5A and FIG. 5B show fluorescence intensities (Cy5 probe) and corresponding background intensities from the IL-6 antigen titrations, which were performed in duplicates. The mean intensity from the blank plus 3 standard deviation is used to determine the limit of detection for each titration assay. The detection limit measured for e-PO4 was about 10⁻² pg/mL. The detection limit measured for POEGMA was about 1 pg/mL. Although there was some variation between the two experiments, assay sensitivity was about 1-2 orders of magnitude higher on e-PO4 than on POEGMA. The background intensities from both substrates were comparable.

II. Assays in Human Serum/Plasma

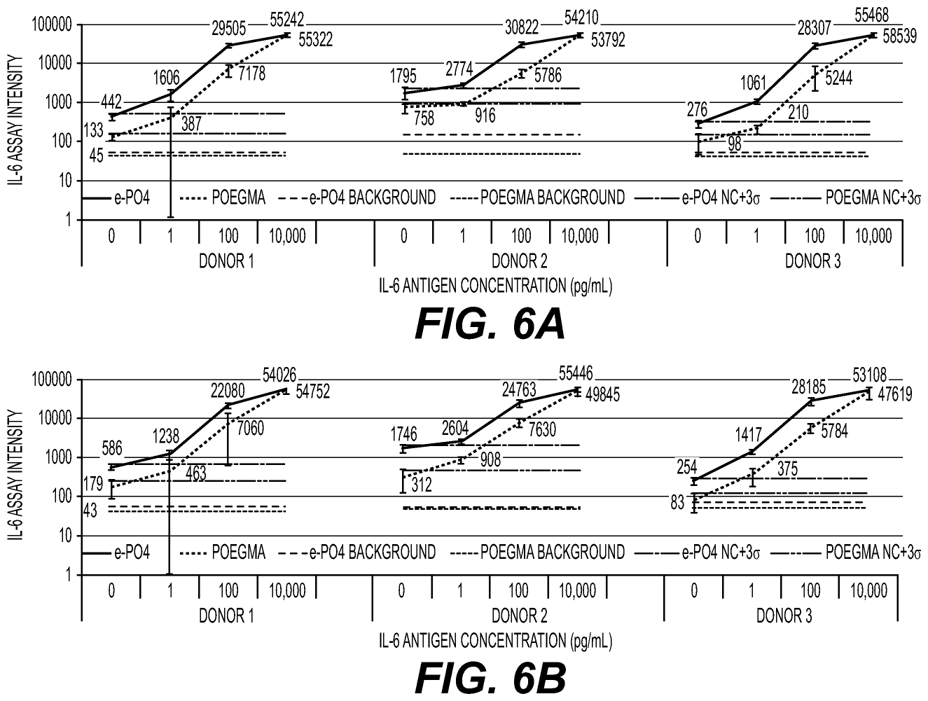

IL-6 assay sensitivities in plasma and serum were lower than those found for the above model assay conditions. Results are summarized in FIG. 6A and FIG. 6B. On e-PO4, the IL-6 detection limit was about 1 pg/mL. On POEGMA, the IL-6 detection limit was about 100 pg/mL. It is notable that the background intensities for the IL-6 assay in plasma/serum was comparable to those measured in PBS on both POEGMA and e-PO4 substrates.

The following is an exemplary experimental procedure for the immunoassay procedure.

### (a) Anti-Human IL-6 Microarray Printing

Anti-human IL-6 IgG antibody (R&D System, Minneapolis, Catalog No: MAB206) was printed on the substrates (e.g. POEGMA-coated glass slide), using SCIENION noncontact piezo-dispenser sciFLEXARRAYER S3 (SCIENION AG, Berlin, Germany). The printing solution contains 0.5 mg/mL IL-6 antibody in 1×PBS with 0.01% (v/v) Tween-20.

### (b) Post-Print Incubation

The protein microarrays were incubated on printer stage for 1 hour at a relative humidity of 50-55%, immediately following printing. Subsequently, the microarray slides were incubated overnight at 40° C. in a slide dryer, SHURDry™ Slide Dryer II (General Data Healthcare, Fisher Scientific) to facilitate immobilization of the printed antibodies. The microarray slides were stored in sealed pouch with desiccant at 2-8° C.

### (c) Pre-Assay Wash

Prior to assay, the microarray slides were mounted on the ProPlate® Multi-Array Slide System (16-well module, Grace Bio Labs, Oregon) and washed by using BioTek Strip Washer Elx50 8-Channel Manifold (BioTek, Vermont) with PBS containing 0.1% tween-20. The slides were used immediately after washing without drying in-between.

### (d) IL-6 Antigen Incubation

Recombinant human IL-6 antigen (R&D System, Minneapolis, Catalog No-206-IL) was prepared in desired buffer and added to the arrays on the slides, and then incubated at room temperature on an orbital shaker (100 RPM) for specified period of time (typically 1-4 hours).

### (e) Detection Antibody Incubation

After incubation, the antigen solution was aspirated. Immediately after aspiration, the microarrays were incubated with 2004, of biotinylated human IL-6 antibody (R&D System, Minneapolis, Catalog No: BAF206) at RT for 1 hour on an orbital shaker at 100 RPM. After incubation, the antibody solution was aspirated, followed by staining with 200 μL of 5 mg/mL Cy5-labeled Streptavidin (Jackson Immuno Research Laboratories, PA; Catalog No-016-170-084) in 1×PBS with 1% BSA for 30 min. at RT.

### (f) Post-Assay Wash

After immunostaining, microarrays were washed with BioTek Strip washer with 1×PBS containing 0.1% (v/v) Tween-20. The slides were then removed from slide adaptors, transferred to 50 ml conical tube containing 1×PBS with 0.1% (v/v) Tween-20. The slides were dip washed twice with 1×PBS with 0.1% (v/v) Tween-20 followed by final wash with 1×PBS buffer. After the final wash, slides were transferred to fresh 50 ml conical tube and centrifuged at 1000 g for 30 sec. for drying.

### (g) Image Acquisition and Data Analysis

The assayed microarray slides were scanned at 10 μm resolution using the GenePix 4300 A Microarray Scanner (Molecular Devices, CA). Image analysis was performed using GenePix Pro7 (Molecular Devices, CA).

The approximate detection limits measured for the above assays are summarized in Table 1 below.

TABLE 1
Limit of detection (LOD) of IL-6 antigen (pg/mL)

| Antigen | Reaction | LOD on | LOD on |
|---|---|---|---|
| incubation time | medium | POEGMA | e-POEGMA |
| 4 hr | FBS/PBS | 1 | 10⁻² |
| 1 hr | FBS/PBS | 1* | 10⁻¹ |
| 1 hr | Plasma | 100 | 1 |
| 1 hr | Serum | 100 | 1 |

*Results with incubation time of 35 and 80 minutes.

### Example 3: Infectious Disease Antigen Array

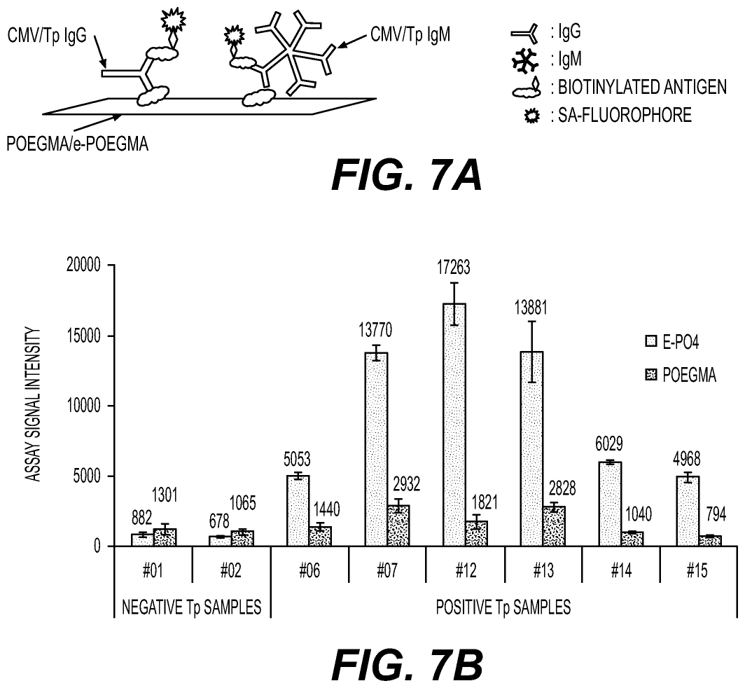

Arrays of recombinant antigens that can react with antibodies specific to Treponema pallidum (TP), a bacterium causing Syphilis infection, were printed onto POEGMA and e-PO4. An exemplary double-antigen bridge immunoassay is illustrated in FIG. 7A. The printed antigen array was incubated with samples containing the TP antibodies. The antibodies were captured on the array and then detected using a biotin-labeled recombinant antigen that can be bound by the antibodies captured on the protein microarray. In the final step, the antigen-antibody complexes were stained with cy5-labeled streptavidin. The experimental details are outlined herein below.

The results obtained for POEGMA and e-PO4 are illustrated in FIG. 7B. TP positive and negative samples were obtained from a Syphilis Accuset performance panel (SeraCare Life Sciences, cat #0820-0214). For the TP positive samples, the signal from e-PO4 was about six times (6×) higher than the signal detected from POEGMA. For the TP negative samples, the signals from e-PO4 were about half of the signals from POEGMA. Thus, positive and negative samples could be distinguished using e-PO4 coated substrates. POEGMA substrates were not able to distinguish positive samples #06, #14, and #15 from the negative samples.

The following is an exemplary experimental procedure for the double-antigen bridge immunoassay to detect TP-specific antibodies.

### (a) Antigen Microarray Printing

TP antigen R01549 was printed onto substrates using a SCIENION non-contact piezo-dispenser. The printing solution contained 0.25 mg/mL R01549 in PBS.

### (b) Post-Print Incubation

The protein microarrays were incubated on the printer stage for 1 hr at a relative humidity of 50-55%, immediately following printing. Subsequently, the microarray slides were incubated overnight at 40° C. in a slide dryer, SHURDry™ Slide Dryer II (General Data Healthcare, Fisher Scientific) to facilitate immobilization of printed antibodies. The slides were stored in sealed pouches containing a desiccant at 2-8° C.

### (c) Assay Procedure

(1) Samples were diluted (1:50) in PBS.
(2) Incubate 2 hrs on the orbital shaker at RT, 100 RPM.
(3) Aspirate sample solution and add biotinylated detection antigen R01549 (5 μg/mL). Incubate 1 hr on the orbital shaker at RT, 100 RPM.
(4) Aspirate and add AF647 labeled streptavidin (5 μg/mL) and incubate 30 min on the orbital shaker at RT, 100 RPM.
(5) Aspirate and wash by using a BioTek washer in PBS-T 0.1%.
(6) Aspirate (pipette) the excess liquid. Remove slide from the well assembly.
(7) Move to 50 mL conical tube filled with PBS-T 0.1%.
(8) After 10 sec, transfer the slide to a second 50 mL conical tube with PBS-T 0.1% in total 3 times.
(9) After rinse in the PBS-T 0.1%, transfer the slide to a fresh, dry conical tube and centrifuge at 1000×g for 30 sec.

### (d) Image Acquisition and Data Analysis

The microarray slides were scanned at 10 μm resolution using the GenePix 4300 A Microarray Scanner (Molecular Devices, CA). Image analysis was performed by using the GenePix Pro7 software (Molecular Devices, CA).

### Example 4: Antibody Staining Assay

The following provides an exemplary assay protocol for an antibody staining assay to examine the quality of protein microarrays printed on POEGMA substrates:

### (a) Mouse Anti-Human IgM Microarray Printing

Mouse anti-human IgM antibodies (anti-A or anti-B) were printed onto e-POEGMA and POEGMA substrates using a non-contact piezo-dispenser (SCIENION AG, Berlin, Germany). The printing solution contained 0.5 mg/mL antibody in PBS with 0.01% (v/v) Tween-20.

### (b) Post-Print Incubation

The protein microarrays were incubated on the printer stage for 1 hour at a relative humidity of 50-55%, immediately following printing. Subsequently, the microarray slides were incubated overnight at 40° C. in a slide dryer, SHURDry™ Slide Dryer II (General Data Healthcare, Fisher Scientific) to facilitate immobilization of the printed antibodies. The microarray slides were stored in sealed pouches containing a desiccant at 2-8° C.

### (c) Pre-Assay Wash

Prior to the assay, the microarray slides were mounted and washed with PBS containing 0.1% Tween-20. The slides were used immediately after washing without drying in-between.

### (d) Antibody Staining

100 μL of Cy3-labeled goat a mouse IgM (anti-A or anti-B Ab) was prepared in buffer and added to the arrays on the slides, and then incubated at room temperature on an orbital shaker (100 RPM) for 30 minutes.

### (e) Post-Assay Wash

After immunostaining, the microarrays were washed with PBS containing 0.1% (v/v) Tween-20. The slides were then removed from slide adaptors, transferred to 50 ml conical tubes containing PBS with 0.1% (v/v) Tween-20. The slides were dip washed twice with PBS with 0.1% (v/v) Tween-20 followed by a final wash with PBS buffer. After the final wash, slides were transferred to fresh 50 ml conical tubes and centrifuged at 1000 g for 30 sec. for drying.

### (f) Image Acquisition and Data Analysis

The assayed microarray slides were scanned at 10 μm resolution using a GenePix 4300 A Microarray Scanner (Molecular Devices, CA). Image analysis was performed using GenePix Pro7.

### Example 5: Red Blood Cell Antigen Typing

Red blood cell (RBC) typing assay protocols have been established for traditional POEGMA surfaces for both open well and closed well formats. Microarrays for the RBC typing assay rely on printing antibodies against specific red blood cell antigens of interest onto the POEGMA-coated surface, which are stably attached after a drying period and remain functional, capable of capturing RBCs as they come in contact with the array surface. A typical typing protocol involves a pre-assay wash with PBST, followed by incubation of 3% RBC suspensions for at least 5 minutes at room temperature (RT; 21-25° C.) and a PBS wash step to remove unbound cells. Image quantification is performed to determine the spot pixel intensity for each antibody type, which clearly identifies positive versus negative results. A positive result confirms surface antigen expression of a given antigen, whereas the lack of antigen in the surface of the RBCs should provide a negative result.

To compare the Epoxy-co-POEGMA substrates with traditional POEGMA, several side-by-side experiments were performed, as summarized in Table 2 below. Approximately 175 Epoxy-co-POEGMA slides have been printed with anti-RBC antibodies, of which about 70 have been used for immunostaining and/or actual typing assays. Table 2 highlights experiments with side-by-side comparisons with regular POEGMA or studies with a high number of replicate samples.

TABLE 2
Summary of typing assay experiments comparing Epoxy-co-POEGMA
and traditional POEGMA

| # | Experiment | Results |
|---|---|---|
| 1 | Direct comparison of Epoxy | Similar typing results for E-P and R-P; |
| | and Regular POEGMA for | blocking is not required |
| | RBC typing assays: #1 | |
| 2 | Direct comparison of Epoxy | Similar typing results for E-P and R-P; |
| | and Regular POEGMA for | increased Ab retention in E-P; mock |
| | RBC typing assays: #2 | BDP assays don't affect retention |
| 3 | Direct comparison of Epoxy | Noticed “rough”/“dirty” E-P substrates, |
| | and Regular POEGMA for | but “defects” didn't impact assay results |
| | RBC typing assays: #3 | |
| 4 | Typing Assay Confirmation | Reproducible results were obtained |
| | Study #2 | across donors by comparing replicates |
| | | from the same day and different days. |
| 5 | Test of the Plexus BioWell | Mean intensities varied between donors, |
| | version 1 for typing assays | regardless of having similar phenotype. |
| | | This donor-dependent variability has |
| | | been observed before, likely |
| | | independent of BioWell. |

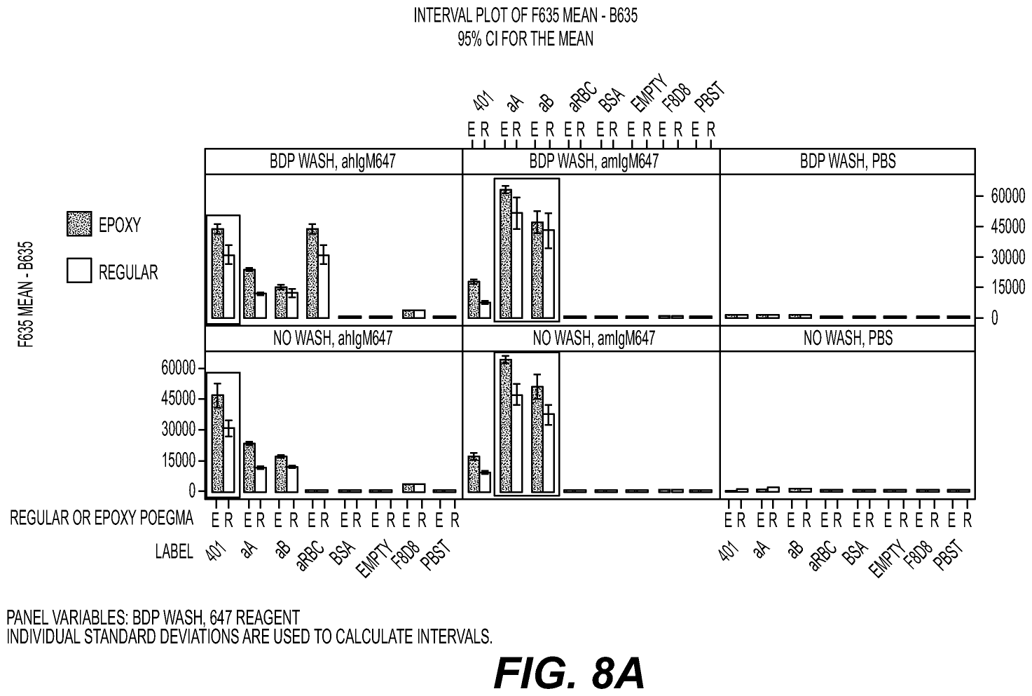

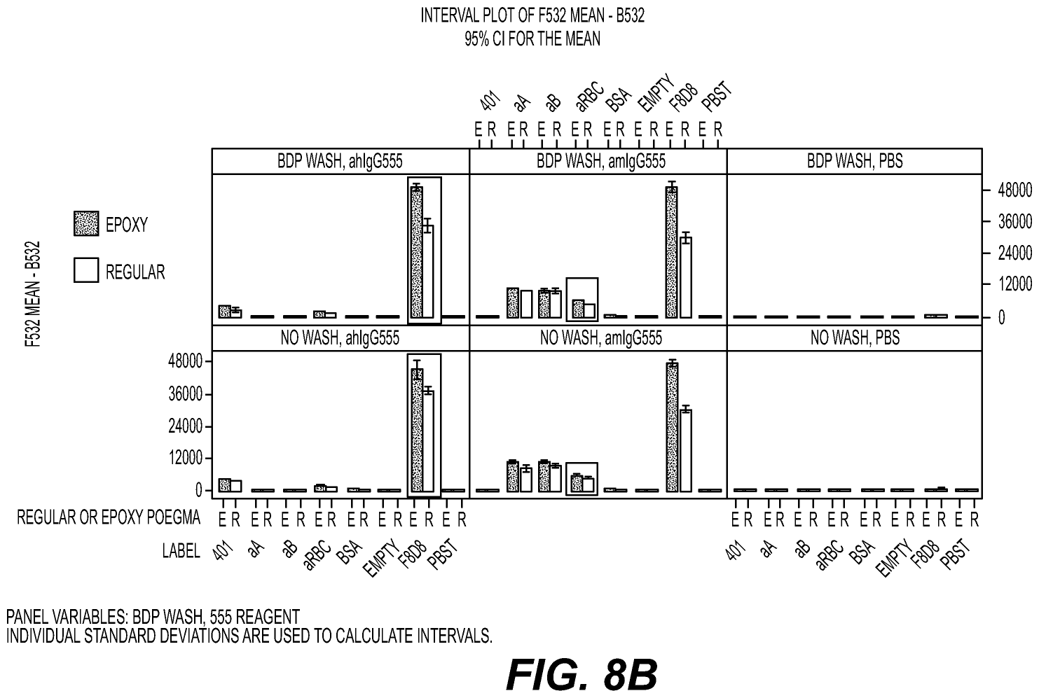

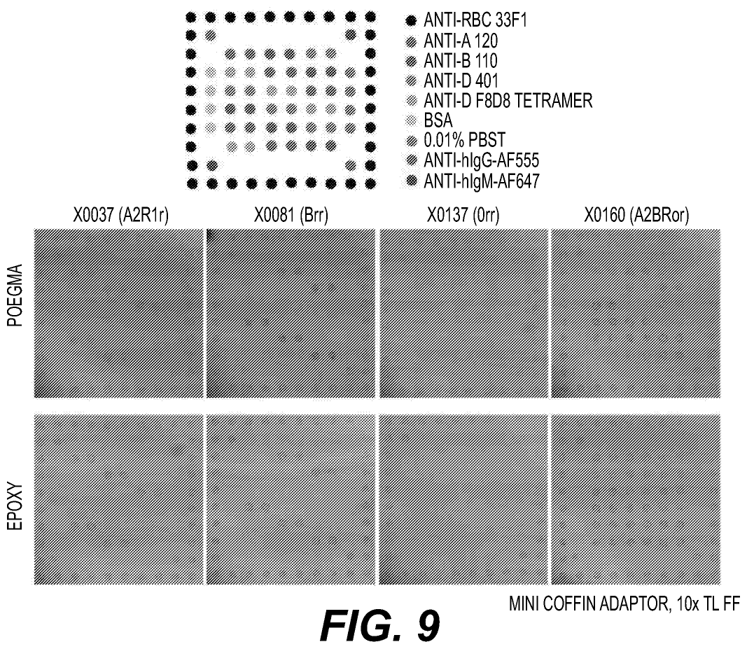

To compare e-POEGMA substrates with traditional POEGMA, the studies focused on two main parameters: (1) the retention of anti-RBC antibodies on each surface (FIGS. 8A and 8B) and (2) the results of RBC typing assays as measured by spot intensity analysis (FIG. 9). As shown in FIGS. 8A and 8B, an increase in antibody retention was observed for e-POEGMA substrates when compared to POEGMA. Nonetheless, the typing assay results for both surfaces were very similar for the tested donor cells.

In conclusion, Epoxy-co-POEGMA constitutes a viable surface chemistry compatible with microarray RBC typing assays.

### Example 6: Antibody Typing/Reverse Typing

Antibody typing/reverse typing assay involves immobilized red cells or immobilized antigens to capture antibodies (IgG or IgM) in plasma or serum. These antibodies are then detected, e.g., using secondary antibodies labeled with fluorescent molecules or probes for colorimetric detection. The performances of e-POEGMA and POEGMA were evaluated using an antibody typing assay format, e.g., with respect to the following criteria: (i) POEGMA substrate background after it is exposed to plasma and bound antibodies are detected with several different secondary detection antibodies; (ii) the retention of immobilized red cells on POEGMA after undergoing assay processing steps; (iii) the sensitivity of model antibody capture (Anti D F8D8 titration study) on POEGMA substrates with physically adsorbed cell or cells monolayer; (iv) the specificity of ABO reverse typing assay using fluorescently labeled detection reagent.

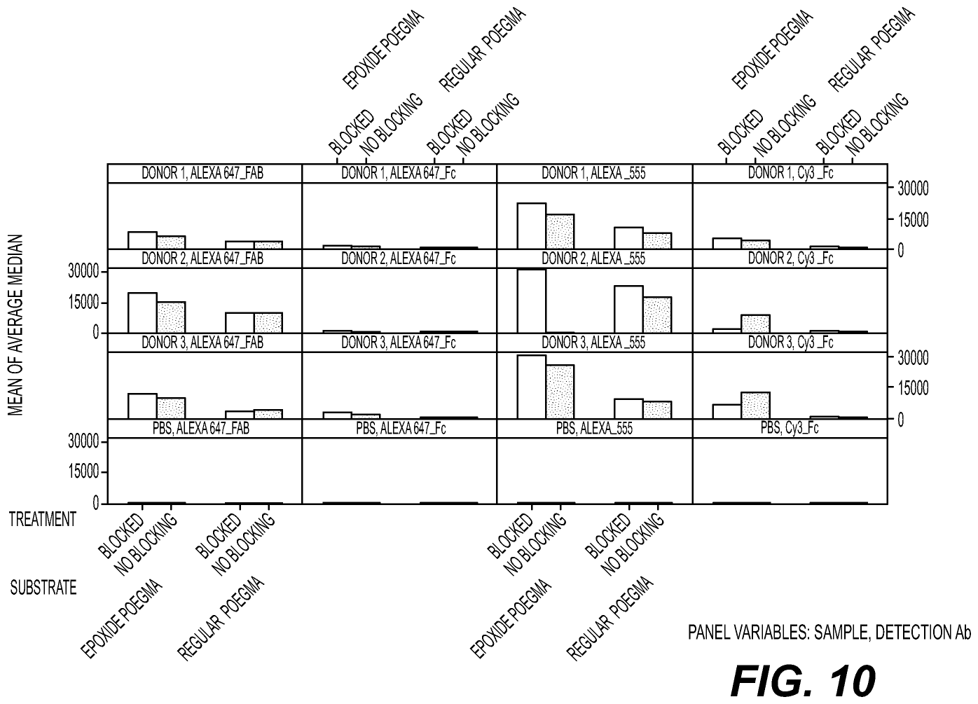

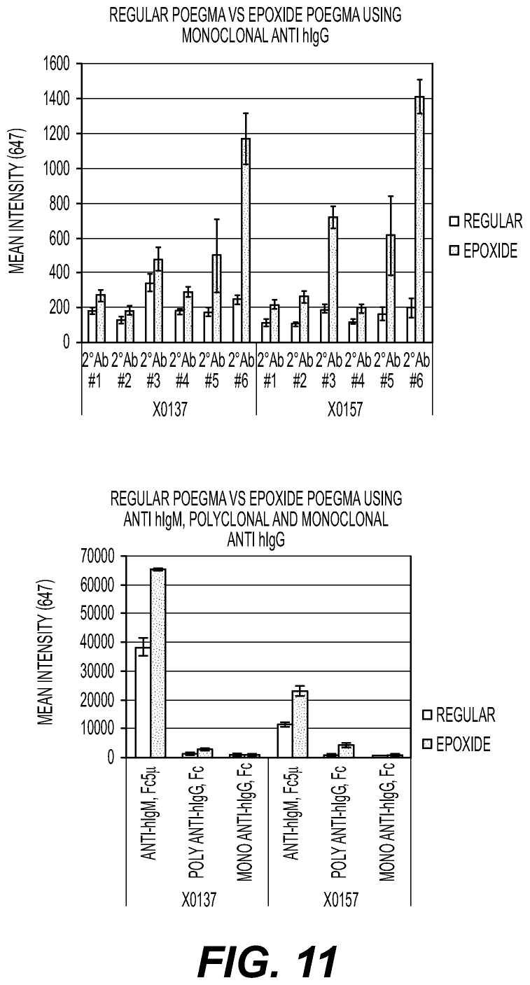

POEGMA and e-POEGMA were exposed to plasma samples from three different donors. The presence of hIgG bound to the substrate was detected using four different polyclonal detection antibodies labeled with Alexa 555, Cy3, or Alexa 647 with different specificity to Fab, Fc or whole part of hIgG. The e-POEGMA substrate generated 3-9 fold higher substrate background than regular POEGMA. The signal quantification is showed in FIG. 10. Blocking the substrate for 2 hr using Tris buffer with BSA was essentially ineffective. A similar study was carried out with plasma from two different donors using several monoclonal anti hIgG and polyclonal anti hIgM. The e-POEGMA generated 2-7 fold higher background signals than regular POEGMA. The polyclonal detection antibodies generated 3-20 fold higher background signals than the monoclonal detection antibodies. Further, the anti hIgM antibodies generated 10-200 fold higher background intensities than the anti hIgG detection antibodies. A representative result is shown in FIG. 11.

Immobilization of Red Cells on e-POEGMA Vs. Regular POEGMA

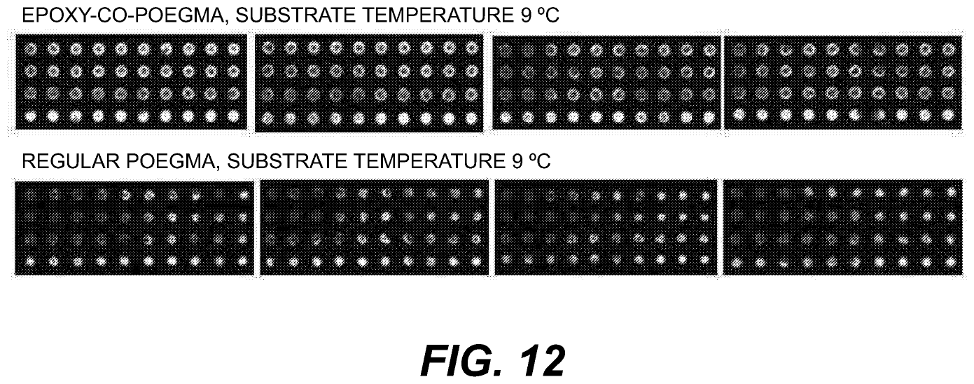

Immobilization of red cells on POEGMA can be achieved using different methods of immobilization. For example, direct physical adsorption and affinity capture using an anti RBC tetramer can be used. Immobilization of red cells directly onto e-POEGMA resulted in a more uniform spot morphology with spots that are more circular and with higher cell retention when compared to immobilization of cells onto regular POEGMA (FIG. 12). In this method, the immobilization of red cells on e-POEGMA is likely facilitated by physical adsorption and some covalent binding between primary amino groups on the cells with the epoxide groups of the POEGMA brush.

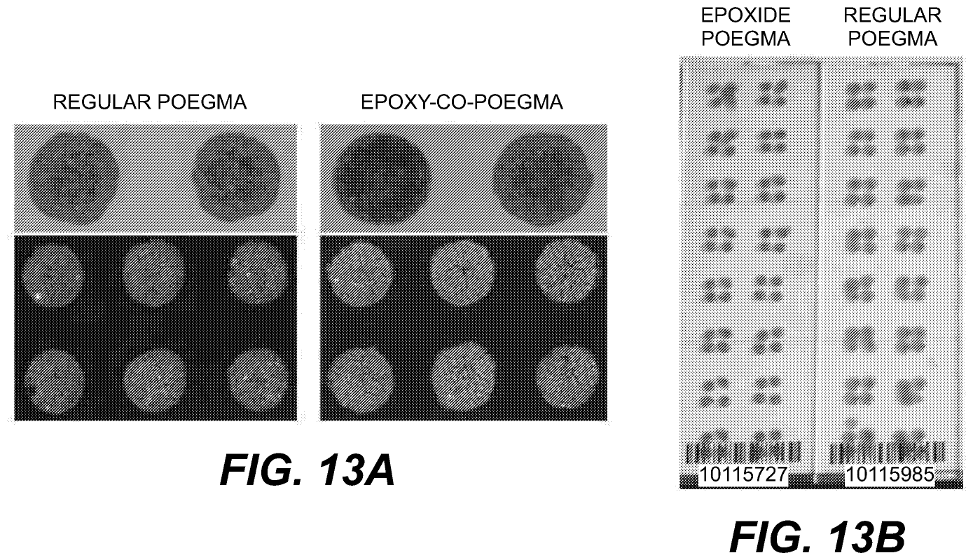

Immobilization of red cells through affinity binding to immobilized anti-RBC tetramer on e-POEGMA or regular POEGMA resulted in similar spot morphology and red cell retention (FIG. 13A). In this method, immobilization of red cells is governed by the interaction of the immobilized anti RBC tetramer with the red cell. The similarity in the immobilized cell morphology in FIG. 13A suggests that the tetramer immobilization on e-POEGMA and regular POEGMA has generated similar protein adsorption and conformation on the POEGMA brush. Although the cell morphology was similar when the POEGMA substrate was cooled to the dew point to prevent the cells spot from drying out, e-POEGMA has less condensation on the substrate and allows the cell spots to localize more accurately. This can be caused by the more hydrophobic surface of e-POEGMA compared to regular POEGMA (see FIG. 13B). As a side effect of epoxide reactive moieties, some non-specific binding of red cells on the e-POEGMA substrate was occasionally observed and a more robust rinsing technique was needed when using an indirect red cell immobilization strategy.

Anti D F8D8 Titration on e-POEGMA Vs. Regular POEGMA

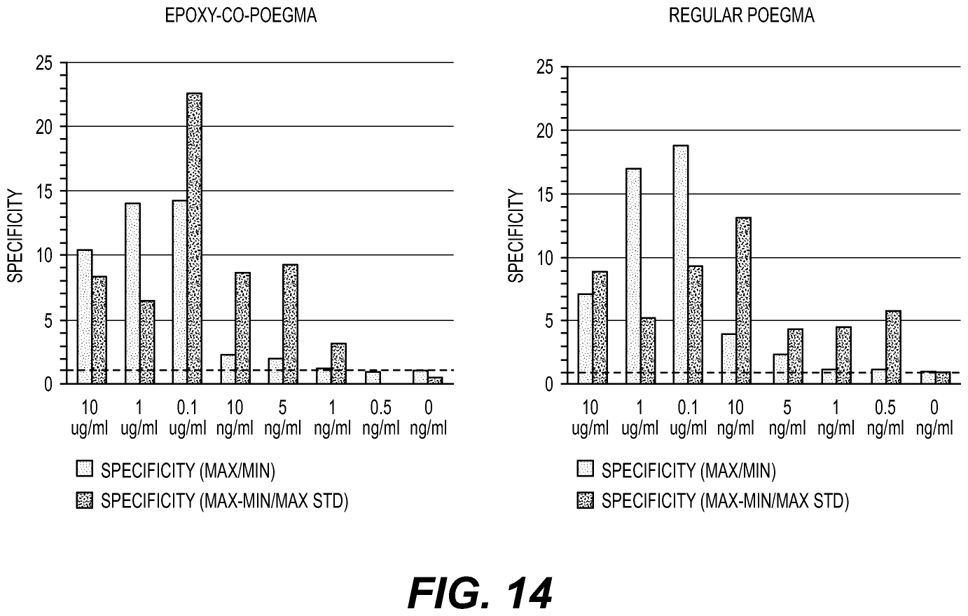

The sensitivity for detecting anti-RhD antigen antibodies (anti-D) on e-POEGMA and regular POEGMA was determined by exposing immobilized cells to titrations of anti D F8D8 antibody. The bound F8D8 is then detected with labeled detection antibody (Alexa 647 labeled anti hIgG). The sensitivity of anti D detection is defined as the limit of detection (LOD) where signal differences between positive and negative cells can still be differentiated using a specificity value. Specificity is calculated by taking the ratio of signal intensity on the D positive cells (Max signal) to the D negative cells (Min signal). As shown in FIG. 14, the LOD of anti D detection (diluted in PBS+2% BSA) is determined as the lowest concentration of anti D where specificity >1. On regular POEGMA the LOD of anti D detection is 500 pg/ml while e-POEGMA it is 1 ng/ml, which is comparable for the two substrates. Another method to calculate specificity is to include the standard deviation of the signal in the calculation, i.e. to take the differences of Max and Min signal and normalizes the difference with the deviation of the Max signal, which resulted in similar LODs.

The following is an exemplary protocol used to study the plasma background and anti D titration.

### (a) POEGMA Slide Preparation

Blank slides were used for the plasma background study. Cell monolayers were immobilized on e-POEGMA and regular POEGMA for the anti D titration assay. Cell monolayers were immobilized on a printed tetramer spot comprised of a mixture of anti RBC, 33F1 (Immucor) at 0.125 mg/ml and anti mIgG (Jackson) at 0.125 mg/ml. The printed slide was incubated overnight in a vacuum dehumidifier at RT before deposition of cells on the printed tetramer spots. Appropriate red cells at 9-12% concentration were deposited at 0.5 μl to cover 4×4 microarray spots and incubated for 30 min on a stage that is cooled to the dew point (˜10° C.) before rinsing with 0.1% PBST. The slide with immobilized red cells was then dried by centrifugation at 800 g for 30 s twice. Slides were then stored in a vacuum dehumidifier at RT until they were used.

### (b) Plasma Background Determination

The slide was first washed with 0.1% PBST, which then aspirated before addition of the blocking buffer (1% BSA-10 mM Tris-0.01% T-20, pH˜7.5). Blocking was done for 2 hr at RT on a rotator (150 rpm) on applicable wells and then washed with 0.1% PBST. The entire wash buffer was then aspirated and 100 μl of non-diluted plasma (freshly thawed from −20° C.) was added into the wells. Plasma was incubated on the substrate for 35 min at RT and then washed with 0.1% PBST on a Biotek washer. Secondary antibodies (100 μl, 10 μg/ml) were added and incubated on the substrate for 20 min at RT. The slide was then washed again with 0.1% PBST and spin dry.

### (c) Anti D Titration

The slide was first washed with 0.1% PBST, which then aspirated before the addition of anti D F8D8 samples. Plasma (X0137) spiked with anti D F8D8 at different concentration (500 pg/ml to 10 μg/ml) was incubated on the substrate for 21 min at 37° C. on a P2 incubator and then wash with 0.1% PBST on a Biotek washer. Secondary antibody (goat anti hIgG, (Fab2) fragment, Fc specific, Alexa 647 (Jackson), 100 μl, 10 μg/ml) were added and incubated on the substrate for 20 min at RT. The slide was then washed with 0.1% PBST and spin dry.

### (d) Image Acquisition and Data Analysis

Microarray slides were scanned at 10 μm resolution using a GenePix 4300 A Microarray Scanner (Molecular Devices, CA). Image analysis was performed using GenePix Pro7 (Molecular Devices, CA).

### Example 7: ABO Reverse Typing

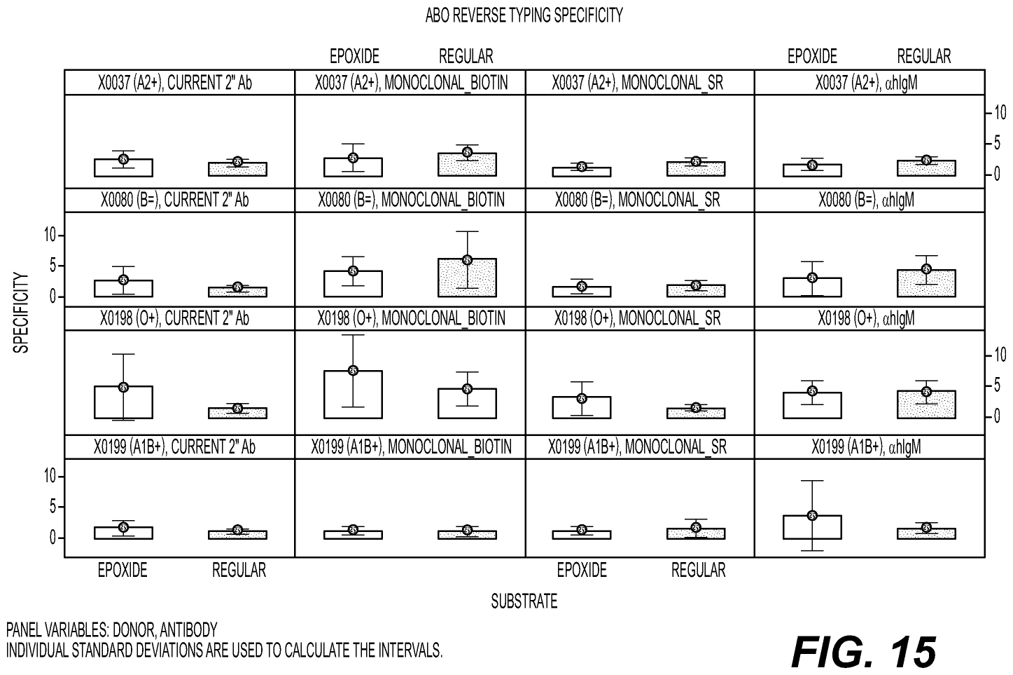

ABO reverse typing was performed with cells immobilized on e-POEGMA and regular POEGMA for detecting the presence of circulating antibodies in plasma. Plasma from four different donors were analyzed using polyclonal anti hIgG, monoclonal anti hIgG and polyclonal anti hIgM as the detection antibodies. The comparison of ABO reverse typing specificity across different detection antibodies and four donor's plasma is shown in FIG. 15. Specificity was determined by taking the ratio of the positive cells signal to the negative cells signal. The data showed no significant difference in the performance of e-POEGMA vs. regular POEGMA. The ABO reverse typing specificity is relatively low due to high signal intensities generated by the negative cell spots. Overall, e-POEGMA gave results comparable to regular POEGMA in terms of assay specificity for antibody screening or ABO reverse typing. The substrate background intensity was higher on e-POEGMA than on regular POEGMA. Nevertheless, the higher substrate background did not affect the specificity of the screening assay carried out on e-POEGMA.

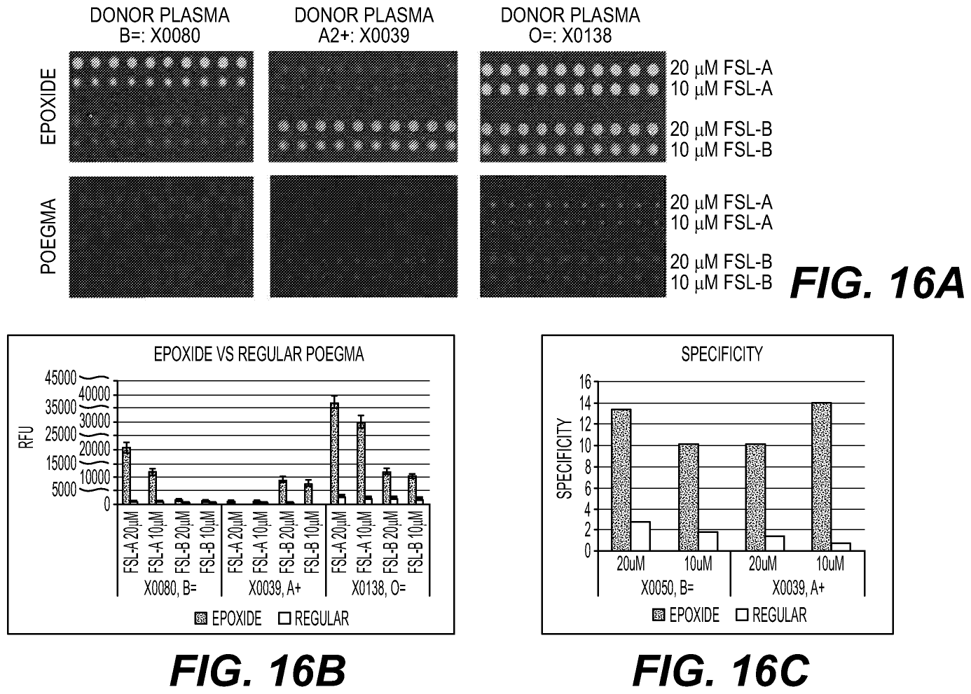

To reduce the non-specific signal on ABO reverse typing upon exposure to plasma, synthetic antigens A and B were used instead of red cells antigen. Carbohydrate molecules (mimicking A and B antigen) that are linked to a functional spacer lipid (FSL) 1,2-O-dioleoyl-sn-glycero-3-phosphatidylethanolamine (DOPE) were immobilized on POEGMA substrates. The synthetic FSL antigen adsorbed and immobilized more readily on e-POEGMA than on regular POEGMA. FIGS. 16A, 16B, and 16C show that after exposure to plasma, the signal intensity of bound anti A and anti B antibodies on e-POEGMA is significantly higher (6-24 fold) than the signal intensity found for regular POEGMA, which results in a higher specificity. For the synthetic antigen, the functional group of the e-POEGMA surface appears to increase the adsorption of the FSL molecules. An improvement in the adsorption of FSL antigens to the regular POEGMA was observed when the surface was baked before printing the synthetic antigen. A smaller improvement was observed after baking the e-POEGMA substrate.

An exemplary protocol used to study ABO reverse typing is provided herewith.

### (a) Ghost Red Cells Reagent Preparation for Printing

Donor blood samples were washed five times using red cell diluent (RCD). The cells were diluted to 3% hematocrit (1× conc) by taking 300 μL packed cells (˜80%) into 8 ml RCD. To make ghost cells for printing, 1.5 ml of 3% red cells was washed twice with PBS by centrifuging at 3000 g for 1 min and then re-suspended in 1.5 ml PBS. 50 μl of 5 mg/ml digitonin was added into the red cells and spun immediately at 4° C. at 3000 g for 40 min. The supernatant was removed and cells were washed with PBS five times. The ghost cells were reconstituted into 300 μl to get a cell concentrate of ˜15% for printing.

### (b) Ghost Cells and Synthetic Antigen Printing

Ghost cell stocks (˜15% cell concentrate, A, B, AB, 0 cells) in PBS and 10 μM, 20 μM synthetic antigen in MilliQ water were printed on the substrates using a non-contact piezo-dispenser (SCIENION AG, Berlin, Germany) at 50% relative humidity and room temperature (RT). The printed slides were kept in a vacuum dehumidifier at RT until use.

### (c) Assay Protocol

Slides were exposed to sample immediately without a pre-assay wash. 50 μl plasma was added into the empty well, followed by 100 μl PBS and incubated for 21 minutes at RT. The samples were then aspirated completely from the wells using the vacuum aspirator and subsequently 150 μl of 0.1% PBST was added into the well to prevent the drying of the substrate. The substrate was then washed with 0.1% PBST using an automated Biotek washer.

To detect binding of antibodies to the immobilized cells, 100 μl of the appropriate secondary antibodies (10 μg/ml for goat anti hIgG, (Fab2) fragment, Fc specific, Alexa 647 (Current 2° Ab), 1 μg/ml for monoclonal anti hIgG, biotin or SPR labeled, and 1 μg/ml for anti hIgM, Fcμ specific, Alexa 647) was added into each well and incubated for 10 minutes at RT in dark followed by a wash with 0.1% PBST. For biotinylated secondary antibodies, 100 μl of SAV-Alexa647 (1 μg/ml) were added and incubated for 10 min @ RT in the dark while other wells were incubated with PBST 0.1% wash solution. Slides were then washed with 0.1% PBST for a final wash and then removed from the 16-well holder and spun dry.

### (d) Image Acquisition and Data Analysis

The assayed microarray slides were scanned at 10 μm resolution using a GenePix 4300 A Microarray Scanner (Molecular Devices, CA). Image analysis was performed using GenePix Pro7 software.

### Example 8: Oligonucleotide Array and Hybridization Assay

Small molecule binding to substrates is important for printing DNA microarrays, which typically use short oligonucleotide probes. In this example, an oligonucleotide probe containing the T7 universal primer sequence was used. The probe included an amino group at the 5′ end for potential reaction with the epoxy group on the substrates, and a biotin moiety at the 3′ end for detection using cy5-labeled streptavidin. The T7 probe (IDT, Iowa) is shown below:

(SEQ ID NO: 1)
5′-AmMC6-TA ATA CGA CTC ACT ATA G Biotin-3′

An array of the probe was spotted onto POEGMA and e-PO4 slides following the procedure described below. The probe was then detected using Cy5-labeled streptavidin. The fluorescence intensities are summarized in FIG. 17A. Results indicate that e-PO4 retains the oligo probe four times (4×) more efficiently than POEGMA. Background intensities were comparable for both substrates.

DNA hybridization assays were carried out using both substrates and a fluorescently labeled oligo with a sequence complementary to the above T7 sequence. The hybridization assay signals from e-PO4 was about 100 fold higher than the signal obtained from POEGMA, which was barely above background. Results are summarized in FIG. 17B.

Even though there was a considerable amount of oligonucleotide probe deposited onto the POEGMA as shown in FIG. 17A, the probe didn't seem to participate in the hybridization reaction with the complementary probe. The lack of hybridization can be a consequence of the physical interaction of the oligo probes with the POEGMA brushes. Binding may require a relatively large contact area, which is consequently blocked and unavailable for the hybridization reaction. In addition, the hybridization reaction was carried out at higher temperature (53° C. for hybridization and room temperature for streptavidin staining). The post hybridization assay wash was also more stringent (2×SSC Buffer with 0.2% SDS) than that for streptavidin staining (1×PBS with 0.1% Tween-20). It is likely that the stringent reaction and washing conditions could more easily remove the oligo probes that were adsorbed to the substrate. The Streptavidin staining procedure follows the immunoassay procedure as described in Example 2.

The following is an exemplary protocol used in the DNA oligonucleotide array printing and hybridization assays.

### (a) T7 Oligonucleotide Microarray Printing

A 10 μM solution of synthetic oligonucleotide with 5′-amino modified T7 primer sequence (18 nt, IDT, Iowa) was prepared in PBS with 0.5 mg/mL BSA. The oligo solution was then printed onto the polymer substrates (e.g. POEGMA coated glass slide), using a non-contact piezo-dispenser.

### (b) Post-Print Incubation

After array printing, the substrates were incubated for 1 hour at a relative humidity of 50-55%. Subsequently, the microarray slides were transferred to the slide dryer, SHURDry™ Slide Dryer II (General Data Healthcare, Fisher Scientific), and incubated at 40° C. overnight to immobilize the oligos on the substrate surface. After incubation, the printed slides were stored in sealed pouches containing a desiccant at 2-8° C.

### (c) Pre-Assay Wash

Prior to the hybridization assay, the DNA oligo microarray slides were mounted and washed with 2×SSC solution 3 times. The slides were immediately used after washing without drying the surface.

### (d) Hybridization

A 5 μM solution of synthetic 5′-Cy5-labeled oligonucleotide (with T7 complementary sequence) in hybridization buffer containing 3×SSC-0.1% SDS was added to the microarrays on the mounted slides. Hybridization reaction was carried out in a humidity-controlled chamber at 53° C. for 30 min.

### (e) Post Hybridization Wash

After hybridization, the microarray wells were washed once with 2×SSC buffer containing 0.2% (v/v) SDS for 10 minutes followed by 2×SSC Solution for 10 min and then 0.2×SSC for another 10 min. After the final wash, slides were demounted and transferred to a fresh 50 mL conical tube and centrifuged at 1000×g for 30 sec. for drying.

### (f) Image Acquisition and Data Analysis

The oligonucleotide microarray slides were scanned at 10 μm resolution using a microarray scanner. Image analysis was performed using the GenePix Pro7 software (Molecular Devices, CA).

### Example 9: Comparison of e-POEGMA with Commercial Epoxy Slides

Epoxy functionalized glass slides are commonly used to create protein and DNA microarrays. In this study, e-PO4 coated slides were compared with two brands of epoxysilane coated slides, SuperEpoxy 2 (ArrayIT, Sunnyvale, Calif.) and NEXTERION® Slide E (Schott, Germany).

### Sensitivity/Limit of Detection

First, IL-6 assay sensitivity for the different slides was compared. For the IL-6 assay, the model conditions described in Example 2 were used, except that the IL-6 antigen incubation time was shortened to 1 hour.

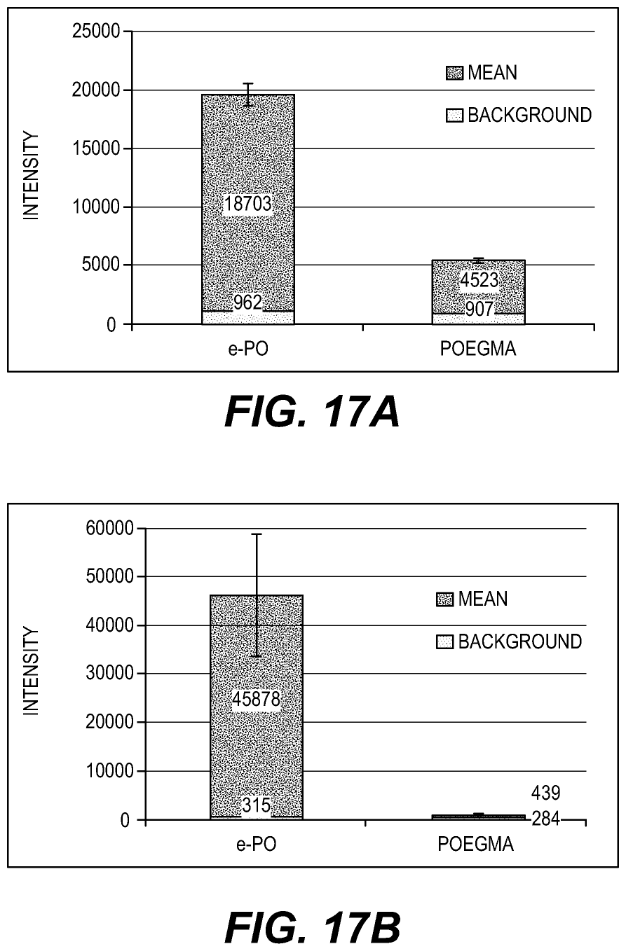

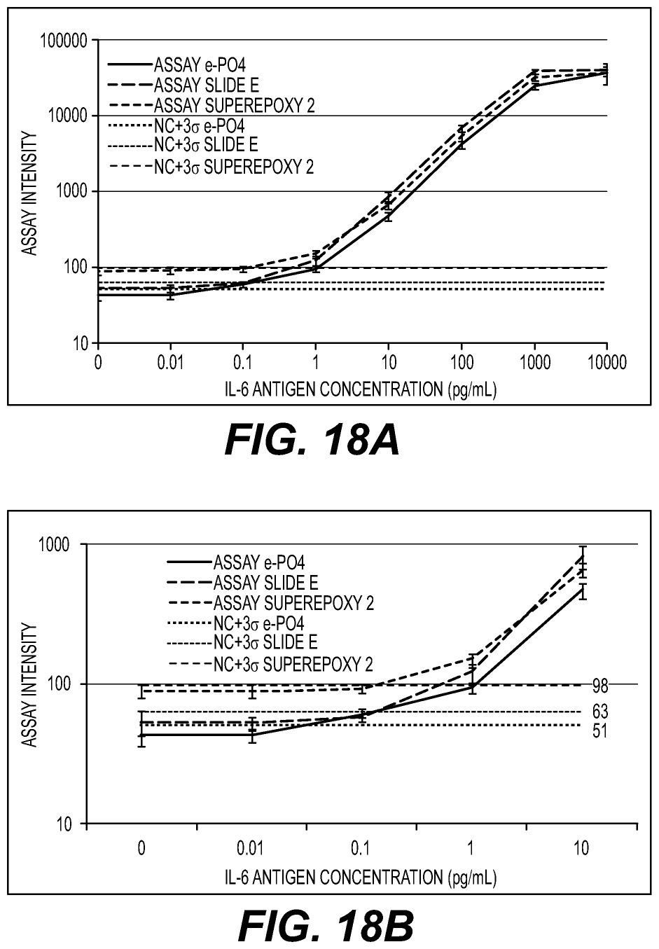

To establish the limit of detection (LOD), a cut-off intensity was calculated as the mean intensity from the blank (NC)+3 standard deviations for each assay. The LOD cut-off increased in the order, e-PO4<Slide E<SuperEpoxy2. Signal intensities above the cut-off were considered true positive signals, and signals below the cut-off were considered noise. By this criterion, the detection limit for the assay performed on e-PO4 was about 0.1 pg/mL as the assay signal is above the cut-off at IL-6 concentration of 0.1 pg/mL and below the cut-off at 0.01 pg/mL. Similarly, the detection limits for Slide E and SuperEpoxy2 were about 0.1-1 pg/mL. The results are shown in FIGS. 18A and 18B.

### Binding Capacity

Binding capacity is an important parameter for comparing microarray substrates. Since the epoxide group of the e-POEGMA reacts with primary amines, a fluorescently labeled primary amine, FITC-PEG-NH₂ (MW˜2 k, Creative PEGWorks, Chapel Hill, N.C.) was used to estimate the binding capacity for e-PO4 and the two commercial epoxysilane slides. FITC-PEG-NH₂ is soluble in water and the molecule can be quantified based on the fluorescence intensity from the FITC fluorophore. For e-PO4 and POEGMA, two coating thicknesses were selected: ˜20 nm (typical) and ˜70 nm (high end).

The experiment was carried out by incubating each slide in a 0.5 mg/mL solution of FITC-PEG-NH₂ with sodium tetraborate/hydrochloric acid buffer (pH 9) for 3 different incubation times: 1 hour, 18 hours, and 8 days. Each time after incubation, the slides were rinsed sequentially in 6 containers with freshly prepared DI water, and dried by centrifugation. The fluorescence images for the slides were obtained using a GenePix 4300 A Microarray Scanner (Molecular Devices, CA). The fluorescence intensity from the slide image was converted to the amount of FITC-PEG-NH₂ per area by referring to a standard curve. The standard curve was derived from a reference slide that had a series of spots with different known amounts of FITC-PEG-NH₂.

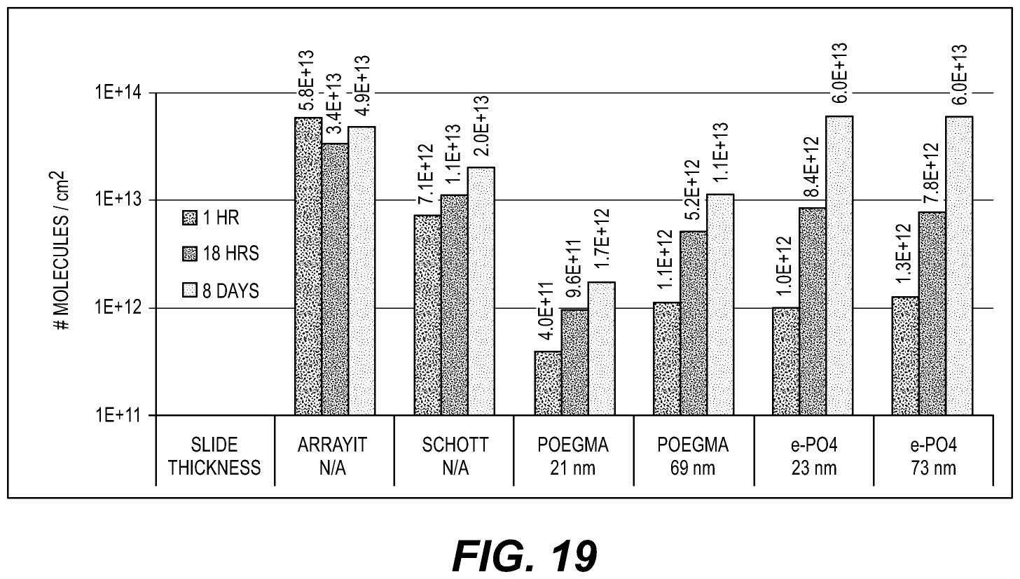

The binding capacities for all slides are summarized in FIG. 19 and as follows:

(1) The number of bound molecules increased with incubation time for all slides except SuperEpoxy2.
(2) After incubation for 8 days, the binding capacities were ranked from high to low as the following: e-PO4 (23 nm)≈e-PO4 (73 nm)>ArrayIT>Schott>POEGMA (69 nm)>POEGMA (21 nm).
(3) e-PO4 had the most dramatic increase of molecular binding over an extended incubation time. From 1-hour to 8-day, the binding capacity increased about 50 fold to reach a final binding capacity of about 6.0×10¹³ molecules/cm², equivalent to 1.67 molecule/nm², or ˜5×10⁵ molecules for a microarray spot size of 150 μm in diameter.
(4) SuperEpoxy2 surface seemed to be saturated at 5.8×10¹³ molecule/cm² after one hour incubation. The apparent binding capacity slightly decreased after further incubation.
(5) POEGMA has the lowest binding capacity among the slides. With 8-day incubation, the 21 nm POEGMA had a binding capacity 1.7×10¹² and the 69 nm POEGMA had a binding capacity 1.1×10¹³ molecule/cm². The thicker POEGMA (69 nm) has 6-fold higher binding capacity than the thinner POEGMA (21 nm).

The main difference between e-PO4 and the two commercial epoxysilane slides was the time dependency of the binding capacity. In this experiment, the substrates were submerged in a solution of FITC-PEG-NH₂, which needed to diffuse towards the epoxide on the substrate before reaction. For the commercial epoxysilane coated slides, all epoxide groups were on the very top surface of the substrate, and the FITC-PEG-NH₂ molecules from the solution readily reacted with these epoxide groups to form covalent bonds. For e-PO4, it is likely that most of the epoxide groups were buried underneath the top surface, and that time was needed for the FITC-PEG-NH₂ molecules to penetrate the polymer brush and reach the epoxide groups for covalent binding. Thus, a longer incubation was needed to immobilize the probes on the e-PO4 substrate.

Unlike the process for molecular binding in solution, the microarray printing process directly spots the probes on the substrate and the probes are then dried to immobilize. The drying process can force the printed probes to contact the substrate surface and help with the attachment of the probes on the substrate.

POEGMA substrates had some finite binding capacity even though they don't have any reactive functional group for covalent bonding. However, their binding capacity was about one order of magnitude lower than that of e-PO4. The non-covalent interaction could also explain the observation that thicker POEGMA had higher binding capacity than thinner POEGMA because the FITC-PEG-NH₂ molecule could be more entangled with the longer brushes from the thicker POEGMA.

It is possible that the surface binding capacity for FITC-PEG-NH₂ is saturated at 6.0×10¹³ molecule/cm². At this binding level, each bound molecule occupies an area less than 1 nm², which is considerably crowded for a molecule with molecular weight of 2000. Further binding study with smaller molecules may help to understand more details of the binding capacity. The binding capacity saturation could also explain the observation that thicker e-PO4 had comparable binding capacity as thinner one. On the other hand, the thicker POEGMA substrate showed higher binding capacity than the thinner one, because the POEGMA binding capacity is far from saturation for both substrates.

e-POEGMA based microarray outperforms POEGMA based microarray in immunoassays such as IL-6 antigen detection and TP antibody detection, in terms of absolute assay signal and detection limit. In addition, unlike POEGMA, e-POEGMA can also be a substrate for microarray DNA hybridization assay. In comparison with commercial slides coated with epoxysilane, e-POEGMA has comparable binding capacity and lower background, attributable to the POEGMA-like non-fouling characteristics.

In terms of red blood cell antigen typing, e-POEGMA constitutes a viable surface chemistry compatible with microarray RBC typing assays. While anti-RBC antibody retention is slightly higher in e-POEGMA, both types of coatings produce equivalent typing results.

In terms of red cells immobilization, e-POEGMA performed better than when cells are physically adsorbed on the polymer brush, showing a more uniform spot morphology and a better cells retention after multiple wash. However, when cells were immobilized through affinity capture by the anti RBC tetramer, similar immobilized cells morphology on regular and e-POEGMA was observed. To simulate the screening assay, the sensitivity of anti D detection with relevant red cells immobilized on regular versus e-POEGMA was determined. It was found that both substrates performed similarly and displayed similar sensitivity. One of the significant differences observed was the higher substrate background generated by e-POEGMA (2-7 fold higher compared to regular POEGMA) when it was exposed to plasma or serum, although the background is low enough to allow signal differentiation of negative and positive signal. Overall, there is no significant advantage of using e-POEGMA for red cells immobilization and antibody screening. On the other hand, immobilization of synthetic antigen on Epoxy-co-POEGMA resulted in an ABO reverse typing assay that had significantly higher specificity compared to the regular POEGMA due to better adsorption and immobilization of the synthetic antigen on Epoxy-co-POEGMA.

### Example 10: Reducing Background Fluorescence

It was investigated whether adding polyethylene glycol (PEG) of different sizes and end groups to plasma samples would reduce the background signal previously observed from these plasmas on an epoxy POEGMA substrate.

Prior experiments indicated that some donor plasmas produce a very high background fluorescence signal when using a fluorescently-labeled anti-human IgM as a secondary antibody in reverse typing assays on an epoxy POEGMA substrate. It has been suggested in the literature that the plasma of up to 72% of people may contain anti-PEG (18% IgG, 25% IgM, and 30% both IgG and IgM, Yang et al. Anal. Chem. 2016, 88, 11804-11812).

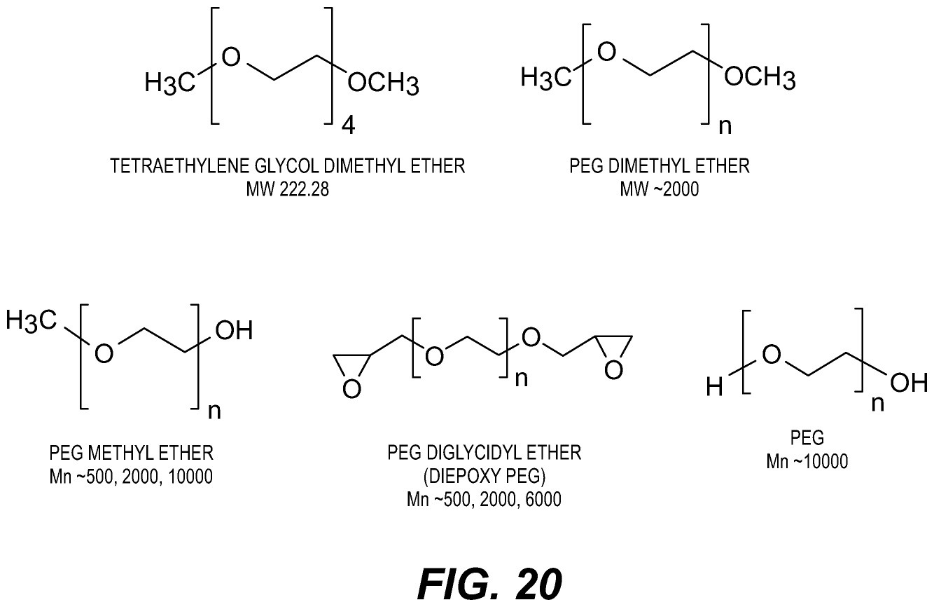

Therefore, it was hypothesized that the high background signal observed on the epoxy POEGMA substrate was due to the binding of anti-PEG in the plasma to the POEGMA surface. In this experiment, PEG was spiked into plasma samples prior to exposing the plasma to the substrate, in an effort to saturate the binding sites of any anti-PEG that may be in those plasma samples, and thus reduce any background signal caused by the binding of anti-PEG to the epoxy POEGMA surface when using Alexa 647-labeled anti-hIgM as the secondary antibody. The molecular weight and end groups of the PEG were varied to determine what effect, if any, these had on background reduction (FIG. 20). Some PEGs were chosen in an effort to mimic the surface of the epoxy POEGMA surface.

In addition, the effects of background reduction on the ability to observe the activity of synthetic antigens FSL-A and FSL-B were also studied.

I. Materials and Reagents

Two slides were used in this experiment. The information about the slides is provided in Table 3 below. The substrate used in this example was Epoxy POEGMA. The print reagent was FSL-tetra A, 50 μM in water, or FSL-tetra B, 200 μM in water, both manufactured by KODE. Information on the 6 plasma samples from in-house donors is provided in Table 4. Alexa 647-labeled F(ab′)2 fragment goat anti-human IgM, Fc_{5μ}, fragment specific (1 μg/ml in PBS, Jackson ImmunoResearch, PA, Catalog Number: 109-606-129, Lot Number: 123493) was used as detection reagent. PEG reagents are provided in Table 5. The nine varieties of PEG used in this experiment are illustrated in FIG. 20.

TABLE 3
Slides Information

| # | Slide | Thickness | Substrate | Array Details | Comments |
|---|---|---|---|---|---|
| 1 | WRD170 | 20.3 nm | Epoxy | No spots | |
| | −15 | | POEGMA | | |
| 2 | WRD163 | 25.3 nm | Epoxy | 2 sets of 2 × 10 | Stored in |
| | −18 | | POEGMA | FSL-A(50 uM) | desiccator |
| | | | | and FSL-B | after print |
| | | | | (200 uM) | |

TABLE 4
Plasma samples

| # | Anticoagulant | ABO | Rh |
|---|---|---|---|
| 1 | EDTA | A1+ | R1R1 |
| 2 | EDTA | A2+ | R1R2 |
| 3 | EDTA | B+ | R2r |
| 4 | EDTA | B+ | Rir |
| 5 | EDTA | A1B+ | R1R1 |
| 6 | EDTA | O= | rr |

TABLE 5
PEG Reagents

| | | | Catalog |
|---|---|---|---|
| # | Reagent | Manufacturer | Number |
| 1 | Tetraethylene glycol dimethyl | Aldrich | 172405- |
| | ether MW 222.28 | | 250G |
| 2 | Poly(ethylene glycol) dimethyl | Aldrich | 445908- |
| | ether avg Mn 2000 | | 50G |
| 3 | Poly(ethylene glycol) diglycidyl | Aldrich | 475696- |
| | ether avg Mn 500 | | 100ML |
| 4 | Poly(ethylene glycol) diglycidyl | Aldrich | 731811- |
| | ether avg Mn 2000 | | 5G |
| 5 | Poly(ethylene glycol) diglycidyl | Aldrich | 731803- |
| | ether avg Mn 6000 | | 5G |
| 6 | Poly(ethylene glycol) methyl | Aldrich | 202487- |
| | ether avg Mn 550 | | 5G |
| 7 | Poly(ethylene glycol) methyl | Aldrich | 202509- |
| | ether avg Mn 2000 | | 5G |
| 8 | Poly(ethylene glycol) methyl | Aldrich | 732621- |
| | ether avg Mn 10,000 | | 5G |
| 9 | Poly(ethylene glycol) avg Mn | Sigma-Aldrich | P6667- |
| | 10,000 | | 500G |

Other reagents and equipment used are provided in Tables 6 and 7, respectively.

TABLE 6
Other Reagents

| | | | Catalog |
|---|---|---|---|
| | Reagent | Manufacturer | Number |
| 1 | pHix phosphate buffer, | Immucor | 0005070 |
| | 200 ml | | |
| 2 | Blood Bank Saline, | Immucor | 23-062-125 |
| | Isotonic Solution 0.90% | | |
| | w/v, 10L | | |
| 3 | Tween 20 | Sigma | P9416-100ML |
| 4 | Red Cell Diluent (RCD), | Immucor | 0005036 |
| | Red Blood Cell Storage | | |
| | Solution | | |

TABLE 7
Equipment

| | Equipment | Manufacturer/Supplier | Catalog # | Equipment ID |
|---|---|---|---|---|
| 1 | ProPlate 16-well | Grace Bio-labs | 204862 | N/A |
| | chamber with | | | |
| | Delrin snap clips | | | |
| 2 | Biotek plate washer | Biotek | N/A | 559078 |
| | ELx50 Thermo | | | |
| | Scientific Sorvall ST | | | |
| 3 | 40R benchtop | Thermo Scientific | N/A | N/A |
| | centrifuge equipped | rotor | | |
| | with a TX-750 | | | |
| 4 | GenePix 4300A | Molecular Devices | N/A | N/A |
| 5 | Milli-Q Direct 8 | EMD Millipore | N/A | 559067 |
| | water purification | | | |
| | system | | | |

II. Protocols

### a) Buffer Preparation

To prepare PBS, 100 ml of pHix phosphate buffer was added to 10 L of blood bank saline. To prepare PBS/0.1% Tween 20 (PBS-T), 1 ml of Tween 20 was added to 1 L of PBS.

### b) Sample Processing

As mentioned above, plasma tested was obtained from in-house donors. Whole blood was centrifuged at 3000×g for 10 minutes to separate the plasma from the red blood cells. Plasma was aliquoted and stored in the freezer. Buffy coat was discarded and packed cells were washed twice with RCD before being resuspended in RCD and stored in the refrigerator.

Synthetic antigens FSL-A and FSL-B were thawed and diluted to 50 pM and 200 pM in water, respectively.

Frozen plasma was thawed and used the same day, or stored at 4° C. until further use.

Controls without PEG were prepared by adding 50 μL of plasma to 100 μL of PBS and incubating at RT for 20 min prior to the assay.

PEG stock solutions were prepared by dissolving the PEG in water at a concentration of 300 mg/ml, then diluted in PBS at a total PEG concentration of 15 mg/ml. To 100 μL of PEG solution in PBS, 50 μL of A2+(R1R2) plasma was added, for a total concentration of 10 mg/mL PEG, and the mixture was incubated at room temperature (RT) for 20 min prior to the assay (i.e., before adding it to the wells). In mixtures, PEGs were added at equal concentrations such that the total combined PEG concentration in PBS was 15 mg/mL. The protocols for making the plasma/PEG mixtures for slides 1 and 2 are shown in Tables 8 and 9, respectively.

TABLE 8
Plasma/PEG mixture protocol for each well of slide #1

| Well | Slide #1 Plasma/PEG Mixture Protocol |
|---|---|
| 1 | 95 μL PBS + 5 μL of 300 mg/ml PEG1 + 50 μL X0044 plasma |
| 2 | 95 μL PBS + 5 μL of 300 mg/ml PEG2 + 50 μL X0044 plasma |
| 3 | 95 μL PBS + 5 μL of 300 mg/ml PEG3 + 50 μL X0044 plasma |
| 4 | 95 μL PBS + 5 μL of 300 mg/ml PEG4 + 50 μL X0044 plasma |
| 5 | 95 μL PBS + 5 μL of 300 mg/ml PEG5 + 50 μL X0044 plasma |
| 6 | 95 μL PBS + 5 μL of 300 mg/ml PEG6 + 50 μL X0044 plasma |
| 7 | 95 μL PBS + 5 μL of 300 mg/ml PEG7 + 50 μL X0044 plasma |
| 8 | 95 μL PBS + 5 μL of 300 mg/ml PEG8 + 50 μL X0044 plasma |
| 9 | 95 μL PBS + 5 μL of 300 mg/ml PEG9 + 50 μL X0044 plasma |
| 10 | 100 μL PBS + 50 μL X0044 plasma |
| 11 | 95 μL PBS + 0.56 μL of 300 mg/ml PEG1 + 0.56 μL of 300 |
| | mg/ml PEG2 + 0.56 μL of 300 mg/ml PEG3 + 0.56 μL of 300 |
| | mg/ml PEG4 + 0.56 μL of 300 mg/ml PEGS + 0.56 μL of 300 |
| | mg/ml PEG 6 + 0.56 μL of 300 mg/ml PEG6 + 0.56 μL of 300 |
| | mg/ml PEG7 + 0.56 μL of 300 mg/ml PEG8 + 50 μL X0044 |
| | plasma |
| 12 | 95 μL PBS + 2.5 μL of 300 mg/ml PEG1 + 2.5 μL of 300 |
| | mg/ml + 50 μL X0044 plasma |
| 13 | 95 μL PBS + 1.67 μL of 300 mg/ml PEG3 + 1.67 μL of 300 |
| | mg/ml PEG4 + 1.67 μL of 300 mg/ml PEGS + 50 μL X0044 |
| | plasma |
| 14 | 95 μL PBS + 1.67 μL of 300 mg/ml PEG6 + 1.67 μL of 300 |
| | mg/ml PEG7 + 1.67 μL of 300 mg/ml PEG8 + 50 μL X0044 |
| | plasma |
| 15 | 95 μL PBS + 1.67 μL of 300 mg/ml PEG1 + 1.67 μL of 300 |
| | mg/ml PEG3 + 1.67 μL of 300 mg/ml PEG6 + 50 μLX0044 |
| | plasma |
| 16 | 95 μL PBS + 1.67 μL of 300 mg/ml PEG2 + 1.67 μL of 300 |
| | mg/ml PEG4 + 1.67 μL of 300 mg/ml PEG7 + 50 μL X0044 |
| | plasma |

TABLE 9
Plasma/PEG mixture protocol for each well of slide #2

| Well | Slide #2 Plasma/PEG Mixture Protocol |
|---|---|
| 1 | 100 μL PBS + 50 μL X0157 plasma |
| 2 | 95 μL PBS + 5 μL of 300 mg/ml PEG1 + 50 μL X0157 plasma |
| 3 | 100 μL PBS + 50 μL X0044 plasma |
| 4 | 95 μL PBS + 5 μL of 300 mg/ml PEG1 + 50 μL X0044 plasma |
| 5 | 100 μL PBS + 50 μL X0172 plasma |
| 6 | 95 μL PBS + 5 μL of 300 mg/ml PEG1 + 50 μL X0172 plasma |
| 7 | 100 μL PBS + 50 μL X0055 plasma |
| 8 | 95 μL PBS + 5 μL of 300 mg/ml PEG1 + 50 μL X0055 plasma |
| 9 | 100 μL PBS + 50 μL X0199 plasma |
| 10 | 95 μL PBS + 5 μL of 300 mg/ml PEG1 + 50 μL X0199 plasma |
| 11 | 100 μL PBS + 50 μL X0137 plasma |
| 12 | 95 μL PBS + 5 μL of 300 mg/ml PEG1 + 50 μL X0137 plasma |
| 13 | 95 μL PBS + 5 μL of 300 mg/ml PEG3 + 50 μL X0172 plasma |
| 14 | 95 μL PBS + 5 μL of 300 mg/ml PEG6 + 50 μL X0172 plasma |
| 15 | 95 μL PBS + 2.5 μL of 300 mg/ml PEG1 + 2.5 μL of 300 mg/ml |
| | PEG3 + 50 μL X0172 plasma |
| 16 | 150 μL PBS |

### c) Assay Protocol

To 100 μL of 15 mg/ml PEG in PBS or PBS with no PEG, 50 μL of plasma was added (for a 1:3 dilution of plasma and a 10 mg/ml total PEG concentration), and the mixture was incubated for 20 min at RT.

The slide to be assayed was assembled with a 16-well adapter. The pre-incubated plasma+PEG mixture was added to each well and incubated for 21 minutes at RT (no pre-assay wash).

The wells were aspirated using the vacuum system to remove the liquid.

To each well, 150 μL PBS-T was added. Wells were washed with PBS-T using the Biotek Washer (Program: ABWASH2, 3 Cycles). *Note: some residual PBS-T is always observed in the wells, typically approximately 50 μL.

To each well, 100 μL of Alexa 647-labeled antibody solution in PBS was added and incubated for 10 minutes at RT in the dark (covered). Wells were washed with PBS-T using the BiotTek Washer (Program: ABWASH2, 3 Cycles).

The slide was removed from the 16-cell adapter and rinsed further by immersing into 50 ml of PBS-T and inverting 10 times.

The PBS-T was decanted, and the slide was dried by centrifuging at 800 xg for 1 min two times).

The slide was scanned for fluorescence intensity using the GenePix 4300A (excitation 635 nm, PMT 600, 100% power).

The data were extracted using the GenePix software.

Specificity was calculated using the mean red fluorescence intensity (A signal divided by B signal or B signal divided by A signal). In this calculation, the fluorescence intensity was not background-subtracted.

III. Results From Slide #1

The experimental layout for slide #1 is shown in FIG. 21. Slide #1 is blank (Ep POEGMA slide WRD170-15) and has no spots. It contains X0044 plasma Type A pre-mixed with PEG, 10 mg/ml, which is known to produce a high substrate background with anti-hIgM. Alexa 647-labelled F(ab′)2 fragment goat anti-human IgM, Fc_{5μ} fragment specific, 1 μg/ml in PBS was used as the detection reagent.

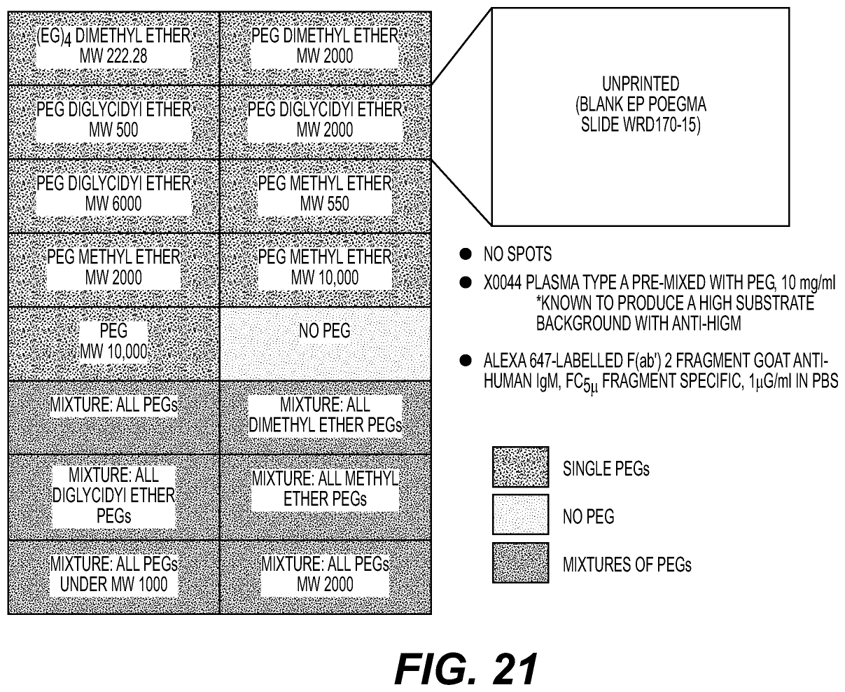

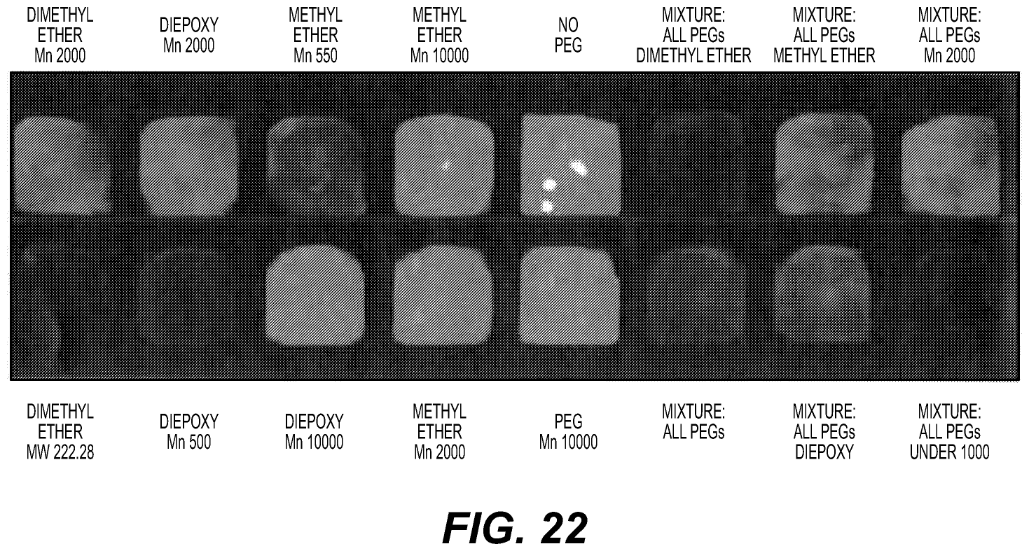

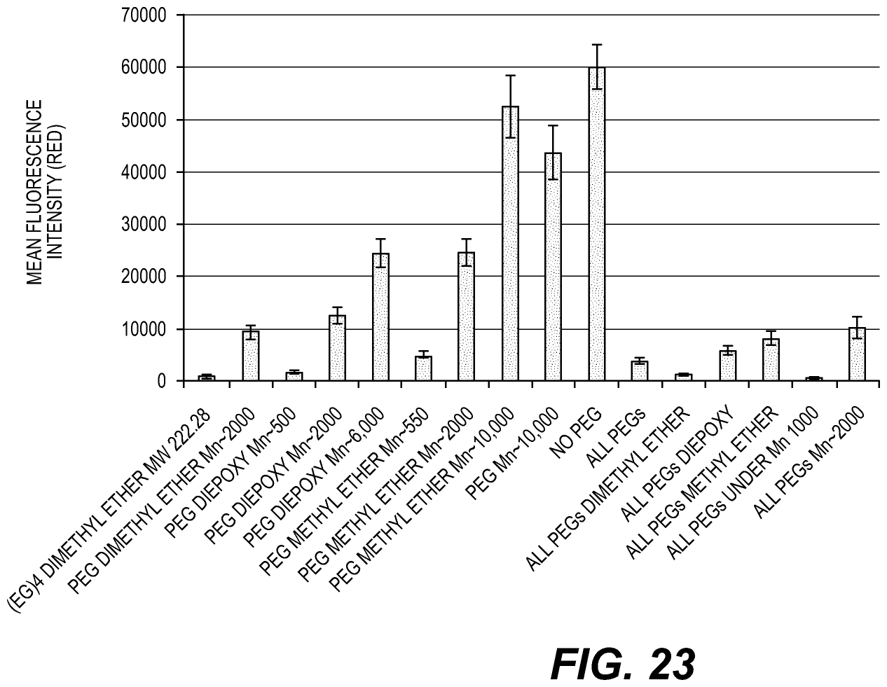

A significant reduction in background signal was observed when compared with the signal observed in the well with no PEG added to the plasma, with a trend in reduction based on the size of the PEG (small>medium>large) (FIG. 22). This was true of the dimethyl ether PEGs, the methyl ether PEGs, and the diglycidyl (or diepoxy) PEGs. The trend in background reduction based on end groups was tetraethylene glycol dimethyl ether>PEG diglycidyl ether Mn 500>PEG methyl ether Mn 550. Quantification of the data indicated a reduction of almost 2 orders of magnitude between the well in which no PEG was added and the well in which tetraethylene glycol dimethyl ether was added (Table 10 and FIG. 23).

TABLE 10
Average fluorescence intensity for each well

| | Mean Fluorescense | |
|---|---|---|
| PEG Added | Intensity (RED) | SD |
| (EG)4 dimethyl ether MW 22.28 | 672 | 153 |
| PEG dimethyl ether Mn 2000 | 9294 | 1271 |
| PEG diglycidyl ether (diepoxy) Mn 500 | 1674 | 199 |
| PEG diglycidyl ether (diepoxy) Mn 2000 | 12714 | 1456 |
| PEG diglycidyl ether (diepoxy) Mn 6,000 | 24416 | 2686 |
| PEG methyl ether Mn 550 | 5116 | 632 |
| PEG methyl ether Mn 2000 | 24707 | 2535 |
| PEG methyl ether Mn 10,000 | 52477 | 5874 |
| PEG Mn 10,000 | 43647 | 5102 |
| No PEG | 60039 | 4086 |
| all PEGs | 4066 | 536 |
| all PEGs dimethyl ether | 1352 | 66 |
| all PEGs diepoxy | 5920 | 858 |
| all PEGs methyl ether | 8300 | 1196 |
| all PEGs under Mn 1000 | 859 | 149 |
| all PEGs Mn 2000 | 10308 | 2042 |

IV. Results From Slide #2

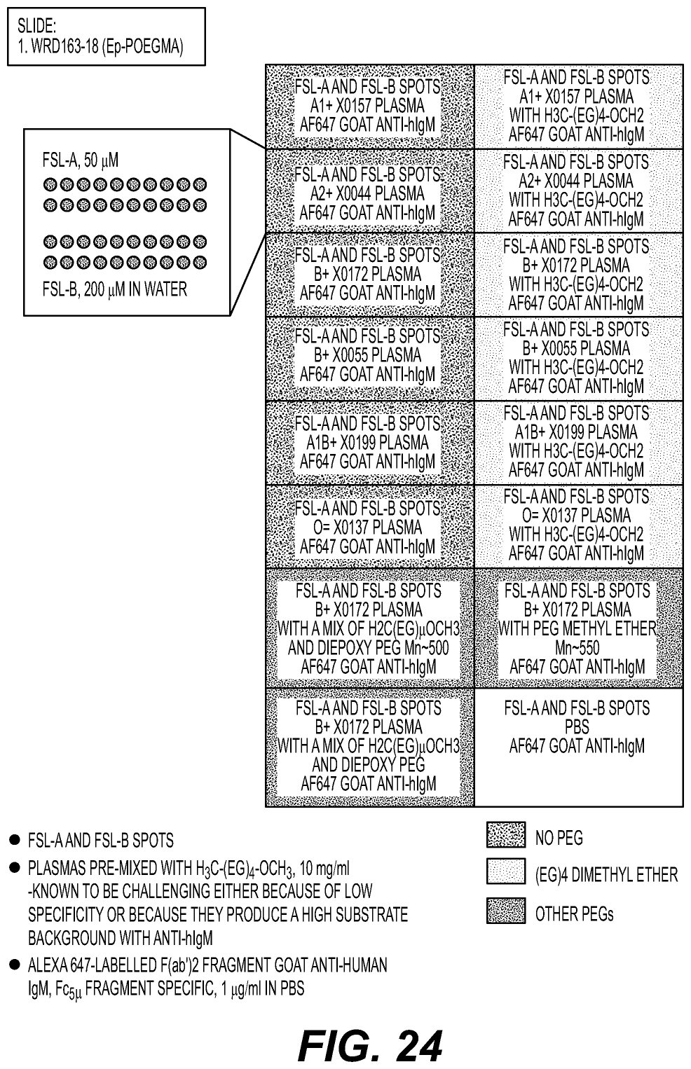

In order to determine if the experiment was reproducible with the same plasma, and if background reduction could be achieved with other plasmas, 6 plasma samples, including the plasma used in the first slide, were pre-incubated with tetraethylene glycol dimethyl ether. One of the plasmas (B+, R2r) was also pre-incubated with the other two small PEGs (PEG methyl ether Mn 550 and PEG diglycidyl ether Mn 500) and a mixture of tetraethylene glycol dimethyl ether and PEG diglycidyl ether Mn 500. One well was used as a control to confirm that no signal would be observed when no plasma was added to the well (PBS only) (FIG. 24).

In addition, in order to determine whether a reduction in background signal would allow observation of printed spots and their activity in an ABO reverse typing assay, slide #2 was printed with 2 rows of synthetic antigen A (FSL-A, 50 μM in water) and 2 rows of synthetic antigen B (FSL-B, 200 μM in water).

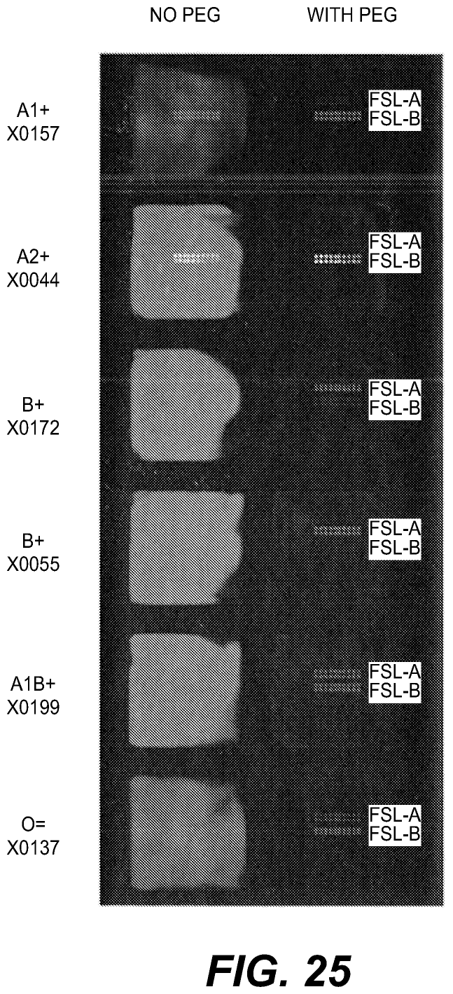

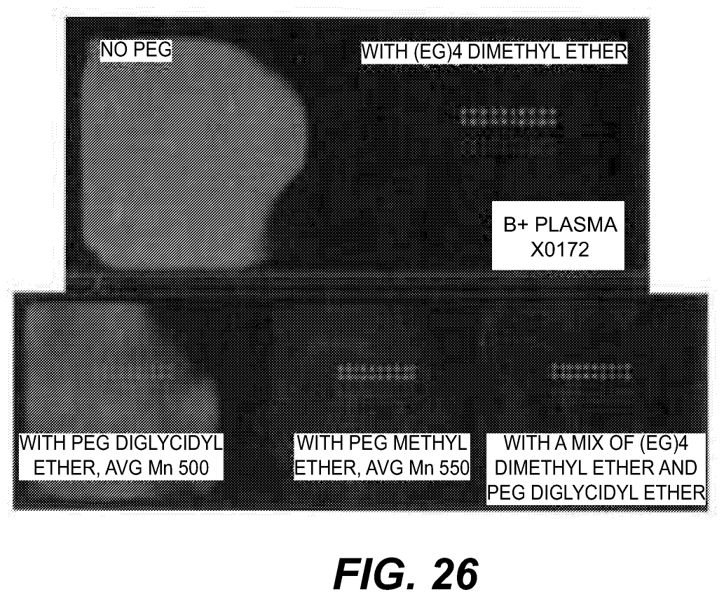

As shown in FIG. 25, a reduction in background signal was observed for all 6 of the plasmas tested, including the plasma tested on slide #1. Plasma (B+, R2r), which was dosed with all 3 small PEGs, showed the following trend in background reduction: tetraethylene glycol dimethyl ether>PEG methyl ether Mn 550>diglycidyl ether Mn 500 (FIG. 26). This observation was slightly different than the trend for plasma (A2+, R1R2).

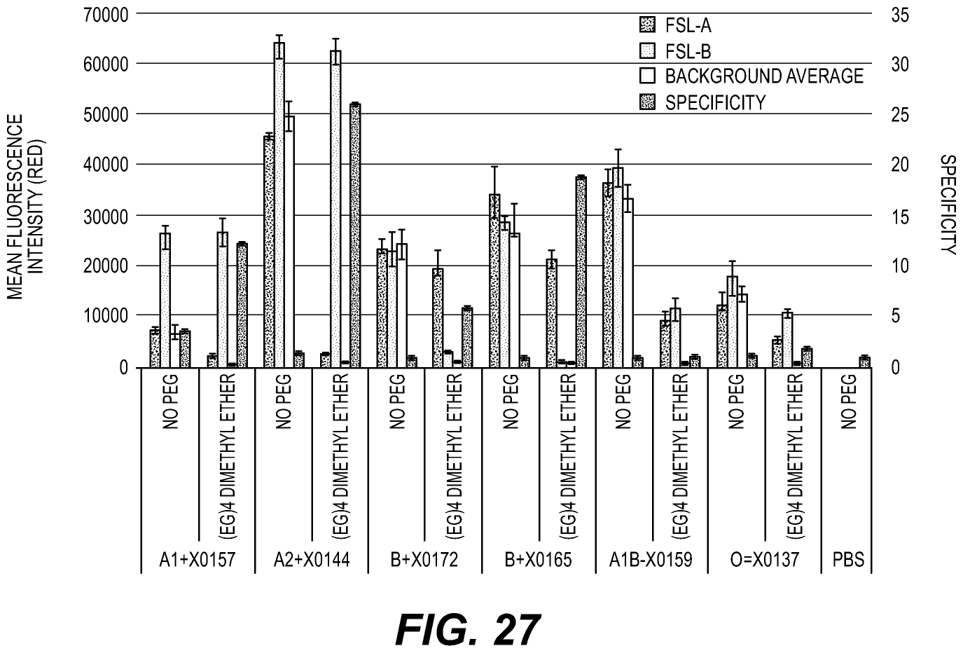

In 3 of the 6 plasmas, without pre-incubation with tetraethylene glycol dimethyl ether, the printed spots were completed washed out by the background signal (FIG. 6). The addition of PEG to the plasma resulted in the ability to visualize the spots for all of the plasmas. There was, however, some in-spot non-specific binding to the FSL spots exposed to AB plasma (a case where there should be no binding to either FSL). This has been observed for this plasma in past experiments using anti-hIgG as the secondary antibody as well, and requires more work to eliminate. Quantification of the results indicated that reduction of the background signal with PEG resulted in a decrease in the negative spots, which resulted in a higher specificity for the A and B plasmas (FIG. 27 and Table 11). Since there was no negative spot in printed FSLs exposed to 0 plasma (both FSL-A and FSL-B should fluoresce), specificity was calculated to be close to 2, with the signal from FSL-B being higher because of the higher printing concentration.

TABLE 11
Average Intensity and Specificity

| | | | Mean | | Mean | | Background | | |
|---|---|---|---|---|---|---|---|---|---|
| | | | Intensity | FSL-A | Intensity | FSL-B | Mean | Background | |
| Well | Plasma | PEG | (FSL-A) | SD | (FSL-B) | SD | Intensity | SD | Specificity |
| 1 | A1 + X0157 | No PEG | 7063 | 361 | 26487 | 1507 | 6550 | 779 | 3.75 |
| 2 | | (EG)4 dimethyl | 2160 | 324 | 26659 | 2340 | 376 | 46 | 12.34 |
| | | ether | | | | | | | |
| 3 | A2 + X0044 | No PEG | 45947 | 2985 | 64906 | 596 | 49652 | 2795 | 1.41 |
| | | (EG)4 dimethyl | 2405 | 275 | 62690 | 2815 | 889 | 158 | 26.07 |
| | | ether | | | | | | | |
| 5 | B + X0172 | No PEG | 23465 | 2006 | 23232 | 3224 | 24352 | 2627 | 1.01 |
| 6 | | (EG)4 dimethyl | 19252 | 3554 | 3196 | 169 | 726 | 84 | 6.02 |
| | | ether | | | | | | | |
| 13 | | Diepoxy PEG | 14188 | 1842 | 6754 | 642 | 8164 | 919 | 2.10 |
| 14 | | PEG methyl ether | 17411 | 1077 | 4054 | 404 | 2344 | 373 | 4.29 |
| 15 | | Mix: (EG)4 | 13175 | 1315 | 2848 | 229 | 674 | 103 | 4.63 |
| | | dimethyl ether | | | | | | | |
| | | and diepoxy PEG | | | | | | | |
| 7 | B + X0055 | No PEG | 34564 | 4833 | 28483 | 1436 | 29374 | 3068 | 1.21 |
| 8 | | (EG)4 dimethyl | 21500 | 1829 | 1138 | 120 | 903 | 111 | 18.90 |
| | | ether | | | | | | | |
| 9 | A1B + X0199 | No PEG | 36701 | 2600 | 39426 | 3669 | 33437 | 2951 | 1.07 |
| 10 | | (EG)4 dimethyl | | | | | | | |
| | | ether | 9463 | 707 | 11997 | 1372 | 731 | 93 | 1.27 |
| 11 | O = X0137 | No PEG | 12439 | 2545 | 17744 | 3197 | 14572 | 1710 | 1.43 |
| 12 | | (EG)4 dimethyl | 5438 | 475 | 10748 | 1051 | 336 | 49 | 1.98 |
| | | ether | | | | | | | |
| 16 | PBS | No PEG | 77 | 4 | 70 | 4 | 63 | 4 | 1.10 |

V. Discussion

The addition of small molecular weight PEGs, especially tetraethylene glycol dimethyl ether, drastically reduced the background signal from Type A plasma when using Alexa 647-labeled anti-hIgM as a secondary antibody. The reduction was dependent on the size of the PEG. The experiment can be repeated with equimolar concentrations of PEG instead of mass % concentrations, although care must be taken to avoid protein precipitation from the plasma at high concentrations of higher molecular weight PEGs (no precipitation was observed at 10 mg/ml, used in the current experiment).

### Example 11: The Cell Double-Antigen Bridge Assay in an ABO Reverse Typing Assay

The study was carried out to determine whether a cell double antigen bridge assay, in which cells are used as both the capture and detection reagents, would work in an ABO reverse typing assay. Using FSL-antigens as the capture reagent and cells for the detection reagent was also compared.

The printed FSL-antigens are effective capture molecules when using an anti-hIgG secondary antibody for detection. In this experiment, a double antigen bridge assay was performed with cells as the detection reagents.

Experimental Design/Layout: Fresh ABO cells (non-fixed), ghost ABO cells, and FSL-A and -B were printed on an ePOEGMA slide. Plasma was mixed with PEG for a 3× dilution of plasma and 100 mM total PEG concentration. The wells in the right column were washed after plasma exposure, the wells in the left column were not washed. A mixed suspension of reference A and B cells (1-2% hematocrit) was used for detection. After incubation with the cells, the slide was turned on its side so the unbound cells could settle onto the side of the wells and an image of the spots could be taken.

Information about the print slides, printed cells, other print reagents, detection reagents, other reagents, plasmas, and equipment used in the studies are provided in Table 12.

TABLE 12
Materials, Reagents, and Equipment
Print slides

| Slide/ | Lot Number | | | |
|---|---|---|---|---|
| Barcode | (ePOEGMA) | Thickness | Printing Date | Array Details |
| WRD125-7/ | 125 | 18.3 nm | 17 March 2017 | FSL-antigens; FSL-NHAc; non-fixed |
| 10134233 | | | | cells (10%); ghost cells (15%) |

Printed cells

Printed on
the following

| Donor | slides | Anticoagulant | ABO | Rh |
|---|---|---|---|---|
| X0202 | WRD125-7 | EDTA | A1= | rr |
| X0081 | WRD125-7 | EDTA | B= | rr |
| X0138 | WRD125-7 | EDTA | O= | rr |
| X0202 | WRD125-7 | EDTA | A1= | rr |
| X0081 | WRD125-7 | EDTA | B= | rr |
| X0138 | WRD125-7 | EDTA | O= | rr |

Catalog

| | | |
|---|---|---|
| Reagent | Manufacturer | Number |

Other print reagents

| | | |
|---|---|---|
| FSL-tetra A | KODE | N/A |
| FSL-tetra B | KODE | N/A |

Detection Reagents

| Reagent Red | Immucor | 2345 |
|---|---|---|
| Blood Cells | | |

Other Reagents

| | | |
|---|---|---|
| Poly(ethylene | Aldrich | 445886-50ML |
| glycol) | | |
| dimethyl | | |
| ether avg Mn | | |
| 500 | | |
| Poly(ethylene | Aldrich | 475696-100ML |
| glycol) | | |
| diglycidyl | | |
| ether avg Mn | | |
| 500 | | |
| pHix | Immucor | 5070 |
| phosphate | | |
| buffer, 200 | | |
| ml | | |
| Blood Bank | Immucor | 23-062-125 |
| Saline, | | |
| Isotonic | | |
| Solution | | |
| 0.90% w/v, | | |
| 10 L | | |
| Tween 20 | Sigma | P9416-100ML |
| Red Cell | Immucor | 5036 |
| Diluent | | |
| (RCD), Red | | |
| Blood Cell | | |
| Storage | | |
| Solution | | |
| Digitonin | Sigma | D141 |

Plasmas

Donor

| (Plasma) | Anticoagulant | ABO | Rh |
|---|---|---|---|
| X0044 | EDTA | A2+ | R1R2 |
| X0011 | EDTA | A1+ | R1R1 |
| X0080 | EDTA | B= | rr |
| X0068 | EDTA | B+ | R2r |
| X0049 | EDTA | A2Bw+ | R1wR1 |
| X0160 | EDTA | A2B+ | Ror |
| X0112 | EDTA | O+ | R1R2 |
| X0214 | EDTA | O+ | R1R1 |

Buffer, PEG, and Detection Reagent were prepared as described herein. To prepare PBS, 100 ml of pHix phosphate buffer was added to 10 L of blood bank saline. To prepare PBS/0.1% Tween 20 (PBS-T), 1 ml of Tween 20 was added to 1 L of PBS.

To prepare the detection reagent, 1 ml of reagent A₁ cells and 1 ml of reagent B cells were added to 2 ml of PBS (1-2% hematocrit). PEG stock solution was prepared by dissolving PEG dimethyl ether (Mₙ 500) and PEG diglycidyl ether (Mₙ 500), each at a concentration of 75 mM, for a total PEG concentration of 150 mM. The protocol for preparing the PEG stock solution is described in Table 13.

TABLE 13
Protocol for making the PEG stock solution

| | | | | | Volume needed |
|---|---|---|---|---|---|
| | | | Molecular | | to make 10 ml of |
| | Total PEG | | Weight | Density | PEG solution in |
| Mixture | concentration | Reagent | (g/mol) | (g/ml) | PBS |
| 1:1 PEG dimethyl | 150 mM | PEG dimethyl | 500 | 1.05 | 0.375 ml |
| ether Mn 500/PEG | | ether | | | |
| diglycidyl ether Mn | | PEG diglycidyl | 500 | 1.14 | 0.329 ml |
| | | ether | | | |
| | | PBS | N/A | N/A | 9.296 ml |

Sample Processing follows the procedures provided below. Plasma was obtained from donors. Donor Type A, B, AB, and O whole blood was centrifuged at 3000×g for 10 min to separate the plasma from the red blood cells; plasma was aliquoted and stored in the freezer; buffy coat was discarded and packed cells were washed 5 times with red cell diluent (RCD) before being resuspended in RCD and stored in the refrigerator. Synthetic antigens FSL-A and FSL-B were thawed and diluted to 10 μM or 50 μM in water for printing. Frozen plasma was thawed and used the same day, or stored at 4° C. until further use.

Cells were prepared as following: Ghost cells lysed with digitonin (labelled digi): 1.5 ml of 3% hematocrit cells in RCD were washed twice with PBS, then resuspended to 1.5 ml in PBS; 50 μL of 5 mg/ml digitonin in PBS was added to lyse the cells (161 μg/ml total digitonin concentration), which were immediately centrifuged at 3000×g at 4° C. for 40 min; the red supernatant was removed, and the ghost cells were washed with PBS 5 times, centrifuging at 3000×g for 5 min at RT, before being resuspended in 100 μL of PBS (15% by volume, used for printing).

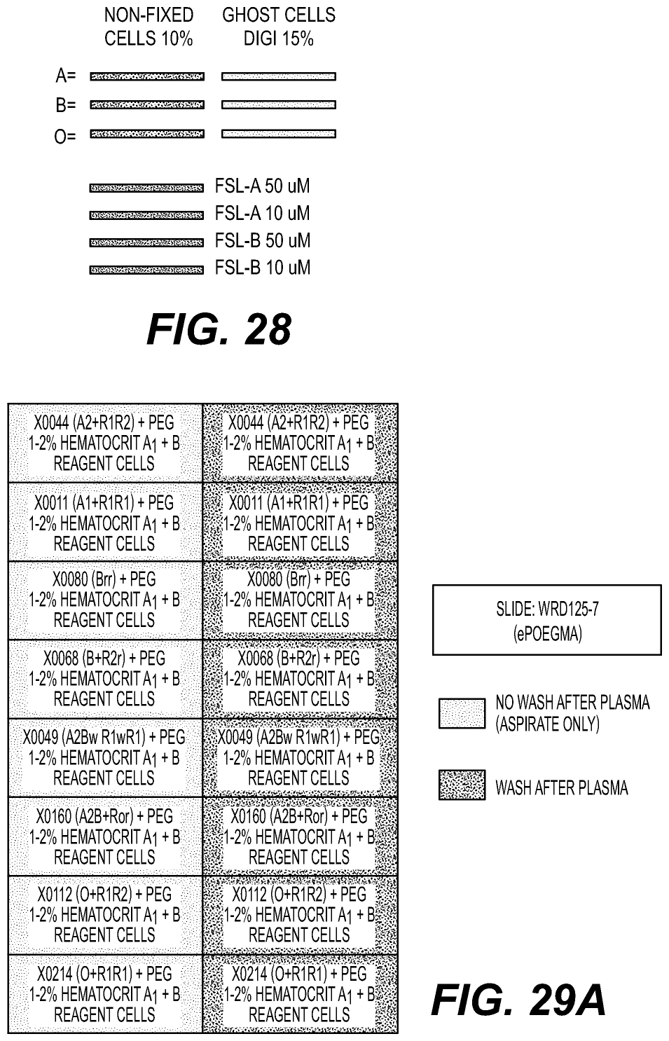

Fresh, non-fixed cells: 150 ul of 3% hematocrit cells in RCD were washed twice with PBS, then resuspended to 45 μL in PBS for printing (10% by volume). The print layout is shown in FIG. 28.

### Assay Protocol

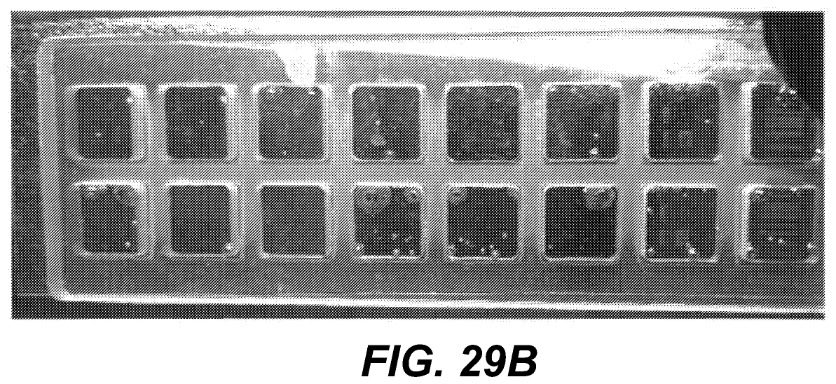

An exemplary assay protocol is provided below.
1. The slide to be assayed was assembled with the 16-well FlexWell adapter.
2. 50 μL of plasma was added to 100 μL of 150 mM PEG in PBS, (for a 1:3 dilution of plasma and a 100 mM total PEG concentration), pre-mixed, added to the well according to the layout shown in FIG. 29A, and incubated for 20 minutes at RT on a shaker (no pre-assay wash).
3. In the right hand column: The wells were aspirated using a vacuum aspirator to remove the liquid. To each well, 150 μL PBS-T was added. Wells were washed with PBS-T using the Biotek Washer (Program 28: FLEXWELL MSM, 3 Cycles). *Note: some residual PBS-T is always observed in the wells, but the exact volume for this program is unknown.
4. All wells in both the left-hand column (containing plasma) and right-hand column (containing PBS-T) were aspirated using a vacuum aspirator to remove the liquid.
5. Immediately, 165 μL of a 1:1 mixture of A₁ and B reagent cells at a total of 1-2% hematocrit (0.5-1% each) was added to each well.
6. The wells were sealed with transparent tape, and the slide was incubated for 10 minutes at RT on a shaker.
7. The slide was turned on its side to allow the unbound reagent red cells to settle to the sides of the wells by gravity (see picture in FIG. 29B).
8. Images of the wells were taken using the Scienion printer camera, the microscope, and the inverted microscope.

### Results and Discussion

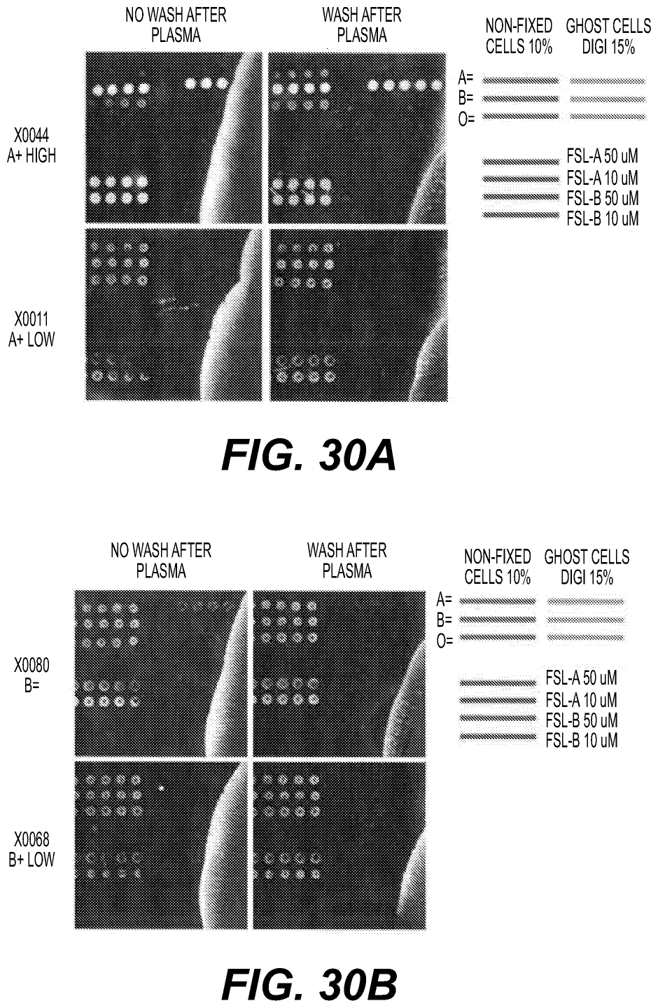

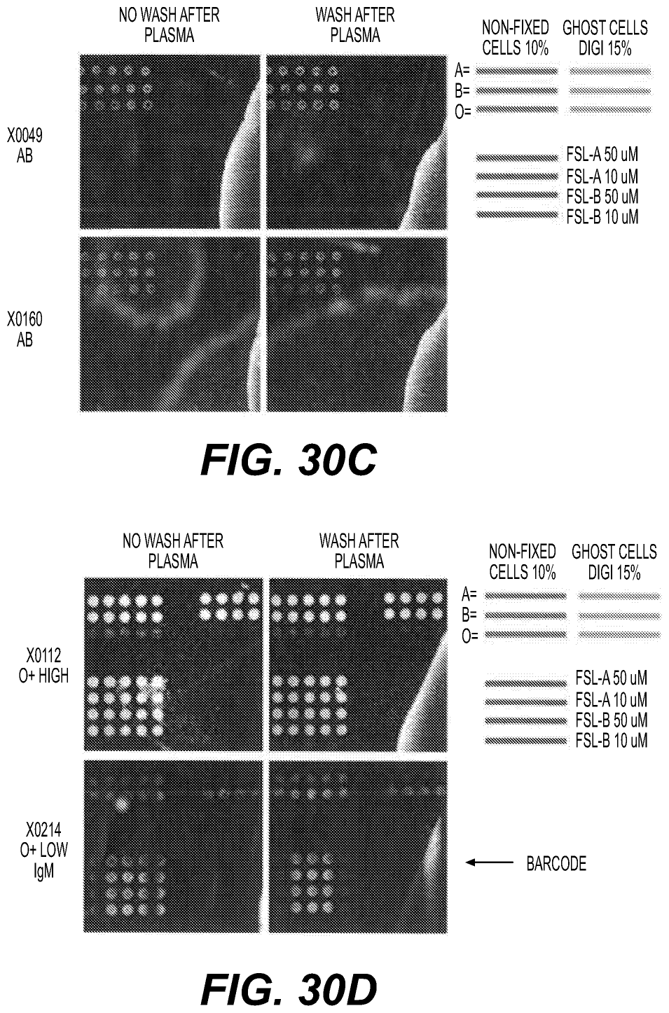

After exposure to plasma and then reagent A and B cells, the slide was turned on its side to allow the unbound cells to settle with gravity. Once the cells settled (after about an hour), the Scienion printer camera was used to take an image of the slide, and that image is available in the shared drive. The slide was then repositioned on its side overnight, and the inverted microscope was used to take darkfield images the following day. The darkfield images are shown in FIGS. 30A-30D.

FIG. 30A shows exemplary darkfield microscopy images of wells that were exposed to one high titer A donor and one low titer A donor. Washing after aspirating the plasma did not seem to make a difference. Binding was observed on the FSL-B spots for both high and low titer plasmas. Binding to the B=non-fixed and ghost cells was observed for the high titer plasma only (X0044). No non-specific binding was observed. Note that the printed non-fixed cells can be seen in the image, but the positive spots can be differentiated from the negative spots for the high titer plasma. The particles spreading from the bottom right corner towards the array in each well are the settled red blood cells which then began to shift once the slide was turned from its side to its back in order to take the image. FIG. 30B shows exemplary darkfield microscopy images of wells that were exposed to one medium titer B donor and one low titer B donor. Washing after aspirating the plasma seemed to result in lower binding efficiency. Binding was observed on the FSL-A spots for both plasmas. Binding to the A=non-fixed and ghost cells was observed for the medium titer plasma only, and the binding was somewhat faint. The faint binding observed in these images could be, in part, attributed to the images being taken after a full night of settling. In the images taken with the Scienion printer camera (available on the shared drive), a larger amount of binding to the A=cells exposed to X0080 was observed, and no difference was observed between washing and no washing after exposure to plasma. No non-specific binding was observed. FIG. 30C shows exemplary darkfield microscopy images of wells that were exposed to two AB donors. Washing after aspirating the plasma did not seem to make a difference. No non-specific binding was observed. Note that the printed non-fixed cells can be seen in the image. FIG. 30D shows exemplary darkfield microscopy images of wells that were exposed to one high titer O donor and one low hIgM titer O donor. Binding was observed on the FSL-antigens, the non-fixed cells, and the ghost cells, but binding was weaker and asymmetric for the low hIgM titer O plasma (X0214). Washing after aspirating the plasma did not make a huge difference, but there appeared to be a small amount of non-specific binding to the O=cells exposed to the high titer O donor (X0112), perhaps a little more in the washed well. Note that the printed non-fixed cells can be seen in the image, but the positive spots can be differentiated from the negative spots.

In general, the wells that were washed after exposure to plasma behaved the same as the wells that were not washed. The only exception was the well exposed to X0112, a high titer O donor, which exhibited a small amount of non-specific binding to the printed O=cells when the well was washed after exposure to plasma (compared to the unwashed well, which exhibited even less non-specific binding to the O=cells). These two wells were the only wells where non-specific binding was observed.

For the wells exposed to A donors, binding was observed on the FSL-B spots for both high and low titer plasmas. Binding to the printed B=cells (non-fixed and ghost) was observed for the high titer plasma only (X0044). No non-specific binding was observed. Note that the printed non-fixed cells can be seen in the image, but the positive spots can be differentiated from the negative spots for the high titer plasma.

For the wells exposed to B donors, binding was observed on the FSL-A spots for both the medium and low titer plasmas. There was some binding to the printed A=cells exposed to the medium titer B plasma, but it was a bit weak, and weaker still for the washed well. However, binding was stronger and equal between the washed and unwashed wells in the image taken with the Scienion printer camera the day of the experiment before the cells were allowed to settle overnight (image not shown here, but available in the shared drive). The full night of settling may have allowed gravity to pull the cells that were specifically bound, but weakly bound, from the spots.

For wells exposed to the AB donors, where no binding should occur to any of the printed spots, there was no non-specific binding observed. For wells exposed to the O donors, binding was observed on all of the printed A and B non-fixed and ghost cells, as well as the FSL-antigens. The binding was weaker and asymmetric for the low hIgM titer O plasma (X0214). Washing after aspirating the plasma did not make a huge difference, but there appeared to be a small amount of non-specific binding to the O=cells exposed to the high titer O donor (X0112), perhaps a little more in the washed well.

Lastly, the concentration of the printed FSL-antigen spots appeared to have an effect, with higher binding observed on the 10 pM spots compared to the 50 pM spots. Another experiment is needed to determine the optimum FSL-antigen print concentration for the double antigen assay using cells as the detection reagent.

### Conclusions

The double antigen bridge assay using FSL-antigens as the capture reagent and cells as the detection reagent worked for all of the plasmas tested.

The cell double antigen bridge assay using cells as both the capture and detection reagents worked with the higher titer A and B plasmas, both AB plasmas, and both 0 plasmas, while using FSL-antigens as the capture reagent and cells as the detection reagent worked with both low and high titer A and B plasmas. In this experiment, the printed FSL-antigens are a better capture reagent than the cells.

There was little to no difference between the wells that were washed after exposure to plasma and wells that were not washed.

### Example 12: Reverse Typing of 53 Plasma Donors Using Gravity Double Antigen Cell Bridge Assay

This example tests the performance of the gravity double antigen cell bridge assay with 53 plasma samples. In this ABO reverse typing format, FSL-antigens are used as the capture reagent and antigen positive red blood cells are used as the detection reagent.

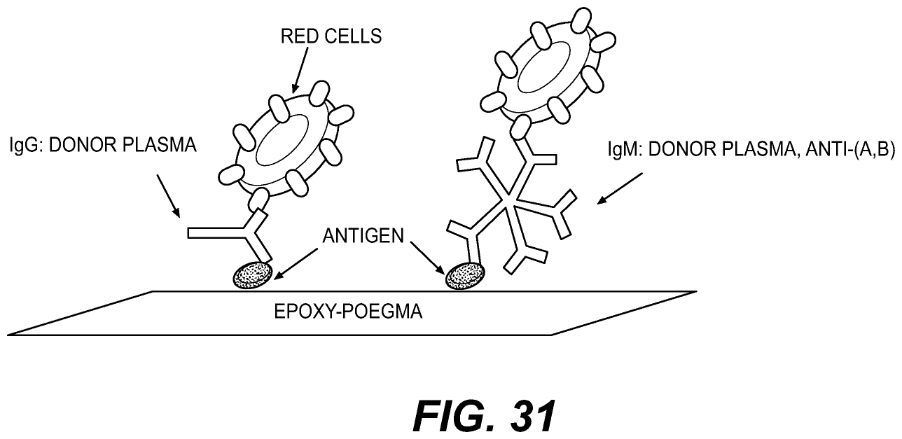

The double antigen cell bridge assay, with FSL-antigens as the capture reagent and a mixture of reference A and B cells as the detection reagent (illustrated in FIG. 31), was used successfully to type A, B, AB, and O plasmas, with no undesired binding observed (shown in Example 11). Better binding was observed on FSL-antigen spots printed at 10 μM and 17 compared to those printed at 50 pM (Example 11). The slides were imaged with a light microscope using either transmitted light (4×) or dark-field (20×), and the results were determined visually.

In this experiment, a larger pool of donors was tested to determine whether this assay format performed as well as our best practice ABO reverse typing assay, which uses a fluorescently-labeled secondary antibody as the detection reagent. In the cell bridge assay, since separation of the unbound cells takes place via gravity when the slide is turned on its side (illustrated in FIG. 29B), imaging with a microscope is difficult because the settled cells migrate back towards the array when the slide is turned back and placed flat under the microscope. Here, a new imaging setup was tested. This imaging setup, built in-house, allows the slide to be imaged while on its side. A light is shined through a slit towards the slide at an angle, and a camera captures the light scattered from the cells (FIG. 32A). Alternatively, two light beams can be used for imaging. An exemplary imaging system using two light beams is illustrated in FIG. 32B.

Experimental Design/Layout: FSL-A and FSL-B were printed on ePOEGMA slides at concentrations of 15 μM and 50 μM. A fiducial consisting of 33F1 anti-RBC spots was also printed to mark the orientation of the array. Plasma diluted 1:3 in PBS was incubated for 20 min, after which the wells were washed with PBS-T. A mixed suspension of reference A and B cells was used for detection. After incubation with the cells, the slide was sealed with tape and turned on its side so the unbound cells could settle overnight onto the side of the wells and an image of the spots could be taken with the new imaging setup. The captured images, taken with a gain of 1.0 and an exposure time of 30 s, were converted from bitmap files to 16-bit tif files in ImageJ and opened in the GenePix software to extract the data. Since the captured images were 8-bit, the extracted values were multiplied by 256 to place them on a scale similar to the data normally extracted from a GenePix scan. The slides were also imaged with the upright microscope using transmitted light and a 4× objective as a backup to check any anomalies in the data analysis.

The selection of donors for this experiment was based on the results of the data analysis from a 300-sample study using the traditional ABO reverse typing assay with a fluorescently labeled anti-hIgM for detection. Overall, that assay performed well. For some donors, the signal of the negative spot was above the chosen cutoff value of 3000, indicating high non-specific binding or cross-reactivity. For some donors, the positive signal was below the cutoff value of 3000, which in many cases could be attributed to the low titer of anti-A or anti-B present in that particular plasma. However, some Type O donors that are considered to be medium or high titer plasmas also exhibited low positive signal. A possible explanation could be a difference in affinity for the printed FSL-antigens between antibodies that are IgG in nature and those that are IgM. If most of the expected antibodies are IgG, and if those IgG antibodies have a higher affinity for the FSL-antigens than the IgM antibodies, then the IgGs could out-compete the IgMs. When the captured antibodies are detected with an anti-hIgM secondary antibody, as was the case in the 300-sample study, then the captured IgGs would not be detected. With these results in mind, the donors selected for this experiment were chosen for one of the following reasons:

1. Low titer donors

2. Donors exhibiting high non-specific binding or cross-reactivity

3. Type O donors with high IgG (anti-A or anti-B that is IgG in nature)

4. Type O donors with low IgG (anti-A or anti-B that is mostly IgM in nature)

5. A selection of medium and high titer donors in order to provide a good range

A table of the donors tested, with the results of the titration assays run on Immucor's Neo, is shown in Table 18. Donors that exhibited a positive signal below the cutoff in the 300-sample study are subject numbers 5, 7-9, 11, 12, 14, 22, 23, 30, 42, 49, 51, and 53 in Table 18; donors that exhibited a negative signal above the cutoff correspond to subject numbers 1, 3, 29, and 38 in Table 18. For Type AB donors, a negative signal above the cutoff for both FSL-A and FSL-B are subject numbers. 33, 35, and 37 in Table 18. For Type O donors, a positive signal below the cutoff for both FSL-A and FSL-B correspond to subject numbers 44-46 and 48. There were 15 Type A, 15 Type B, 8 Type AB, and 15 Type O donors, totaling 53. Donor X0044, a high titer A donor, was run on the first well of all four slides to determine the reproducibility of the assay from slide to slide.

Materials, reagents, and Equipment used in this experiment are provided in Tables 14-19 below.

TABLE 14
Print slides

| | Slide/ | | |
|---|---|---|---|
| | Barcode | Thickness | Array Details |
| 1 | WRD261-1/ | 28.1 nm | FSL-A and FSL-B, printed at 15 and 50 μM; |
| | 10171998 | | 33F1 anti-RBC; baked at 60° C. overnight, |
| | | | then stored in vacuum desiccator for 4 weeks |
| 2 | WRD261-7/ | 28.1 nm | FSL-A and FSL-B, printed at 15 and 50 μM; |
| | 10171992 | | 33F1 anti-RBC; baked at 60° C. overnight, |
| | | | then stored in vacuum desiccator for 4 weeks |
| 3 | WRD261-11/ | 28.1 nm | FSL-A and FSL-B, printed at 15 and 50 μM; |
| | 101671986 | | 33F1 anti-RBC; baked at 60° C. overnight, |
| | | | then stored in vacuum desiccator for 4 weeks |
| 4 | WRD261-19/ | 28.1 nm | FSL-A and FSL-B, printed at 15 and 50 μM; |
| | 10171977 | | 33F1 anti-RBC; baked at 60° C. overnight, |
| | | | then stored in vacuum desiccator for 4 weeks |

TABLE 15
Print reagents

| | | | Catalog |
|---|---|---|---|
| | Reagent | Manufacturer | Number |
| 1 | FSL-tetra A | KODE | N/A |
| | 15 μM and 50 μM | | |
| | in water | | |
| 2 | FSL-tetra B | KODE | N/A |
| | 15 μM and 50 μM | | |
| | in water | | |
| 3 | 33F1 (anti-RBC) | Immucor | N/A |
| | concentrated on | | |
| | 20 Jun 2017, 0.25 | | |
| | mg/ml in PBS | | |

TABLE 16
Detection Reagents

| | | | Catalog |
|---|---|---|---|
| | Reagent | Manufacturer | Number |
| 1 | Reagent Red Blood | Immucor | 0002345 |
| | Cells (2-4% | | |
| | suspension); diluted | | |
| | to 1-2% in PBS for | | |
| | the assay | | |

TABLE 17
Other Reagents

| | | | Catalog |
|---|---|---|---|
| | Reagent | Manufacturer | Number |
| 1 | pHix phosphate | Immucor | 0005070 |
| | buffer, 200 ml | | |
| 2 | Blood Bank | Thermo | 23-062-125 |
| | Saline, Isotonic | Scientific | |
| | Solution 0.90% | | |
| | w/v, 10 L | | |
| 3 | Tween 20 | Sigma | P9416-100ML |

TABLE 18
Plasmas

| | | | IgM anti-A | IgM anti-B | IgG anti-A | IgG anti-B |
|---|---|---|---|---|---|---|
| | Donor | ABO | titer | titer | titer | titer |
| 1 | R0121 | A | N/A | 64 | N/A | = |
| 2 | R0163 | A | N/A | 2 | N/A | = |
| 3 | R0251 | A | N/A | 64 | N/A | 128 |
| 4 | R500217 | A | N/A | 16 | N/A | 16 |
| 5 | W0020 | A | N/A | 1. | N/A | = |
| 6 | W0056 | A | N/A | 1 | N/A | 1 |
| 7 | W0112 | A | N/A | 8 | N/A | 1 |
| 8 | W0137 | A | N/A | 2 | N/A | 8 |
| 9 | W0143 | A | N/A | 4 | N/A | 8 |
| 10 | W0252 | A | N/A | 16 | N/A | = |
| 11 | X0007 | A | N/A | 4 | N/A | = |
| 12 | X0018 | A | N/A | 4 | N/A | = |
| 13 | X0037 | A | N/A | 8 | N/A | 64 |
| 14 | X0041 | A | N/A | 4 | N/A | 8 |
| 15 | X0044* | A | N/A | 64 | N/A | 8 |
| 16 | R0298 | B | 64 | N/A | 8 | N/A |
| 17 | R0526 | B | 32 | N/A | = | N/A |
| 18 | R329943 | B | 1 | N/A | 4 | N/A |
| 19 | W0101 | B | 32 | N/A | 32 | N/A |
| 20 | W0103 | B | 16 | N/A | 64 | N/A |
| 21 | W0109 | B | 2 | N/A | 8 | N/A |
| 22 | X0060 | B | 4 | N/A | 4 | N/A |
| 23 | X0062 | B | 8 | N/A | 8 | N/A |
| 24 | X0064 | B | 16 | N/A | 16 | N/A |
| 25 | X0070 | B | 64 | N/A | = | N/A |
| 26 | X0071 | B | 128 | N/A | 16 | N/A |
| 27 | X0080 | B | 8 | N/A | 64 | N/A |
| 28 | X0154 | B | 8 | N/A | = | N/A |
| 29 | X0185 | B | 16 | N/A | 8 | N/A |
| 30 | X0191 | B | 4 | N/A | 4 | N/A |
| 31 | R0166 | AB | N/A | N/A | N/A | N/A |
| 32 | R0237 | AB | N/A | N/A | N/A | N/A |
| 33 | R0311 | AB. | N/A | N/A | N/A | N/A |
| 34 | R328408 | AB | N/A | N/A | N/A | N/A |
| 35 | R329868 | AB | N/A | N/A | N/A | N/A |
| 36 | W0023 | AB | N/A | N/A | N/A | N/A |
| 37 | W0139 | AB | N/A | N/A | N/A | N/A |
| 38 | X0160 | AB | N/A | N/A | N/A | N/A |
| 39 | R0178 | 0 | 16 | 16 | 128 | 128 |
| 40 | R0215 | 0 | 128 | 8 | 128 | 128 |
| 41 | R0276 | 0 | 32 | 32 | 2 | 2 |
| 42 | R0283 | 0 | 4 | 4 | 128 | 128 |
| 43 | R314404 | 0 | 8 | 32 | 128 | 128 |
| 44 | W0099 | 0 | 2 | 1 | 1 | 4 |
| 45 | W0116 | 0 | 4 | 2 | 128 | 128 |
| 46 | W0145 | 0 | 2 | 4 | 1 | 64 |
| 47 | W0185 | 0 | 64 | 8 | 128 | 64 |
| 48 | X0082 | 0 | 4 | 2 | 1 | 2 |
| 49 | X0091 | 0 | 2 | 32 | 4 | 32 |
| 50 | X0098 | 0 | 32 | 8 | 128 | 2 |
| 51 | X0141 | 0 | 128 | 2 | 32 | 2 |
| 52 | X0143 | 0 | 8 | 128 | 64 | 128 |
| 53 | X0193 | 0 | 1 | 4 | = | 32 |

I. Protocols

Buffer/PEG/Detection Reagent Preparation: To prepare PBS, 100 ml of pHix phosphate buffer was added to 10 L of blood bank saline. To prepare PBS/0.1% Tween 20 (PBS-T), 1 ml of Tween 20 was added to 1 L of PBS. To prepare the detection reagent, 1 ml of reagent A1 cells and 1 ml of reagent B cells were added to 2 ml of PBS (1-2% hematocrit).

Sample Processing: Donor plasma was obtained. Donor Type A, B, AB, and O whole blood was centrifuged at 1000×g for 10 min to separate the plasma from the red blood cells; plasma was aliquoted and stored in the freezer for long term storage. Frozen plasma was thawed and used the same day, or stored at 4° C. until further use.

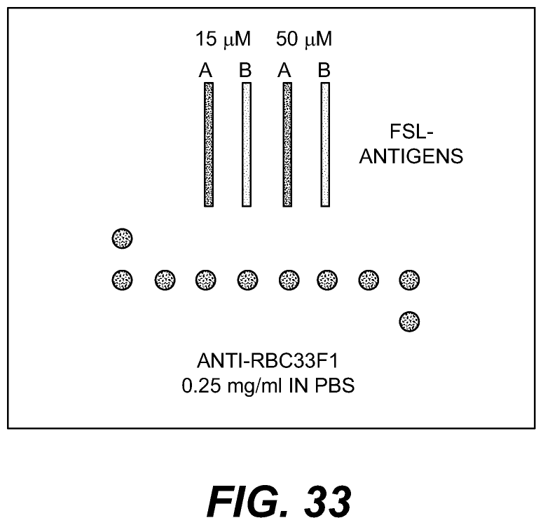

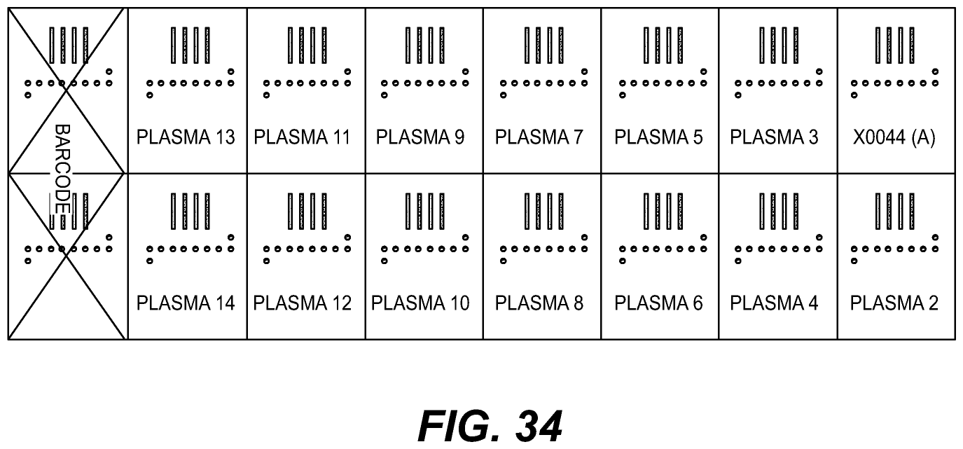

Synthetic antigens FSL-A and FSL-B were thawed and diluted to 15 or 50 μM in water for printing (the print layout is shown in FIGS. 33 and 34). Concentrated 33F1 was diluted to 0.25 mg/ml in PBS for printing.

Assay Protocol (4 Slides, Performed One Slide at a Time):

The slide to be assayed was assembled with the 16-well FlexWell adapter. Diluted plasma (50 μl plasma+100 μl PBS) was added to each well according to the layout in the attached supporting documents and incubated for 20 minutes at RT. The wells were aspirated using a vacuum aspirator to remove the liquid. To each well, 100 μl PBS-T was added. Wells were washed with PBS-T using the Biotek Washer (Program 28: FLEXWELL MSM, 3 Cycles). Some residual PBS-T is always observed in the wells, but the exact volume for this program is unknown. The residual liquid was aspirated from the wells using a vacuum aspirator and immediately replaced with 170 μl of a 1:1 mixture of A1 and B reagent cells at a total of 1-2% hematocrit (0.5-1% each). This step was performed one well at a time. The wells were sealed with transparent tape, and the slide was incubated for 10 minutes at RT.

The slide was turned on its side overnight to allow the unbound reagent red cells to settle to the sides of the wells by gravity. Scattering images of 8 wells at a time were taken using the imaging setup the following day (LuCam software, gain of 1.0, exposure time of 30 s). Due to overfilling in an attempt to avoid bubbles, some of the wells leaked overnight. In some cases, leaky wells leaked cells into the well below, obstructing the view of the binding of detection cells on the array. After the initial images were captured, the tape was removed and replaced on each slide. Some PBS was added to make up for the lost volume in order to avoid bubbles. The slides were turned on their side again to allow the resuspended cells to settle once again, and additional images were taken. For wells that were obstructed by leaky wells located above them, the new images were used for data analysis.

Microscope images were then taken (transmitted light, 4× objective). In some wells, the liquid disturbance during the tape change stripped the bound cells off of the array, but most of the wells were unaffected. The 8-bit BMP images were first opened in Image J in order to convert them to 16-bit TIF files. The pixel data, however, was still 8-bit (0-255 intensity range). The 16-bit TIF files were then opened in the GenePix software, which was used to extract the background-subtracted mean and median scattering intensity data. The extracted values were multiplied by 256 to place them on a 16-bit scale (0-65, 535 intensity range) similar to the data normally extracted from a GenePix scan.

II. Results and Discussion

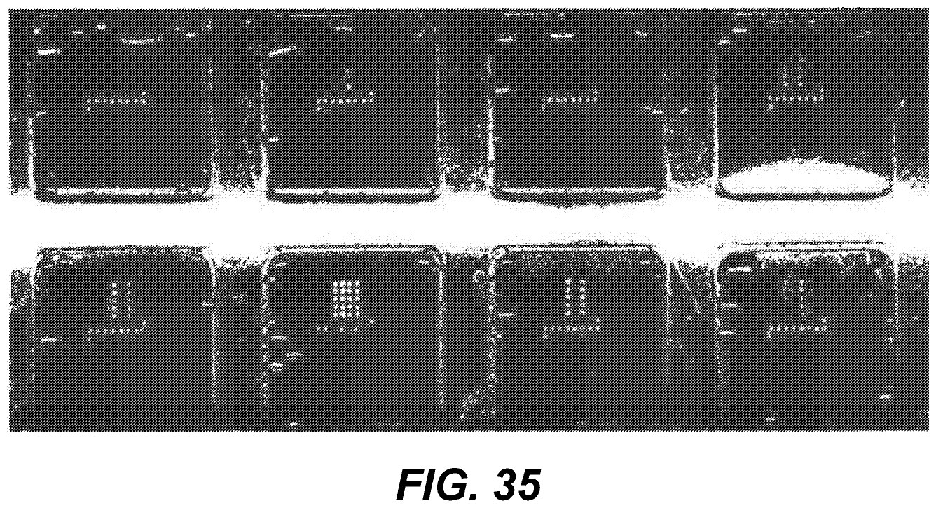

A representative scattering image that was used for the data analysis is shown in FIG. 35. Data for both 15 μM and 50 μM spots were analyzed, and all data plotted in this summary are averaged over five spots for each printed reagent.

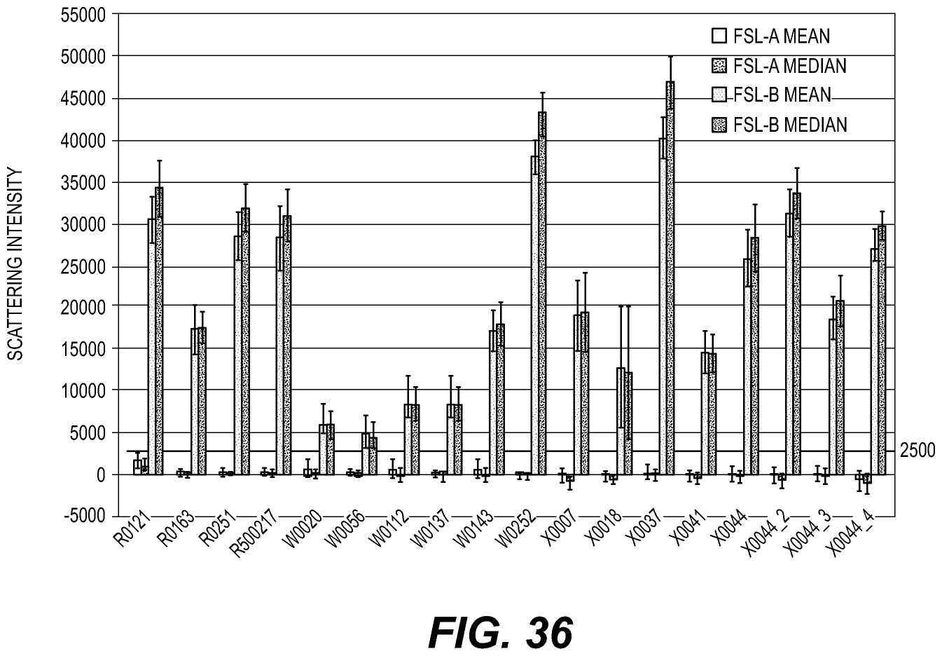

50 pM: The mean and median background-subtracted scattering intensity of 50 pM FSL-antigens exposed to Type A donors are shown In FIG. 36. The mean and median values were very similar, with the median values being slightly higher at higher intensities and slightly lower at lower intensities. The mean scattering Intensity of the expected positive spot, FSL-B, was greater than or equal to 5000 for all of the donors. The signal for the expected negative spot was less than 2500 for all donors, and less than 1000 for 17 out of 18 samples. Because the mean and median values were similar, only the mean values will be shown in the remaining graphs. However, all of the median scattering intensity data is available in the shared drive.

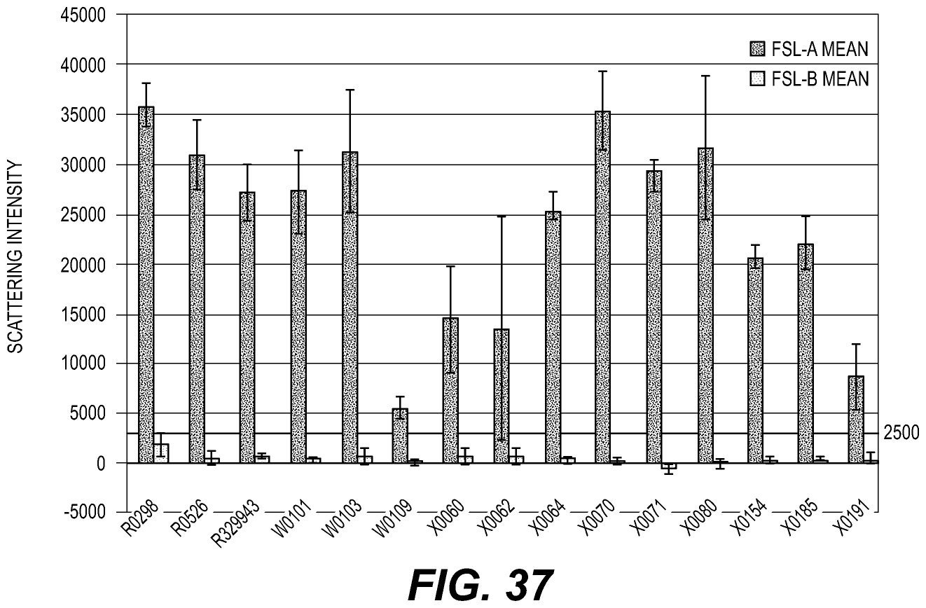

The mean background-subtracted scattering intensity of 50 μM FSL-antigens exposed to Type B donors is shown in FIG. 37. The mean scattering intensity of the expected positive spot, FSL-A, was greater than 5000 for all of the donors. The signal for the expected negative spot was less than 2500 for all donors, and less than 1000 for 14 out of 15 samples.

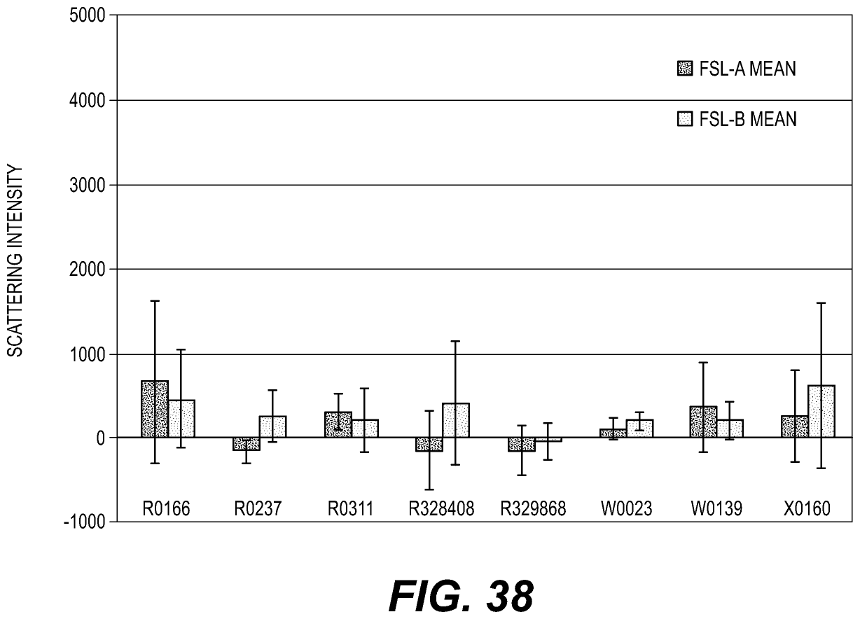

The mean background-subtracted scattering intensity of 50 μM FSL-antigens exposed to Type AB donors is shown in FIG. 38. Both FSL-A and FSL-B spots exhibited signals less than 1000, which was consistent with the expectation that they would both be negative when exposed to Type AB plasma donors.

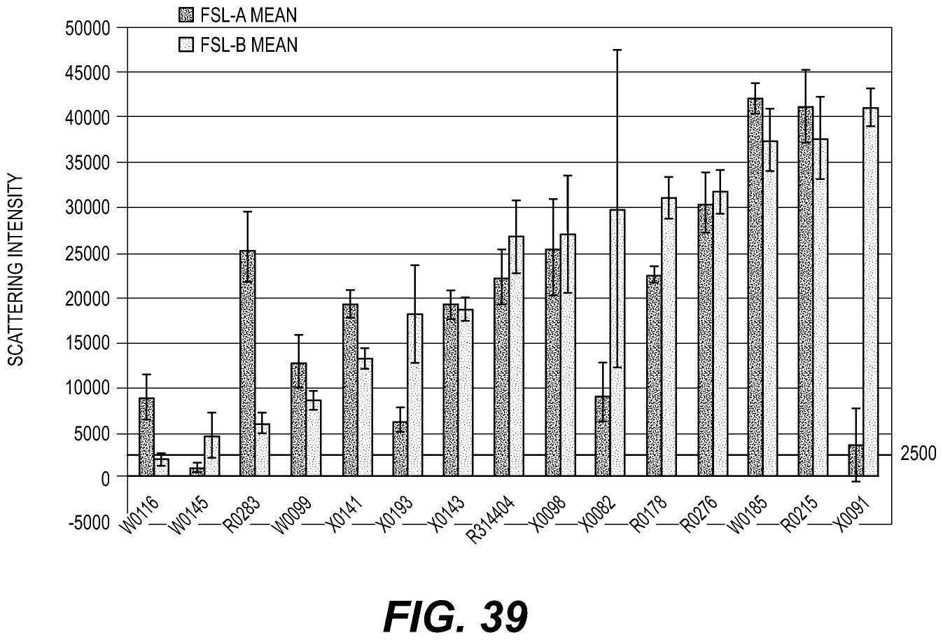

The mean background-subtracted intensity of 50 μM FSL-antigens exposed to Type O donors is shown in FIG. 39. All FSL-antigen spots exhibited signals greater than 1000, which was consistent with the expectation that both FSL-A and FSL-B spots would be positive when exposed to Type O plasma. A few of the spots had very sparse binding, however, resulting in signals near or below 2500.

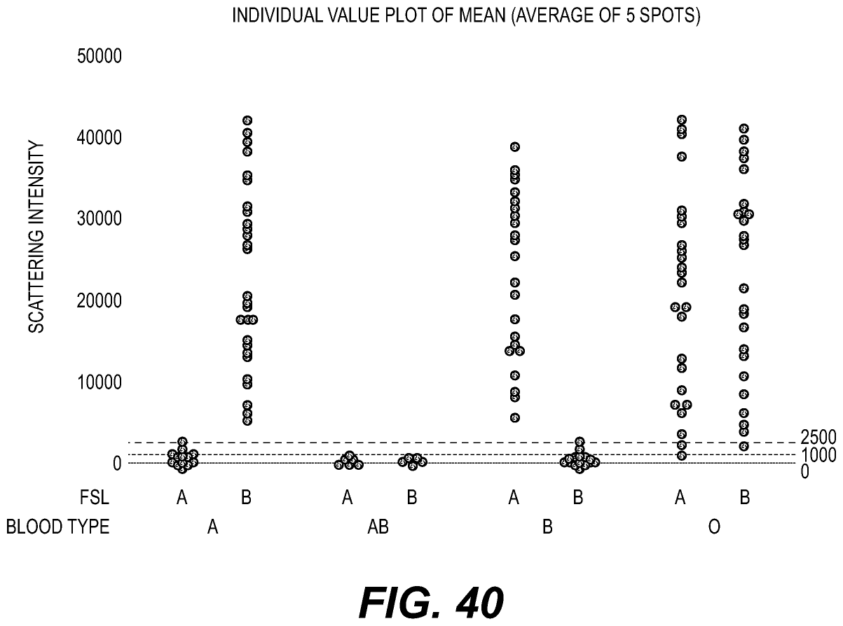

An overview of the data for both 15 μM and 50 μM FSL-antigen print concentrations, grouped by blood type, is shown in FIG. 40, with reference lines at 0 (black), 1000 (red), and 2500 (green). In general, there was good separation between the negative spots and the positive spots. As mentioned previously, there were a few donors with negative spots above 1000 and two Type O donors with positive spots below 2500, and those donors could be mistyped depending on which cutoff value is selected.

### Reproducibility of the Assay for a Single Plasma Donor Across all Four Slides

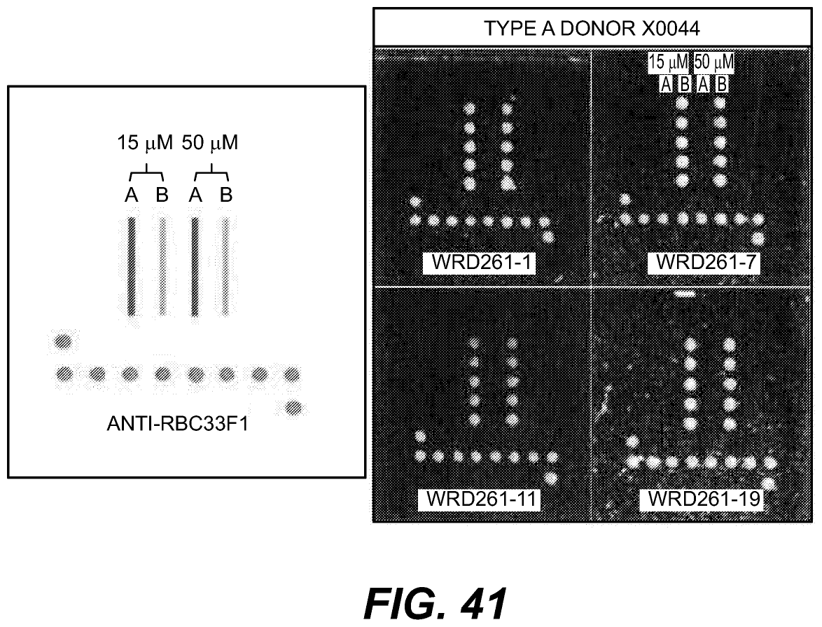

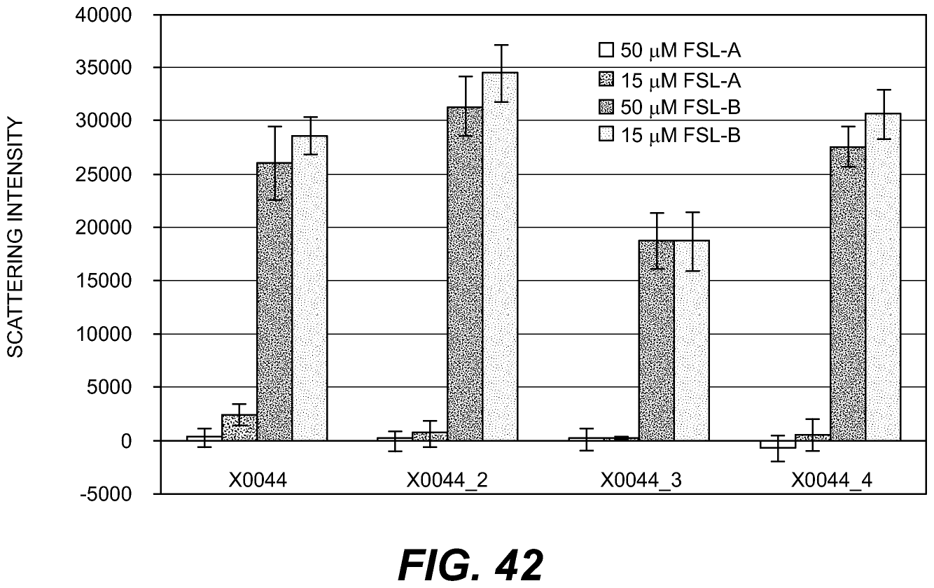

To determine the reproducibility of the assay, high titer Type A donor X0044 was run in well 1 of all four slides. Scattering images of wells exposed to donor X0044 are shown in FIG. 41 and the quantified data are shown in FIG. 42. The image captured by the camera is a combination of scattering from the detection cells bound to the printed spots, scattering from the solution in the well, and scattering from the tape and anything bound to the tape. Visually, there did not appear to be any non-specific binding to the printed FSL-A spots, but a streak down the column of the 15 μM FSL-A spots on slide WRD261-1 was observed. Since scattering from the spots cannot be isolated from scattering from the rest of the well using this imaging method, it was difficult to deduce the cause of the streak. There also appeared to be some differences in intensity in the spots, as well as the background, among the slides.

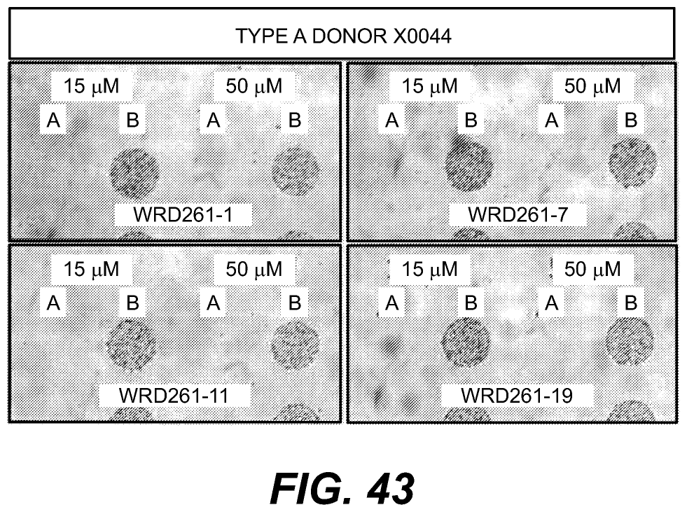

The quantified data for the 15 μM and the 50 μM FSL-antigen spots were similar. The scattering signal from the negative spots was very low with the exception of the 15 μM FSL-A column on slide WRD261-1, which was consistent with the streak observed in the scattering image. The intensity values for the FSL-B positive spots were similar from slide to slide with the exception of WRD261-11, which was approximately ⅔ of the intensity of the rest. Looking at the microscope images in FIG. 43, which are zoomed in on the first row of each array, the binding on the FSL-B spots does not appear to be significantly different between slides. These results suggest that the difference in signal intensity between slides may have been derived from the imaging method, but no definitive conclusion can be made. On slide WRD261-1, a smear is visible along the column where the 15 μM FSL-A spots were printed, which is consistent with both the scattering image and the quantified data from that image. It is unclear, however, if the smear was located on the surface of the slide or in another focal plane.

### Effect of FSL-Antigen Print Concentration

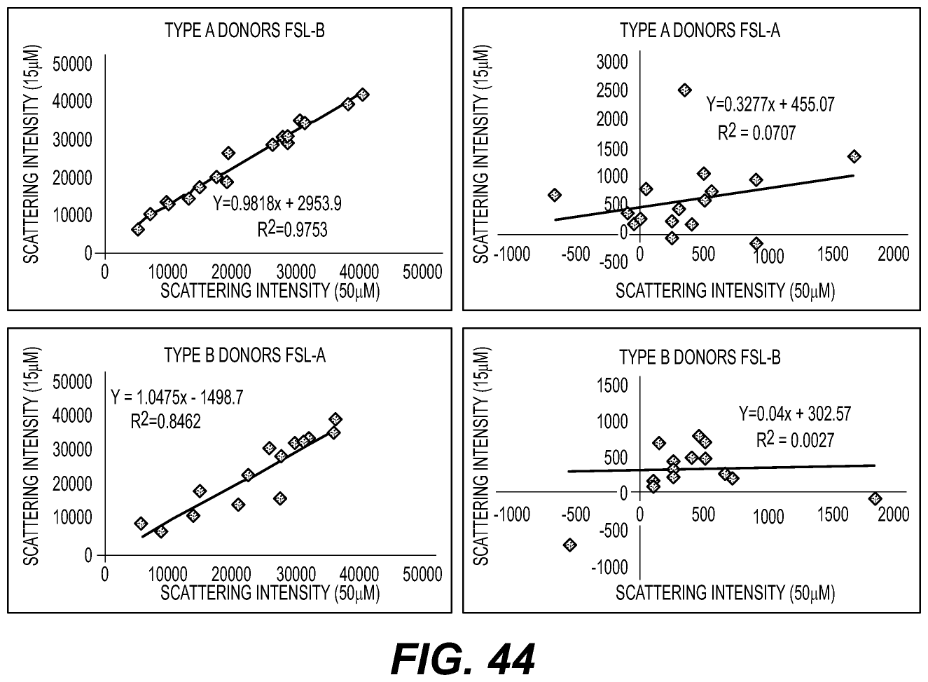

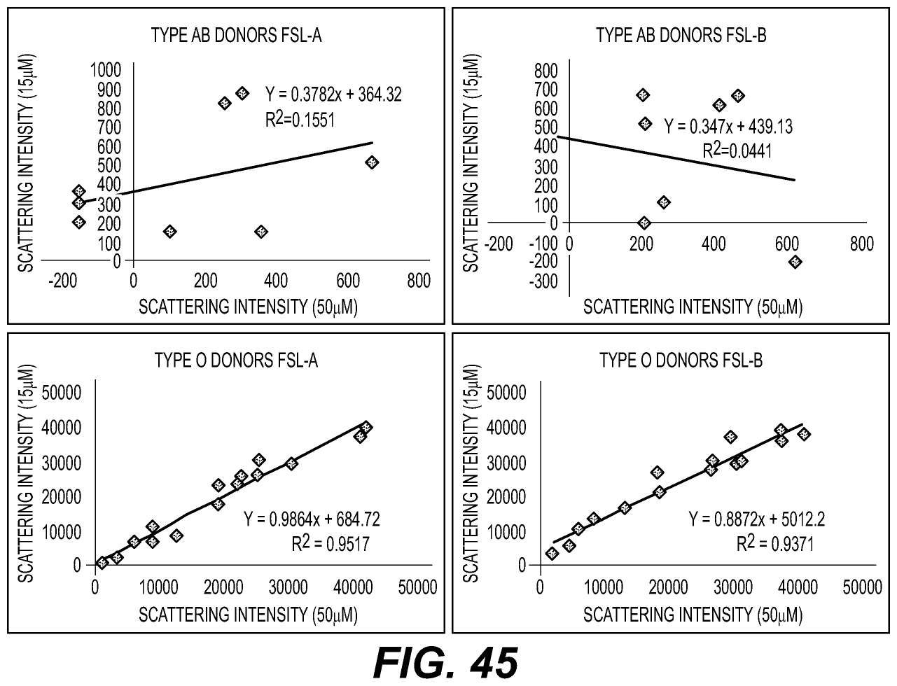

To examine whether there was any correlation between the scattering intensities exhibited by the 15 μM and 50 μM printed FSL-antigens, mean values were plotted against each other. These data are shown in FIG. 44 for Type A and Type B donors, and in FIG. 45 for Type AB and Type O donors. The graphs for all of the expected negative spots (FSL-A for Type A donors, FSL-B for Type B donors, and FSL-A and FSL-B for Type AB donors) indicate that there was no correlation between the FSL-antigens printed at 15 μM and 50 μM.

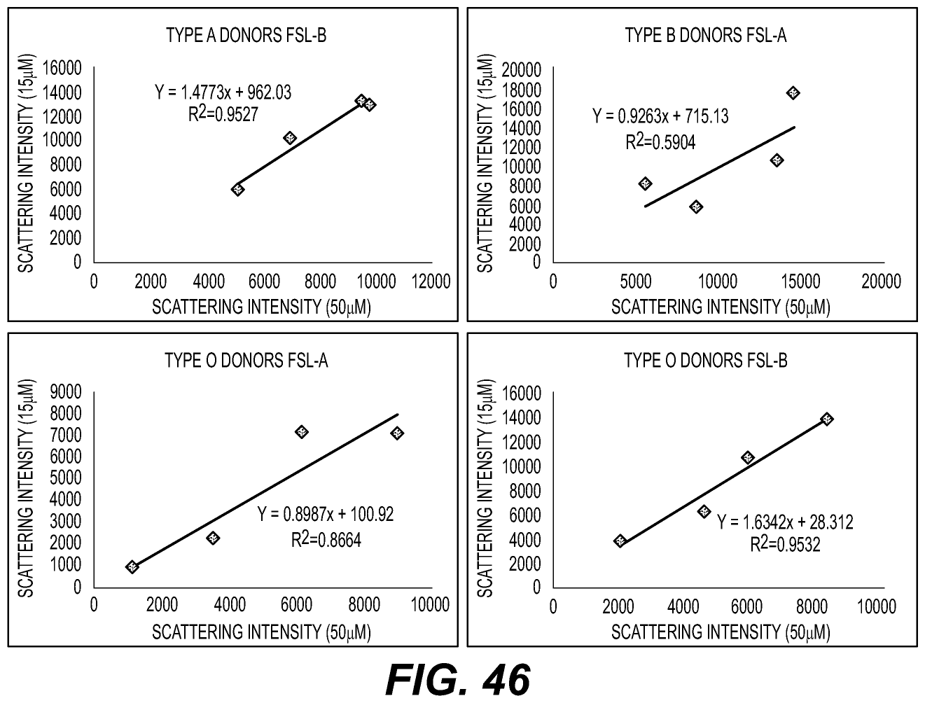

The graphs for the expected positive spots (FSL-B for Type A donors, FSL-A for Type B donors, and FSL-A and FSL-B for Type O donors) show that there is some correlation between the FSL-antigens printed at 15 μM and 50 μM, but they are difficult to interpret in terms of which print concentration gives the best results. Determining the best print concentration is most important for the donors that exhibit weak binding, and therefore lowest scattering intensity signal. Graphs for the donors resulting in the four lowest intensity spots were re-plotted in FIG. 46. There appears to be relatively good correlation between the concentrations for the FSL-B spots. The linear fits have slopes greater than 1, which suggests that for these lower intensity spots there is a different relationship between the two concentrations compared to the majority of samples with higher intensity spots. Examination of the data points indicates that the 15 μM spots resulted in a higher signal than the 50 μM spots for all 8 donors. In contrast, there appears to be poor or no correlation for the FSL-A spots. Examination of the data points suggests that the 50 μM spots resulted in a higher signal for some donors, while the 15 μM spots produced a higher signal for other donors.

Donors with Negative Spots Above 1000

Two A donors and one B donor were observed to have negative spots above 1000: X0044 (A) on slide WRD261-1, R0121 (A), and R0298 (B). Donor X0044 (A) was repeated on all four slides. The high scattering intensity value (2508.8) of the column of 15 μM FSL-A spots exposed to donor X0044 on slide WRD261-1 can be attributed to the smear that is visible in FIGS. 41 and 43. The fact that seven out of eight columns of negative spots exhibited intensities less than 1000 suggests that the smear was an artefact.

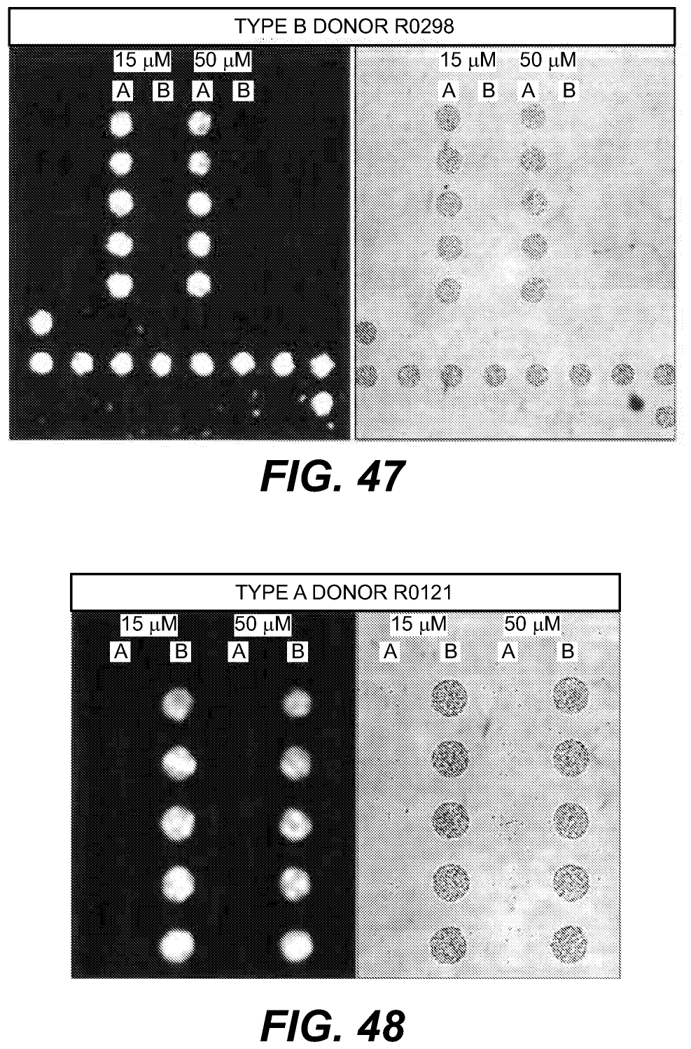

Donor R0298 (B) also had a value above 1000 for the negative spot, but for the 50 μM FSL-B spot only. The scattering and bright-field microscopy images for the array exposed to donor R0298 are shown in FIG. 47. No cells were bound to the FSL-B spots, suggesting that the high scattering may be a result of the imaging technique or the data analysis method.

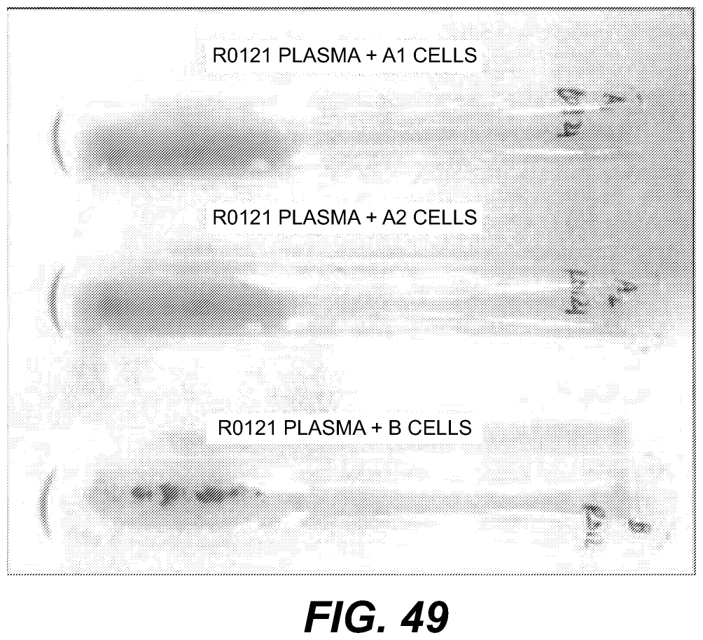

The third donor that exhibited a negative signal greater than 1000 was R0121, a Type A donor. In this case, both the 15 μM and 50 μM FSL-A spots exhibited high negative signal (1331.2 and 1689.6, respectively). A closer look at the scattering and bright-field microscopy images, shown in FIG. 48, reveal weak binding of cells localized to the printed FSL-A spots. Out of the 53 donors tested in this study, donor R0121 was the only one to exhibit any binding localized on the negative spots, as determined by bright-field microscopy. About 0.4% of Type A2 donors have anti-A1 antibodies (Giriyan, et al. J Clin Diagn Res 2017, 11(5), EC40-EC42), although donor R0121 plasma tested as an A in a reverse typing assay on the Echo, it is unknown whether it is subgroup A1 or A2. Tube testing showed no reaction of the plasma with A1 cells (FIG. 49), however, suggesting that R0121 does not contain anti-A1 and that the sparse binding observed was indeed non-specific binding or cross-reactivity.

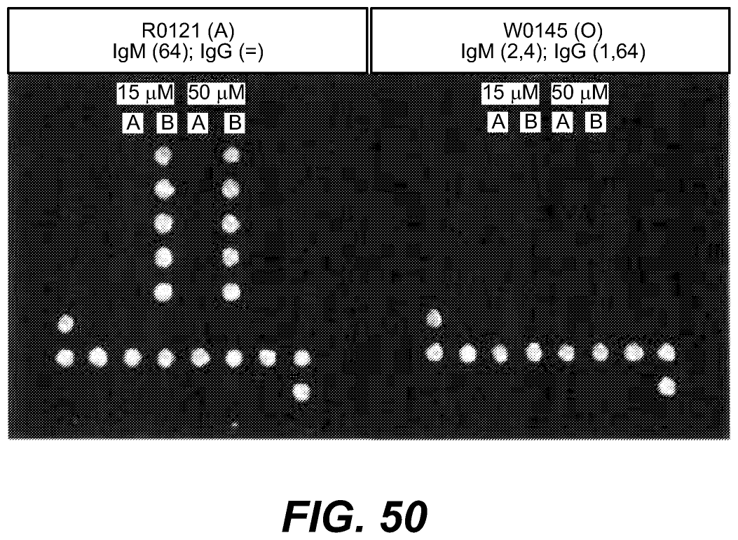

In general, the sparse binding observed on the negative FSL-A spots exposed to donor R0121 would not be an issue because the binding to positive spots exposed to most donors is stronger and can be distinguished from this weak non-specific binding. However, one of the Type O donors tested in this experiment exhibited very weak binding to the expected positive FSL-A spots. A comparison of the well exposed to R0121, where the FSL-A spot is expected to be negative, and the well exposed to W0145, where the FSL-A spot is expected to be positive, is shown in FIG. 50, and the two cannot be distinguished.

### Determining the Cutoff Value

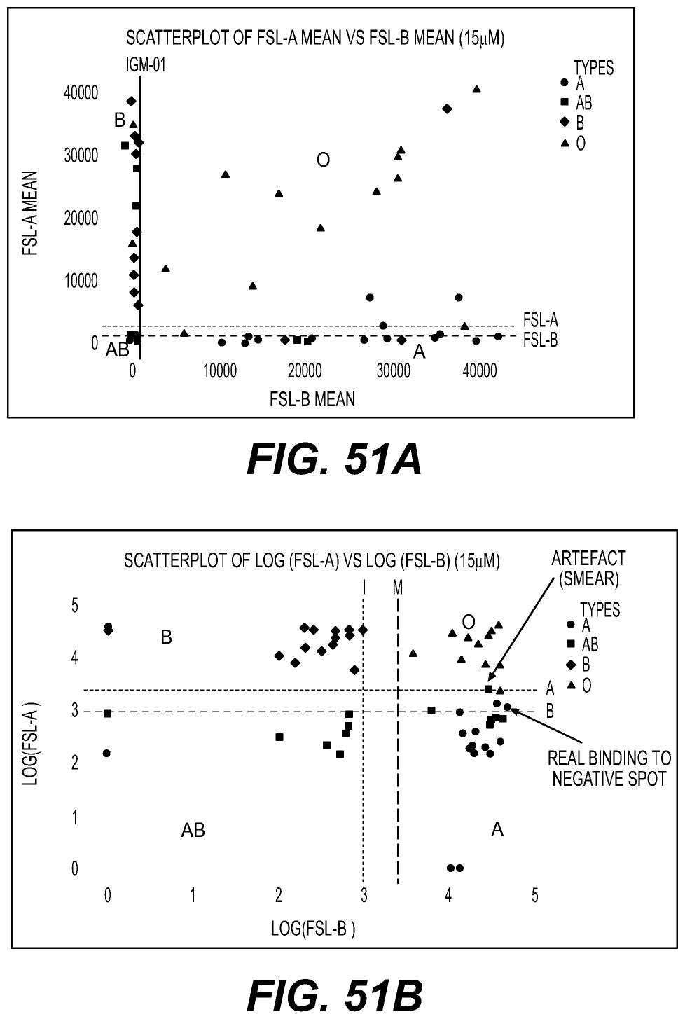

In order to select a cutoff value, the data were plotted as FSL-A vs FSL-B. The graph for the 15 μM FSL-antigen concentration is shown in FIG. 51A, with reference lines for potential cutoff values of 1000 (red line) and 2500 (blue line). The data form four distinct groups. The Type AB donors, which are expected to be negative for anti-A and anti-B, are located at the bottom left of the graph close to 0 (red squares). The Type A donors, which are expected to be negative for anti-A and positive for anti-B, are located at the bottom of the graph (blue circles). The Type B donors, which are expected to be positive for anti-A and negative for anti-B, are located on the left of the graph (green diamonds). The Type O donors, which are expected to be positive for both anti-A and anti-B, are scattered over the rest of the graph (purple triangles). For better visualization of the data points near the cutoff values, the data were also plotted as log(FSL-A) vs log(FSL-B), shown in FIG. 51B. Because many of the background-subtracted scattering intensity values for the negative spots were 5.0, those intensity values were replaced with a value of 1 in order to include those donors in the graph. In the log plot, the four distinct groups lie in four quadrants. If a cutoff value of 1000 was considered, all of the AB donors fall under this value. All of the B donors fall below the cutoff for the negative spot and above the cutoff for the positive spot. There is one Type O donor that has one positive spot below the cutoff, and two Type A donors which have a negative spot above the cutoff. In total, 3 out of 56 samples could not have been typed when using the 15 μM FSL-antigen scattering intensity values and a cutoff value of 1000. These samples have been discussed above: X0044, the repeated sample, had a smear on slide WRD261-1, R0121 exhibited legitimate non-specific binding on the negative FSL-A spots, and W0145 is a low titer O donor with very weak binding to FSL-A. Alternatively, if a cutoff value of 2500 is chosen, 2 out of 56 samples could not have been typed, both low titer Type O donors.

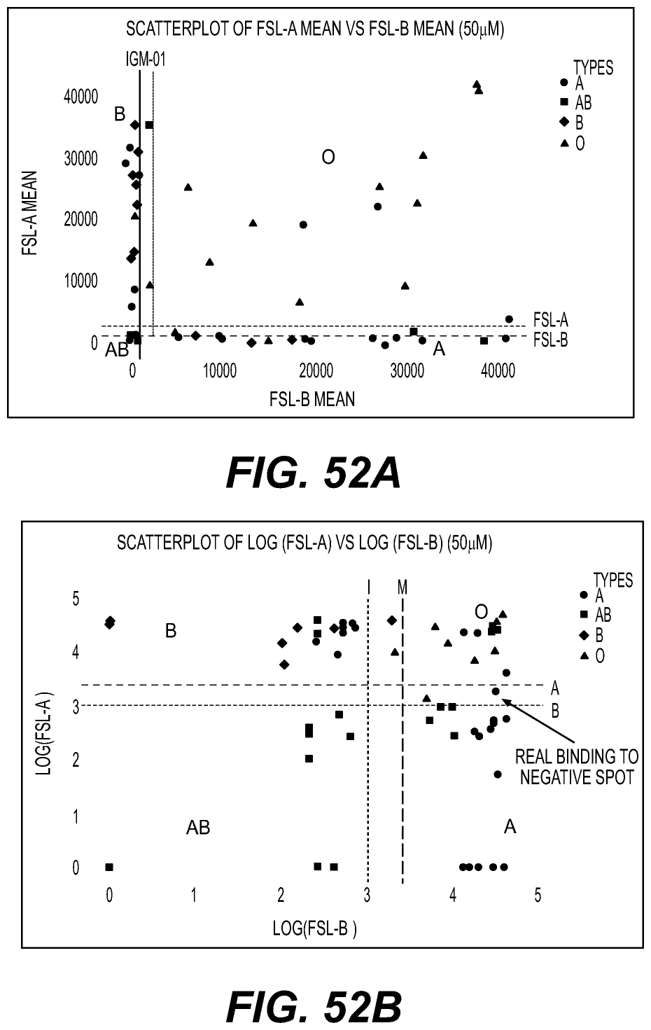

Similar plots for the 50 μM FSL-antigen spots showing similar results are shown in FIGS. 52A and 52B. Using a cutoff value of 1000, two out of 56 samples could not be typed, R0298 (B) and R0121 (A), both discussed above. If a cutoff value of 2500 is selected, those two donors are correctly typed, but two Type O donors, W0145 (O) and W0116 (O), would not be typed instead, each having one expected positive spot that falls below the cutoff value.

### Performance of the Assay

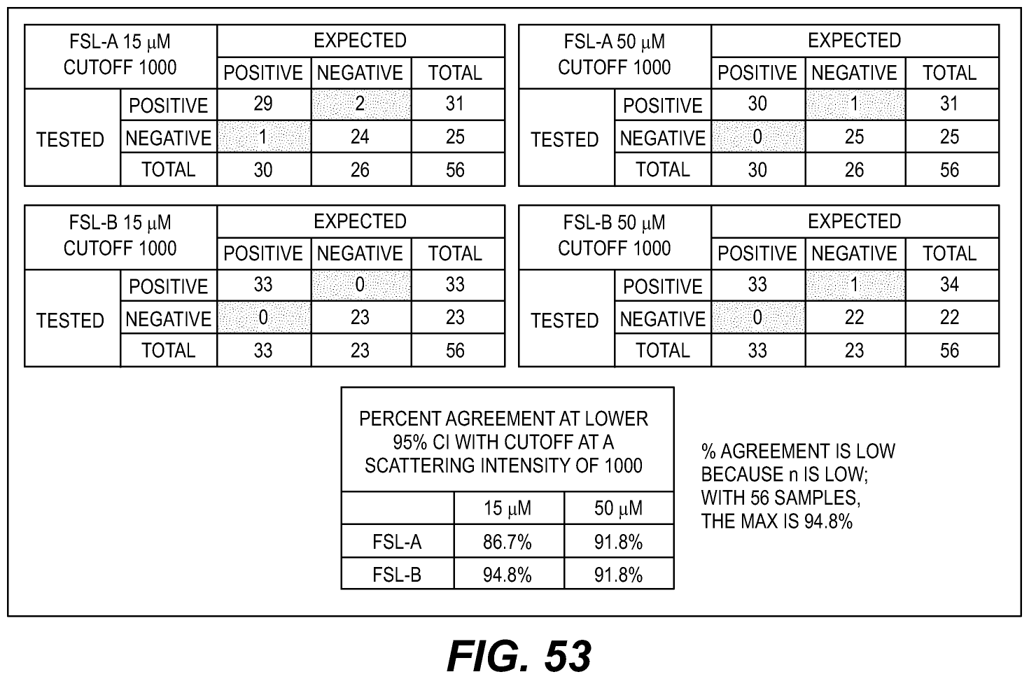

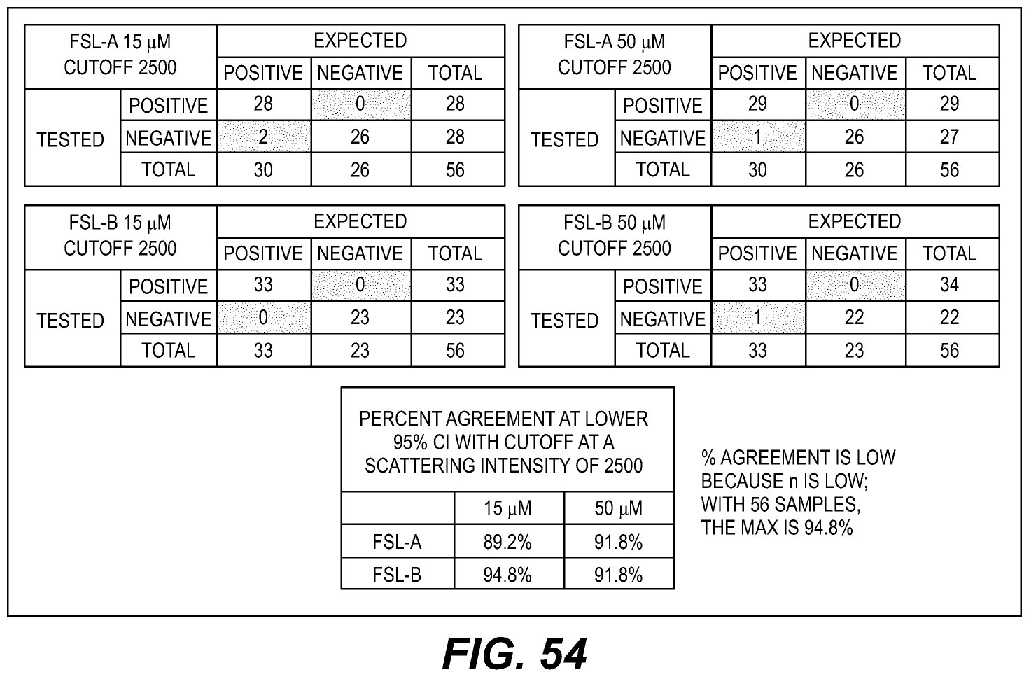

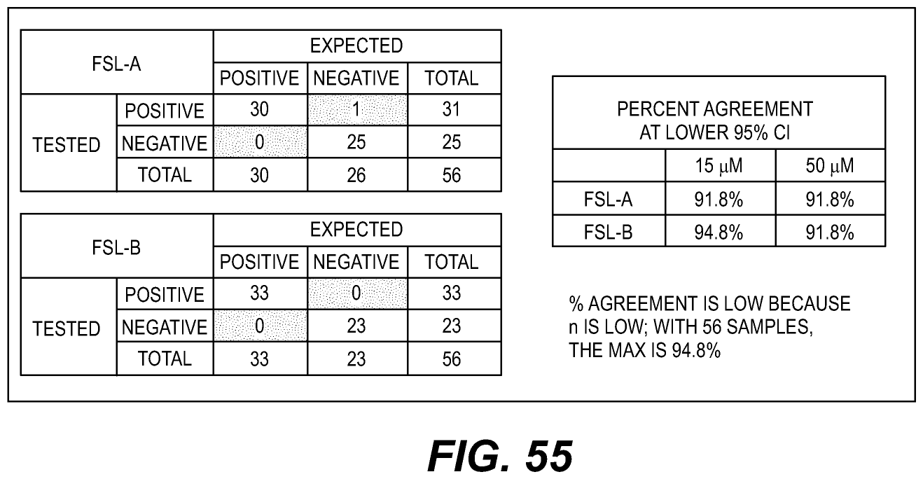

Contingency tables generated using a cutoff value of 1000 are shown in FIG. 53, and tables generated using a cutoff value of 2500 are shown in FIG. 54. From these contingency tables, a percent agreement was calculated at the lower bound of the 95% confidence interval. For a cutoff value of 1000, there was an 86.7% agreement for 15 μM FSL-A and a 94.8% agreement for FSL-B. For the 50 μM spots, there was a 91.8% agreement for both FSL-A and FSL-B. There was very little change in those numbers when a cutoff value of 2500 was selected (FIG. 54). When visual interpretation of the microscope images was used to generate the tables (FIG. 55), the numbers were a bit higher, with a 91.8% agreement for FSL-A and a 94.8% agreement for FSL-B, regardless of the print concentration. Although the numbers seem low, with n=56, the percent agreement at the lower bound of the 95% confidence interval cannot be higher than 94.8%.

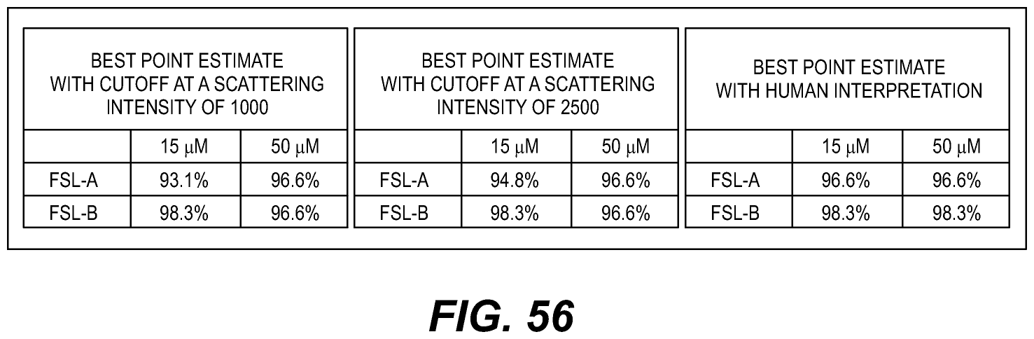

Because the number of samples was small, the best point estimate was also calculated for each scenario using the LaPlace method (https://measuring.com/wald/) and the tables are shown in FIG. 56.

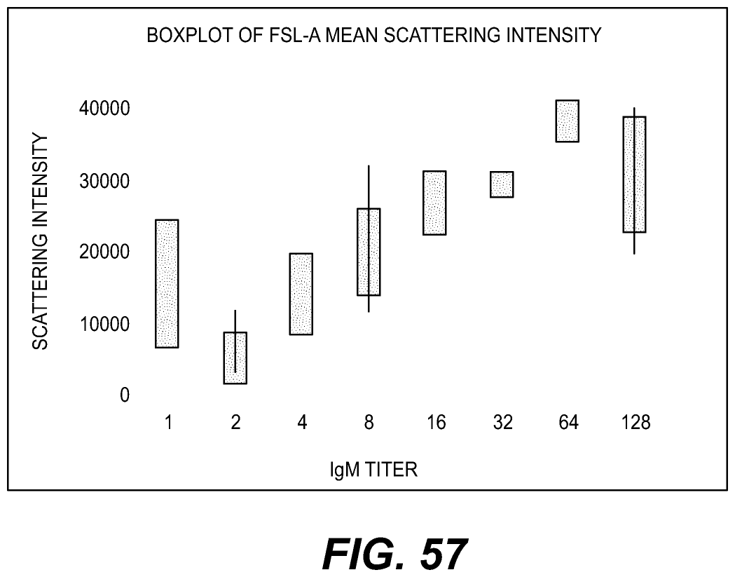

Correlation of scattering intensity with the anti-A or anti-B titer measured on the NEO: Theoretically, there should be a trend between the scattering intensity measured on the FSL-antigen spots and the titer of the plasma donors. The titration testing of anti-A and anti-B on the NEO (performed by F. Beyan) consists of two tests, one for IgM and one for IgG. The IgM test is an agglutination assay, and therefore likely measures some IgG in addition to the IgM. The signal exhibited by FSL-A spots was plotted vs the result of the IgM agglutination titration assay and the graph is shown in FIG. 57. Because of the limited number of samples per category of titer, all donors and FSL-antigen print concentrations with expected positive FSL-A spots were combined for the boxplot (all Type B and Type O donors). In general, there was an increase in intensity with increased anti-A titer.

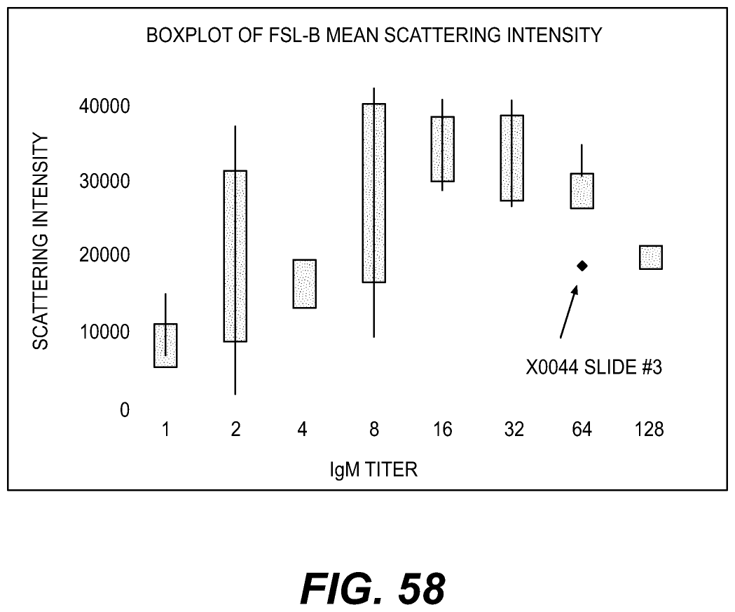

The signal exhibited by FSL-B spots for Type A and Type O donors was plotted vs the result of the IgM agglutination titration assay and is shown in FIG. 58. In general, there was an increase in intensity with increased anti-B titer but there was a slight drop at the high end. An outlier, donor X0044 run on slide WRD261-11, was identified by Minitab. As discussed in an earlier section on testing the reproducibility of the assay by running donor X0044 on all four slides, the scattering intensity observed on the array exposed to X0044 on the third slide was lower than the others.

### Conclusions

There was a 91.8% agreement for FSL-A and a 94.8% agreement for FSL-B at the lower 95% CI when interpreting the images visually (i.e., when excluding the wells that had a negative spot >1000, but no cells bound to the spot itself).

Using the quantified data from the scattering images, with no excluded data: For 50 μM spots, with a cutoff value of 1000 or 2500, there was a 91.8% agreement at the lower 95% CI for both FSL-A and FSL-B. For the 15 μM spots, there was an 86.7% agreement (cutoff of 1000) or 89.2% agreement (cutoff of 2500) for FSL-A and a 94.8% agreement for FSL-B.

### Example 13: Tp One-Step Antigen-Bridge Assay

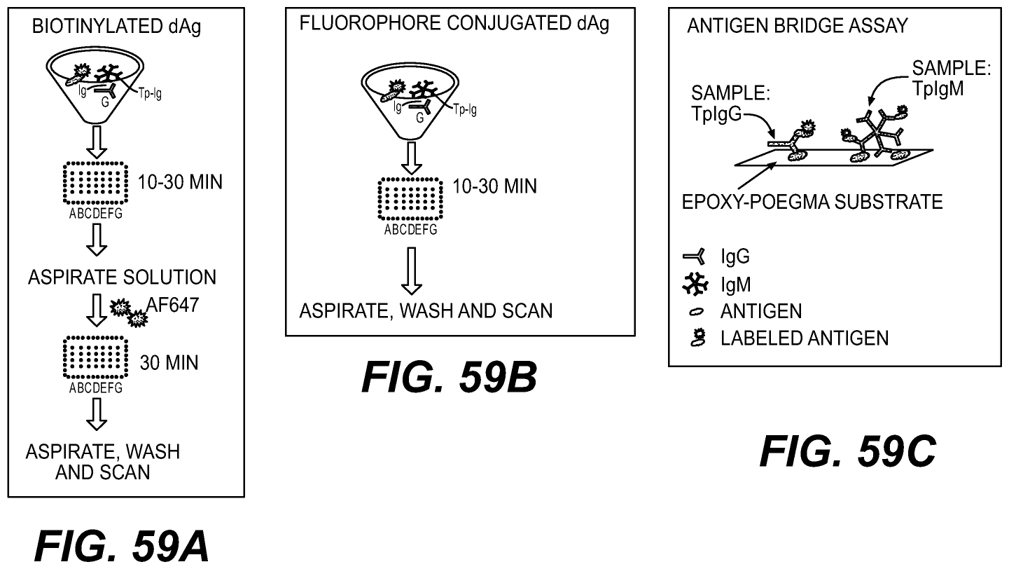

The Fluorophore conjugated dAg is a true one-step labeling with shorter TAT, less complex reagent preparation, and a simpler protocol. See FIG. 59A and FIG. 59B for a comparison. The principle of the antigen bridge assay is illustrated in FIG. 59C.

Purified protein (e.g., dAg) can be labeled with Alexa Fluor™ 647 Microscale Protein Labeling Kit (Thermo Fisher Scientific, Waltham, Mass.). An exemplary protocol is provided herewith. Briefly, desalt protein and reconstitute to 1 mg/mL solution. Calculate the appropriate volume of reactive dye stock solution to use. Prepare a 1 M sodium bicarbonate solution (pH 8.3). Transfer 20-100 μl (1 mg/ml) of protein (20-100 μg) to a reaction tube. Add 1/10 volume (2-10 μl) of 1 M sodium bicarbonate, and mix. Add 10 μl dH2O to one vial of Alexa Fluor 647 succinimidyl ester to reconstitute. Add the appropriate volume of reactive dye solution. Incubate the reaction mixture for 15 minutes at room temperature. Continue to conjugate purification using gel and spin column. Pack the gel resin in spin column. Add ≤50 μl of the conjugate reaction mixture onto the center of the resin. Centrifuge at 16,000×g for a total of 1 minute. Collection tube now contains purified dye-labeled protein. Proceed with determination of degree of labeling (DOL). In this example, two DOLs of AF647-R01549 conjugate were prepared: AF647-R01549 DOL8 and AF647-R01549 DOL12.

Next, proceed to the one-step direct conjugation of dAg with fluorophore. In brief, pre-wet array wells with 250 uL of incubation buffer (30 sec to 1 min). Aspirate at the end of pre-wetting. Add 250 uL sample (1:10) and dAgR01549-AF647 mixture (in incubation buffer) to well. Incubate on orbital shaker at RT for 30 minutes. Wash 1× in PBS-T 0.1% (wash buffer-1) using a Biotek-HBs01 (BioTek, Winooski, Vt.). Aspirate excess liquid manually. Remove the slide from the well assembly and move to 50 ml conical of PBS-T 0.1%; after 10 secs, move to second 50 ml conical of PBS-T 0.1%; after 10 secs, move to 50 ml conical of PBS (Wash buffer-2); after 10 secs, move to dry conical and centrifuge at 1000 g for 30 secs. Scan on the Genepix 4300A (Molecular Devices, San Jose, Calif.). Buffers used in the example include: (1) Incubation buffer: PBS-T (0.05% Tween-20) containing 1% BSA; (2) Dilution buffer: PBS containing 1% BSA; (3) Wash buffer-1: PBS-T (0.1% Tween-20); and (4) Wash buffer-2: PBS.

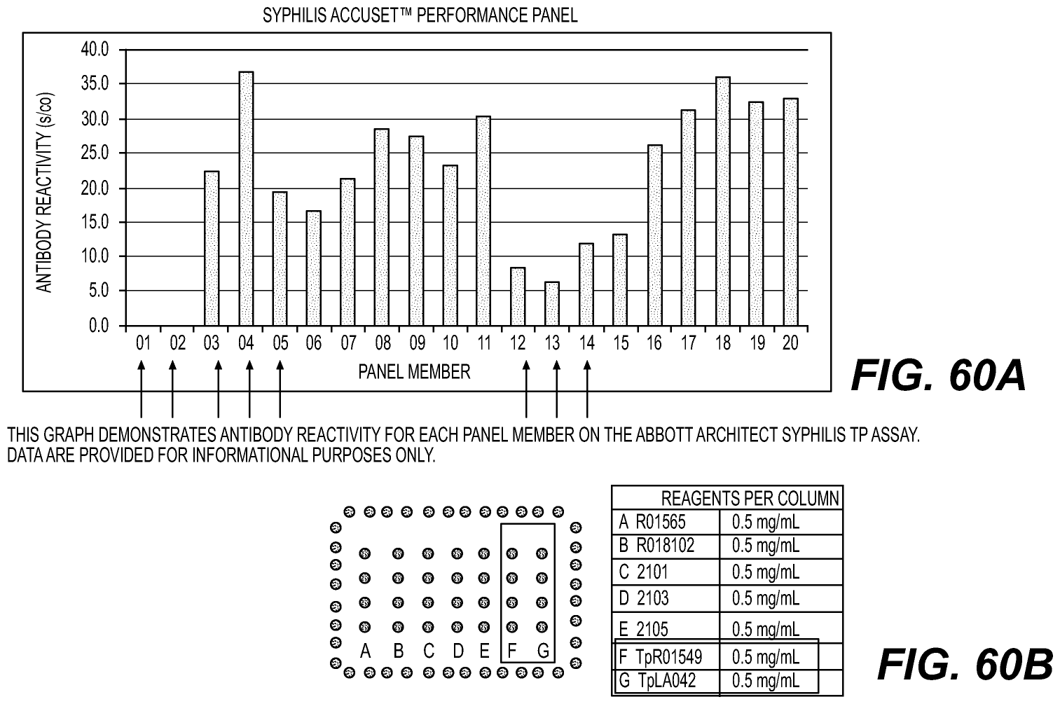

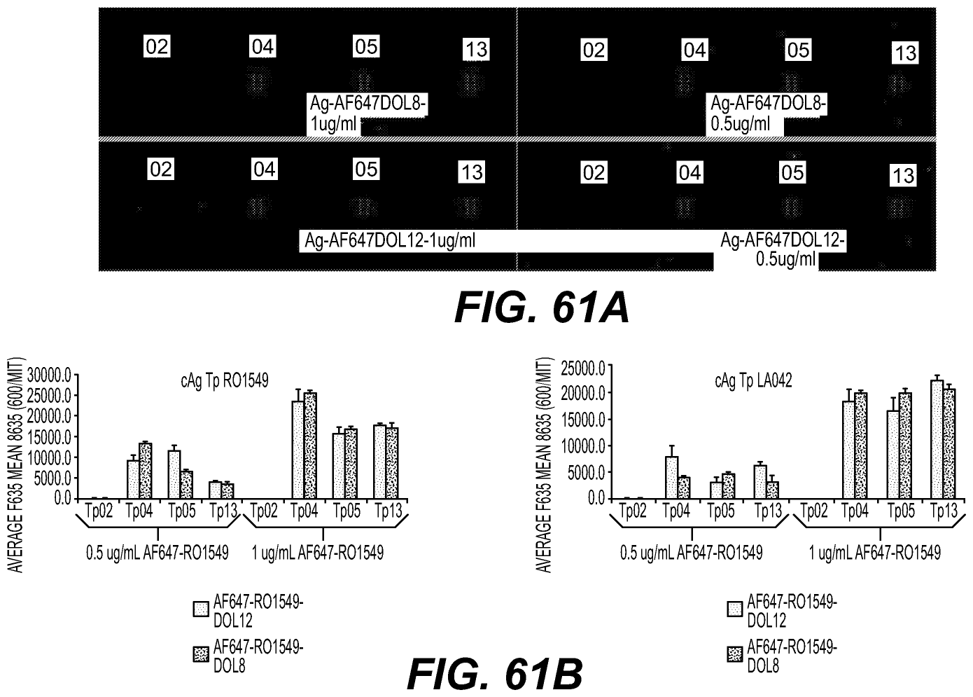

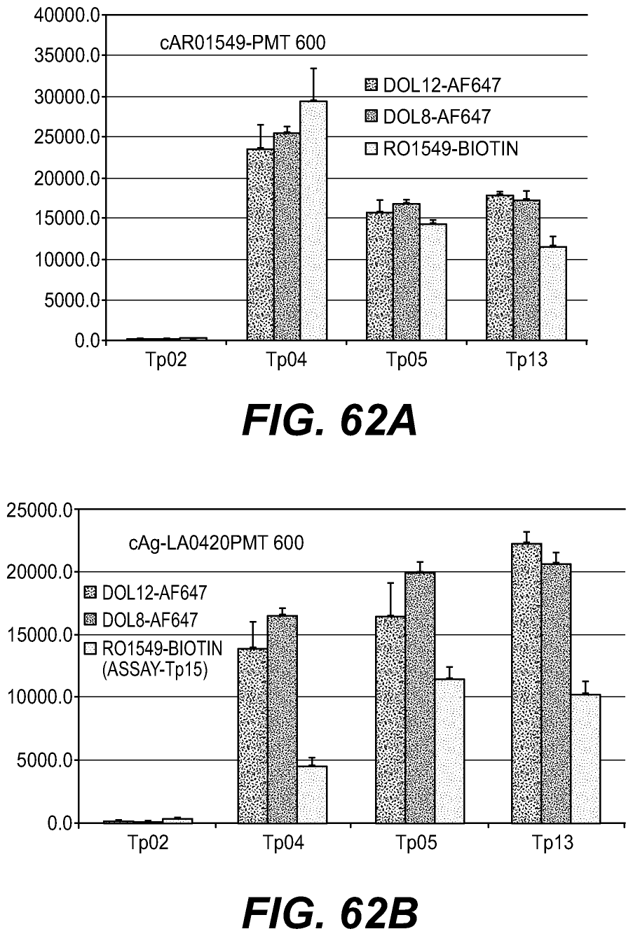

Samples used in this example were selected from SeraCare AccuSet™ Syphilis Performance panel (SeraCare Life Sciences, Gaithersburg, Md.) (FIG. 60A), sample numbers 01-05 and 12-14, marked by arrows. An array layout is illustrated in FIG. 60B. The Tp cAg R01549 and LA042 were in rows F and G. As Shown in FIG. 61A, all the positive samples were detected, i.e., sample numbers 04, 05, and 13. A quantitative illustration of the results is shown FIG. 61B. Detection with either 0.5 or 1 μg/ml of AF647-R01549 showed good differentiation between negative and positive samples. Further, a comparison of biotin labeled Ag (biotin-Ag, 0.5 μg/ml) versus AF647-Ag (1 μg/ml) is shown in FIG. 62A and FIG. 62B. The three bars from left to right are R01549-AF647DOL12, R01549-AF647DOL8, and R01549-Biotin, respectively.

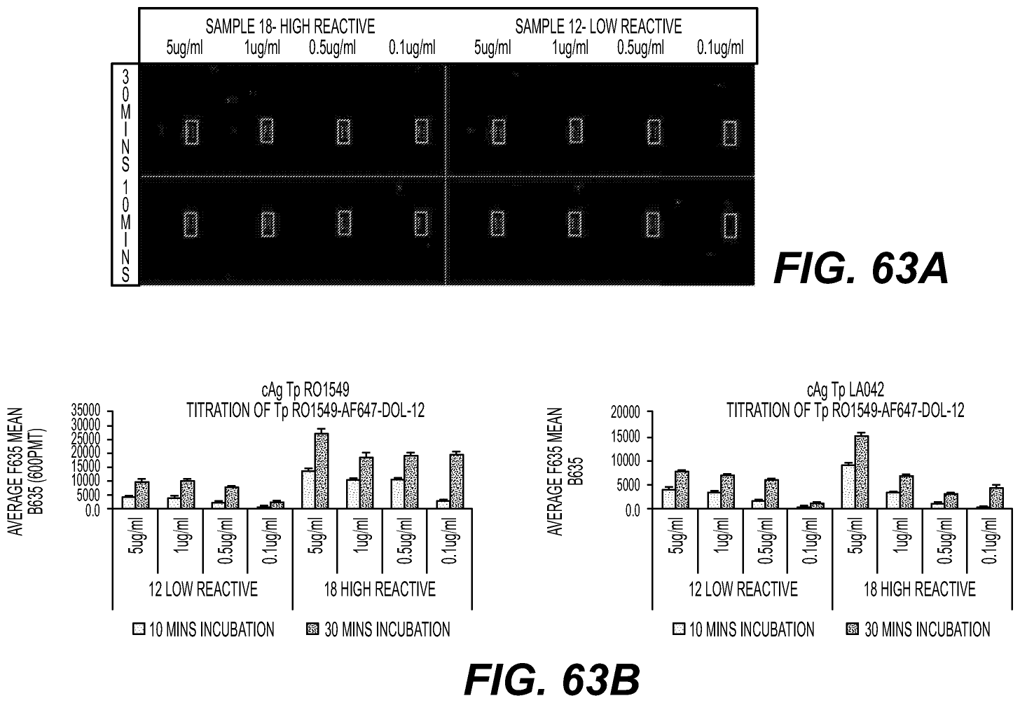

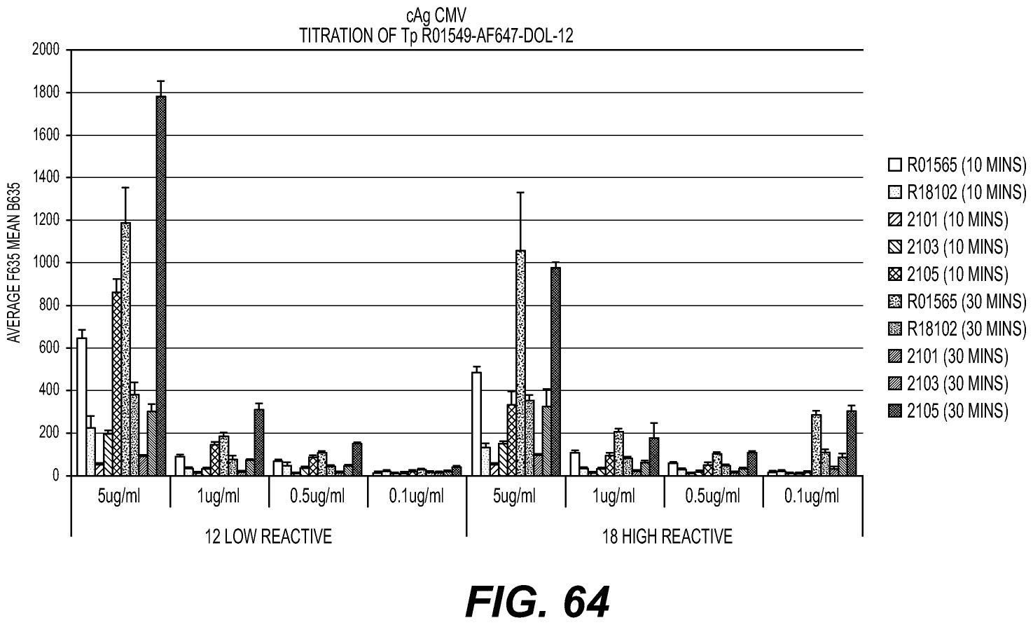

To test the effect of titration of direct-labeled Ag, low reactive sample 12 and high reactive sample 18 at concentrations of 5, 1, 0.5, and 0.1 μg/mg were used. The array was pre-wet. Samples (1:10) and AF647-Ag were added and held for 30 minutes. The array was then washed and read. Sample numbers 12 and 18 from the SeraCare Performance panel (FIG. 60A) and the same array layout (FIG. 60B) were used. The results and quantitative illustration are shown in FIG. 63A and FIG. 63B, respectively. In addition, an exemplary result for non-specific signals from CMV spots for each titration is shown in FIG. 64.

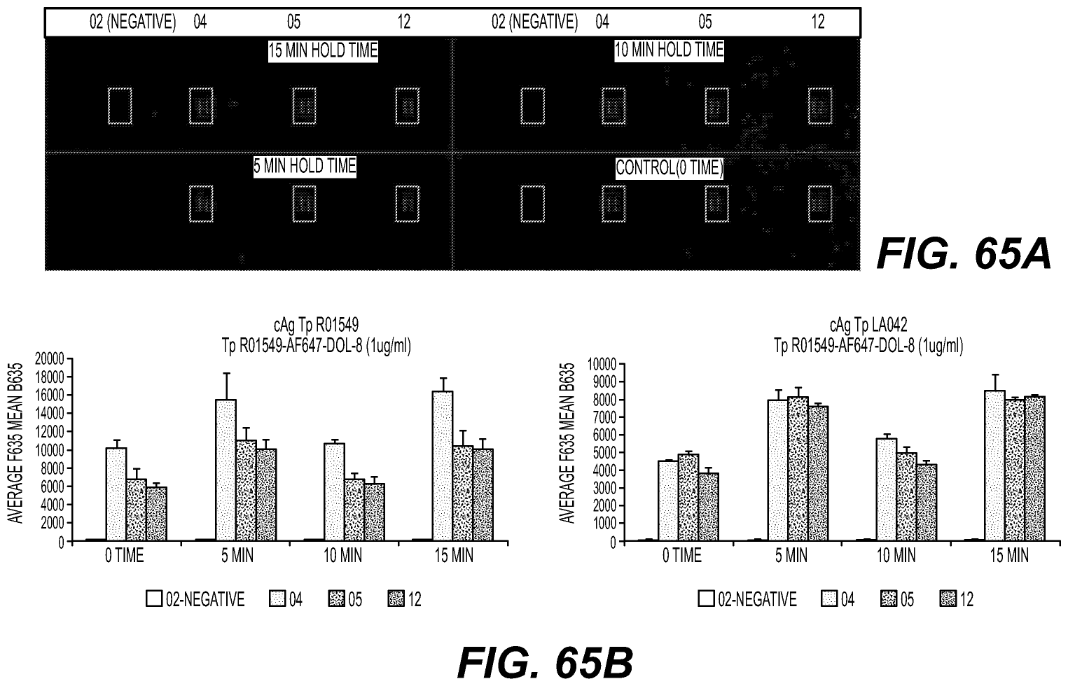

To test effect of hold time prior to array transfer, the same experiment setting was used. Holding time of 15, 10, 5, and 0 (control) minutes were tested. The results and quantitative illustration are shown in FIG. 65A and FIG. 65B, respectively.

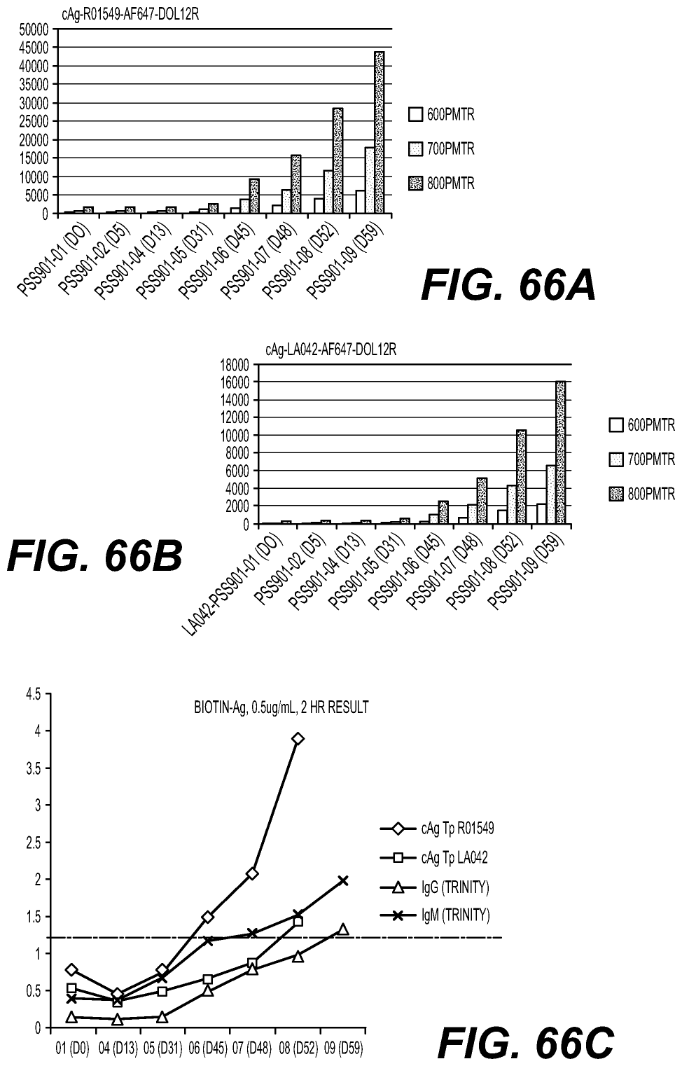

Further, a seroconversion panel test result is shown in FIGS. 66A-66C. Syphilis Seroconversion Panel PSS901 (AccuVert Syphilis Seroconversion Panel 0615-0017, SeraCare Life Sciences Inc., Milford, Mass.) is a 9-member panel of undiluted, naturally occurring plasma samples, collected over 58 days. This panel consists of serial bleeds from an individual plasma donor during the development of a Syphilis infection and subsequent response. Members in this panel convert negative to positive for RPR, Syphilis IgM, and Syphilis IgG.

In conclusion, Tp antigen R01549 direct conjugate with Alexa Fluor 647 used in Tp One-step reaction worked effectively. Signal intensity for cAg R01549 is comparable between AF647-R01549 and R01549-biotin. Signal intensity for cAg LA042 is lower with R01549-biotin as compared to AF647-R01549. In terms of titration, 1 μg/ml AF647-Ag generated good sensitivity and specificity on both low & high reactive sample. In contrast, 5 μg/ml generated more non-specific background on CMV cAg. Further, hold time of 5 or 15 minutes exhibited an increase in intensity (˜20-40%) compared to transfer without incubation time on both Tp R01549 and Tp LA042 cAgs. The 10-minute hold time showed a comparable intensity with no hold time on both cAgs. The data suggest that unintended dwell time has no negative impact in the assay. Surprisingly, it even gives stronger signal intensity.

The signal to noise increases seen on the ePOEGMA substrate surfaces are driven by the property of the polymer. That is to say that all assays will benefit from such surfaces. However, there are aspects of an assay that are driven by the reagents. For example the antibody may be cross reactive across several antigens, or may have very poor affinity etc. As such these limitations cannot be overcome by surface chemistry alone and requires alternative reagents/assay scheme. The use of dual antigen scheme is an example of such an alternative assay scheme that avoids the limitations of a traditional secondary antigen based antibody detection scheme. As the example illustrates, it is more specific (since the detected/captured antibody is sandwiched between two antigens that are specific for it) and faster (since the format allows for premixing of detection antigen and sample) than a traditional assay.

What is claimed is:

1. A composition comprising a biological sample and an ethylene glycol (EG) based polymer having an average molecular weight of less than about 2000 dalton when dissolved in the biological sample.

2. The composition of claim 1, wherein the EG based polymer has an average molecular weight of less than about 1000 dalton.

3. The composition of claim 1 or 2, wherein the EG based polymer has an average molecular weight of less than about 800 dalton.

4. The composition of any one of the preceding claims, wherein the EG based polymer has an average molecular weight of less than about 600 dalton.

5. The composition of any one of the preceding claims, wherein the EG based polymer has an average molecular weight average of less than about 400 dalton.

6. The composition of any one of the preceding claims, wherein the EG based polymer is selected from the group consisting of a polyethylene glycol (PEG), tetraethylene glycol, a triethylene glycol, a diethylene glycol, an ethylene glycol monomer, and a mixture of any of the forgoing.

7. The composition of any one of the preceding claims, wherein the EG based polymer has one or more end groups selected from the group consisting of dimethyl ether, diglycidyl ether (diepoxy), and methyl ether.

8. The composition of any one of the preceding claims, wherein the EG based polymer is selected from the group consisting of tetraethylene glycol dimethyl ether, PEG dimethyl ether, PEG diglycidyl ether (diepoxy), PEG methyl ether, and a mixture of any of the forgoing.

9. The composition of any one of the preceding claims, wherein the biological sample comprises blood, serum, plasma, lymph fluid, bile fluid, urine, saliva, mucus, sputum, tears, cerebrospinal fluid (CSF), bronchioalveolar lavage, nasopharyngeal lavage, rectal lavage, vaginal lavage, colonic lavage, nasal lavage, throat lavage, synovial fluid, semen, ascites fluid, pus, maternal milk, ear fluid, sweat, and amniotic fluid.

10. The composition of any one of the preceding claims further comprising one or more solvents.

11. The composition of claim 10, wherein the one or more solvent is water or PBS.

12. The composition of any one of the preceding claims, wherein the EG based polymer has a concentration in the range of about 0.5 mg/ml to about 20 mg/ml.

13. The composition of any one of the preceding claims, wherein the EG based polymer has a concentration in the range of about 1.0 mg/ml to about 10 mg/ml.

14. A non-fouling polymer layer comprising a brush polymer comprising a polymeric stem and a multitude of molecular bristles projecting from said polymeric stem, wherein the brush polymer comprises a co-polymer of an oligo ethylene glycol methacrylate (OEGMA) monomer and a methacrylate monomer (MAM) comprising a linking moiety and an electrophilic head group, wherein said co-polymer comprises a MAM to OEGMA v/v ratio from about 1:3 to about 1:8.

15. The non-fouling polymer layer of claim 14, wherein the MAM to OEGMA v/v ratio is about 1:4.

16. The non-fouling polymer layer of claim 14 or 15, wherein the OEGMA comprises poly(ethylene glycol) methacrylate (PEGMA) and poly(ethylene glycol) methyl ether methacrylate (PEGMEM).

17. The non-fouling polymer layer of claim 14, wherein said electrophilic head group is an epoxide group or an epoxy-ketone group.

18. The non-fouling polymer layer of any one of claims 14 to 17, wherein the MAM is glycidyl methacrylate (GMA).

19. The non-fouling polymer layer of any one of claims 14 to 18, wherein the co-polymer is epoxy-co-POEGMA.

20. The non-fouling polymer layer of any one of claims 14 to 19, wherein the co-polymer comprises GMA and PEGMEM, and wherein the GMA to PEGMEM ratio is about 1:4.

21. A device comprising
(a) a substrate comprising a surface;
(b) the non-fouling polymer layer of any one of claims 14-20 on the surface; and
(c) one or more capture regions on the non-fouling polymer layer, comprising at least one capture agent.

22. The device of claim 21, comprising a plurality of capture regions, wherein each capture region comprises at least one capture agent.

23. The device of claim 22, wherein the plurality of capture regions comprise at least two, three, or four different capture agents.

24. The device of claim 22 or 23, wherein each of the plurality of capture regions comprises a different capture agent.

25. The device of any one of claims 21-24, wherein the capture agent comprises a cell, a small molecule ligand, a lipid, a carbohydrate, a polynucleotide, a peptide, a protein, an antigen, or an antibody.

26. The device of claim 25, wherein the origin of capture agent is human, humanized, murine, chimeric, or synthetic.

27. The device of any one of claims 21-26, wherein the substrate is glass, silicon, a metal oxide, or a polymer.

28. The device of any one of claims 21-27, wherein the device comprises one or more compartments.

29. The device of claim 28, wherein the device comprises a plurality of compartments.

30. A detector comprising:
a body configured to accept the device of any one of claims 21 to 29;
a lid which, in combination with the body, substantially surrounds the chip when the device is disposed in the body;
a light source that is positioned to emit a light of a first wavelength such that the light contacts the non-fouling polymer layer;
a filter that is positioned to filter light of a second wavelength emitted from the non-fouling polymer layer;
a lens that is positioned to magnify a light of the second wavelength that passes through the filter; and
a power source that provides power for the light source.

31. The detector of claim 30, wherein the detector is a microarray detector or a nanoarray detector.

32. The detector of claim 30 or 31, wherein the detector has a volume of approximately 20-30 cm³.

33. The detector of claim 32, wherein the detector has a volume of about 25 cm³.

34. The detector of any one of claims 30 to 33, wherein the detector is self-contained.

35. The detector of any one of claims 30 to 34, wherein said detector is disposable.

36. A method of manufacturing a device, comprising:
(a) providing a substrate comprising a surface; and
(b) forming on the surface the non-fouling polymer layer of any one of claims 14-20.

37. The method of claim 36, further comprising printing at least one capture agent onto the non-fouling polymer layer.

38. The method of claim 36, further comprising printing a plurality of capture agents onto the non-fouling polymer layer.

39. The method of any one of claims 36 to 38, wherein the substrate is glass, silicon, a metal oxide, or a polymer.

40. A method for analyzing a biological sample comprising:
(a) contacting the biological sample with an ethylene glycol (EG) based polymer having an average molecular weight of less than about 2000 dalton when dissolved in the biological sample, and
(b) contacting the biological sample with a non-fouling polymer layer.

41. The method of claim 40, wherein the EG based polymer has an average molecular weight of less than about 1000 dalton.

42. The method of claim 40 or 41, wherein the EG based polymer has an average molecular weight of less than about 800 dalton.

43. The method of any one of claims 40 to 42, wherein the EG based polymer has an average molecular weight of less than about 600 dalton.

44. The method of any one of claims 40 to 43, wherein the EG based polymer has an average molecular weight average of less than about 400 dalton.

45. The method of any one of claims 40 to 44, wherein the EG based polymer is selected from the group consisting of a polyethylene glycol (PEG), tetraethylene glycol, a triethylene glycol, a diethylene glycol, an ethylene glycol monomer, and a mixture of any of the forgoing.

46. The method of any one of claims 40 to 45, wherein the EG based polymer has one or more end groups selected from the group consisting of dimethyl ether, diglycidyl ether (diepoxy), and methyl ether.

47. The method of any one of claims 40 to 46, wherein the EG based polymer is selected from the group consisting of tetraethylene glycol dimethyl ether, PEG dimethyl ether, PEG diglycidyl ether (diepoxy), PEG methyl ether, and a mixture of any of the forgoing.

48. The method of any one of claims 40 to 47, wherein the biological sample comprises blood, serum, plasma, lymph fluid, bile fluid, urine, saliva, mucus, sputum, tears, cerebrospinal fluid (CSF), bronchioalveolar lavage, nasopharyngeal lavage, rectal lavage, vaginal lavage, colonic lavage, nasal lavage, throat lavage, synovial fluid, semen, ascites fluid, pus, maternal milk, ear fluid, sweat, and amniotic fluid.

49. The method of any one of claims 40 to 48, further comprising one or more solvents.

50. The method of claim 49, wherein the one or more solvent is water or PBS.

51. The method of any one of claims 40 to 50, wherein the EG based polymer has a concentration in the range of about 0.5 mg/ml to about 20 mg/ml.

52. The method of any one of claims 40 to 51, wherein the EG based polymer has a concentration in the range of about 1.0 mg/ml to about 10 mg/ml.

53. The method of claim 40, wherein the non-fouling polymer layer comprises a brush polymer comprising a polymeric stem and a multitude of molecular bristles projecting from said polymeric stem, wherein the brush polymer comprises a co-polymer of an oligo ethylene glycol methacrylate (OEGMA) monomer and a methacrylate monomer (MAM) comprising a linking moiety and an electrophilic head group, wherein said co-polymer comprises a MAM to OEGMA v/v ratio from about 1:3 to about 1:8.

54. The method of claim 53, wherein the MAM to OEGMA v/v ratio is about 1:4.

55. The method of claim 53 or 54, wherein the OEGMA comprises poly(ethylene glycol) methacrylate (PEGMA) and poly(ethylene glycol) methyl ether methacrylate (PEGMEM).

56. The method of claim 53, wherein said electrophilic head group is an epoxide group or an epoxy-ketone group.

57. The method of any one of claims 53 to 56, wherein the MAM is glycidyl methacrylate (GMA).

58. The method of any one of claims 53 to 54, wherein the co-polymer is epoxy-co-POEGMA.

59. The method of any one of claims 53 to 58, wherein the co-polymer comprises GMA and PEGMEM, and wherein the GMA to PEGMEM ratio is about 1:4.

60. The method of any one of claims 53 to 59, wherein the non-fouling polymer layer further comprises one or more capture regions printed on the non-fouling polymer layer, comprising at least one capture agent.

61. The method of claim 60, wherein the non-fouling polymer layer comprises a plurality of capture regions, wherein each capture region comprises at least one capture agent.

62. The method of claim 61, wherein the plurality of capture regions comprise at least two, three, or four different capture agents.

63. The method of claim 61 or 62, wherein each of the plurality of capture regions comprises a different capture agent.

64. The method of any one of claims 60 to 63, wherein the capture agent is selected from a cell, a small molecule ligand, a lipid, a carbohydrate, a polynucleotide, a peptide, a protein, an antigen, an antibody, and a combination thereof.

65. The method of claim 64, wherein the origin of capture agent is human, humanized, murine, chimeric, or synthetic.

66. The method of claim 64, wherein the antigen is selected from at least one blood type antigen, at least one platelet antigen, at least one infectious disease antigen, at least one human leukocyte antigen (HLA), and any combination thereof.

67. The method of claim 66, wherein the at least one infectious disease antigen is selected from a human immune deficiency virus (HIV) antigen, a hepatitis B virus (HBV) antigen, a hepatitis C virus (HCV) antigen, a human T-lymphotropic virus (HTLV) antigen, a Treponema pallidum (TP) antigen, and any combination thereof.

68. The method of claim 66, wherein the at least one blood type antigen is selected from human A blood type antigen, a human B blood type antigen, a human AB blood type antigen, a human 0 blood type antigen, a human Rh factor antigen, a human MNS blood type antigen, a human P blood type antigen, a human P1PK blood type antigen, a human Lutheran blood type antigen, a human Kell blood type antigen, a human Lewis blood type antigen, a human Duffy blood type antigen, a human Kidd blood type antigen, a human Diego blood type antigen, a human Yt or Cartwright blood type antigen, a human Xg blood type antigen, a human Scianna blood type antigen, a human Dombrock blood type antigen, a human Colton blood type antigen, a human Landsteiner-Wiener blood type antigen, a human Chido/Rodgers blood type antigen, a human H blood type antigen, a human Hh/Bombay blood type antigen, a human Kx blood type antigen, a human Gerbich blood type antigen, a human Cromer blood type antigen, a human Knops blood type antigen, a human Indian blood type antigen, a human Ok blood type antigen, a human Raph blood type antigen, a human John Milton Hagen blood type antigen, a human I blood type antigen, a human li blood type antigen, a human Globoside blood type antigen, a human Gill blood type antigen, a human Rh-associated glycoprotein blood type antigen, a human Forssman blood type antigen, a human Langereis blood type antigen, a human Junior blood type antigen, and any combination thereof.

69. The method of claim 64, wherein the antibody is selected from IgG, IgM, IgA, IgD, IgE, and any combination thereof.

70. The method of claim 40, wherein contacting the biological sample with an ethylene glycol (EG) based polymer and contacting the biological sample with a non-fouling polymer layer occur sequentially or essentially simultaneously.

71. The method of claim 70, wherein contacting the biological sample with an ethylene glycol (EG) based polymer occurs prior to contacting the biological sample with a non-fouling polymer layer.

72. The method of claim 70, wherein contacting the biological sample with an ethylene glycol (EG) based polymer occurs post to contacting the biological sample with a non-fouling polymer layer.

73. The method of any one of claims 40-72, further comprising contacting the biological sample with one or more detection agents.

74. The method of claim 73, wherein the one or more detection agents comprise a first and a second detection agent.

75. The method of claim 73 or 74, wherein the one or more detection agents comprise one or more detection moieties selected from a chromophore, a fluorophore, a biotin, a radiolabel, a polynucleotide, a small molecule, an enzyme, a nanoparticle, a microparticle, a quantum dot, or an upconverter.

76. A kit comprising the composition of any one of claims 1-13, a set of buffers and/or reagents, and instructions for use.

77. A kit comprising the device of any one of claims 21-29, a set of buffers and/or reagents, and instructions for use.
